(19) United States
(12) Patent Application Publication
(10) Pub. No.: US 2013/0089554 A1
(43) Pub. Date: Apr. 11, 2013

(54) RON Binding Constructs and Methods of Use Thereof

(71) Applicants: John W. Blankenship, Seattle, WA (US); Philip Tan, Edmonds, WA (US); Sateesh Kumar Natarajan, Redmond, WA (US); Paul A. Algate, Issaquah, WA (US); Ruth A. Chenault, Mountlake Terrace, WA (US)

(72) Inventors: John W. Blankenship, Seattle, WA (US); Philip Tan, Edmonds, WA (US); Sateesh Kumar Natarajan, Redmond, WA (US); Paul A. Algate, Issaquah, WA (US); Ruth A. Chenault, Mountlake Terrace, WA (US)

(73) Assignee: Emergent Product Development Seattle, LLC, Seattle, WA (US)

(21) Appl. No.: 13/519,675

(22) PCT Filed: Dec. 29, 2010
(86) PCT No.: PCT/US10/62434

§ 371 Date: Dec. 7, 2012

Related U.S. Application Data
(60) Provisional application No. 61/290,840, filed on Dec. 29, 2009.
(60) Provisional application No. 61/365,266, filed on Jul. 16, 2010.
(60) Provisional application No. 61/366,743, filed on Jul. 22, 2010.

(51) Int. Cl. C07K 16/46

(52) U.S. Cl. CPC C07K 16/468
USPC 424/136.1; 530/387.3; 536/23.53; 435/320.1; 435/328; 435/254.21; 435/252.33; 435/252.31; 435/252.3; 435/252.35

(57) ABSTRACT

This disclosure provides immunoglobulin binding molecules that specifically bind to human macrophage stimulating receptor (MST1 R, also referred to herein as recepteur d'origine Nantaise or RON), including antibodies and monospecific and multispecific single chain binding proteins having one or more other domains, such as one or more antibody constant region domains. Also provided are therapeutic applications of such binding proteins, such as for the treatment of cancer and inflammatory disorders.

### CROSS-REFERENCE TO RELATED APPLICATION

This application claims the benefit under 35 U.S.C. §119(e) of U.S. Provisional Patent Application No. 61/290,840, filed Dec. 29, 2009, U.S. Provisional Patent Application No. 61/365,266 filed Jul. 16, 2010, and U.S. Provisional Patent Application No. 61/366,743, filed Jul. 22, 2010, each of which is incorporated by reference its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910180_{—}424PC_SEQUENCE_LISTING.txt. The text file is 746 KB, was created on Dec. 29, 2010, and is being submitted electronically via EFS-Web, concurrent with the filing of the specification.

### BAKGROUND

1. Technical Field

This disclosure relates generally to the field of binding molecules and therapeutic applications thereof and more specifically to a binding polypeptide comprising a binding domain that binds to RON (recepteur d'origine Nantaise), also referred to herein as macrophage stimulating 1 receptor or MST1R, and one or more other domains, such as one or more antibody constant region domains.

2. Description of the Related Art

RON (recepteur d'origine Nantaise, also known as MST1R) is a receptor-type protein tyrosine kinase that is essential to embryonic development and also plays an important role in inflammatory responses (Camp et al. Ann. Surg. Oncol. 12:273-281 (2005)). RON may play a role in controlling responses of macrophages during inflammation (Correll, P. H. et al., Genes Funct. 1997 February; 1(1):69-83). RON is mostly expressed in epithelial-derived cell types, and it has been suggested that RON, like a number of other receptor-type tyrosine kinases, may play a role in the progression of malignant epithelial cancers (Wang et al. Carcinogensis 23:1291-1297 (2003)).

Receptor-type protein tyrosine kinases generally consist of an extracellular domain which binds to extracellular ligands such as growth factors and hormones, as well an intracellular domain which possesses the kinase functional domain. Receptor-type protein tyrosine kinases have been sub-divided into a number of classes, and RON is a member of the MET family of receptor tyrosine kinases, which also includes Stk, c-Met and c-Sea (Camp et al. Ann. Surg. Oncol. 12:273-281 (2005)). RON and c-Met are the only members of the family found in humans, and they share about 65% homology overall. C-Met is the receptor for hepatocyte growth factor/scatter factor (HGF/SF) and has been fairly well characterized as a protooncogene.

RON is a transmembrane heterodimer comprised of one chain originating from a single-chain precursor and held together by several disulfide bonds. The intracellular part of RON contains the kinase domain and regulatory elements. The extracellular region is characterized by the presence of a semaphorin (sema) domain (a stretch of about 500 amino acids with several highly conserved cysteine residues), a PSI (plexin, semaphorins, integrins) domain, and four immunoglobulin-like folds.

The ligand for RON, macrophage stimulating protein (MSP) has also been identified and shares about 40% homology with the c-Met ligand, HGF/SF. MSP and HGF belong to the plasminogen-prothrombin family, which is characterized by kringle domains. MSP has also been linked with cancer. For example, Welm et al. observed an association between MSP and metastasis and poor prognosis in breast cancer (PNAS 104:7507-7575 (2007)).

RON and c-Met are the only receptor tyrosine kinases that have extracellular sema domains, and it has been demonstrated that the sema domain of RON includes its ligand binding site. Binding of MSP to RON causes phosphorylation within the kinase domain of RON, which leads to an increase in RON kinase activity. Alternatively, β₁ integrins can phosphorylate and activate RON through a Src-dependent pathway (Camp et al. Ann. Surg. Oncol. 12:273-281 (2005)). Activation of RON initiates signaling of a number of pathways, including PI3-K, Ras, src, β-catenin and Fak signaling. Many of the signaling pathways activated by RON are implicated in processes associated with cancer such as proliferation and inhibition of apoptosis.

RON itself has also been implicated in cancer progression for a number of reasons. For example, RON is expressed in a number of human tumors including breast, bladder, colon, ovarian and pancreatic cancers. In addition, RON has been shown in vitro to increase cell proliferation and motility. Furthermore, RON induces tumor growth and metastasis in RON-transgenic mice. (Waltz et al. Cancer Research 66:11967-11974 (2006)). Thus, there is a need for molecules that inhibit the RON signaling pathways.

### BRIEF SUMMARY

One aspect of the present disclosure provides an immunoglobulin binding polypeptide that specifically binds to human RON, wherein the immunoglobulin binding polypeptide comprises (a) a VL domain comprising i. a CDR1 amino acid sequence of SEQ ID NO:67, a CDR2 amino acid sequence of SEQ ID NO:68, and a CDR3 amino acid sequence of SEQ ID NO:69; or ii. a CDR1 amino acid sequence of SEQ ID NO:141, a CDR2 amino acid sequence of SEQ ID NO:142, and a CDR3 amino acid sequence of SEQ ID NO:143; or (b) a VH domain comprising i. a CDR1 amino acid sequence of SEQ ID NO:70, a CDR2 amino acid sequence of SEQ ID NO:71, and a CDR3 amino acid sequence of SEQ ID NO:72; or ii. a CDR1 amino acid sequence of SEQ ID NO:144, a CDR2 amino acid sequence of SEQ ID NO:145, and a CDR3 amino acid sequence of SEQ ID NO:146; or (c) a VL of (a) and a VH of (b). In one embodiment, the VL domain comprises an amino acid sequence of any one of SEQ ID NOS:80 or 152, and the VH domain comprises an amino acid sequence of any one of SEQ ID NOS:81, 153 and 176. In another embodiment, the VL and VH domains are humanized. In certain embodiments, the humanized VL comprises an amino acid sequence of any one of SEQ ID NOS:82, 83 and 154, and the humanized VH domain comprises an amino acid sequence of any one of SEQ ID NOS:84-86, 155 and 156.

In certain embodiments, the immunoglobulin binding polypeptide is an antibody or an antigen-binding fragment of an antibody. In this regard, the antibody or antigen-binding fragment of the antibody is non-human, chimeric, humanized or human.

In one embodiment of the immunoglobulin binding polypeptides of this disclosure, the non-human or chimeric antibody or antigen-binding fragment of the non-human or chimeric antibody has a VL domain comprising an amino acid sequence of any one of SEQ ID NO:80 and 152, and a VH domain comprising an amino acid sequence of any one of SEQ ID NO:81, 153 and 176.

In one embodiment of the immunoglobulin binding polypeptides of this disclosure, the humanized antibody or antigen-binding fragment of the humanized antibody has a VL domain comprising an amino acid sequence of any one of SEQ ID NOS:82, 83, and 154, and a VH domain comprising an amino acid sequence of any one of SEQ ID NOS:84-86, 155 and 156.

In another embodiment of the immunoglobulin binding polypeptides of the present disclosure, the antibody or antigen-binding fragment of the antibody comprises a VL domain that is at least about 90% identical to any one of the amino acid sequences of SEQ ID NOS:80, 82, 83, 152 and 154 and comprises a VH domain that is at least about 90% identical to any one of the amino acid sequences of SEQ ID NOS:81, 84-86, 153, 155, 156 and 176.

In certain embodiments of the immunoglobulin binding polypeptides of this disclosure, the binding polypeptide is selected from the group consisting of a Fab fragment, an F(ab′)2 fragment, an scFv, a dAb, and a Fv fragment. In certain embodiments, the scFv has a VL domain comprising an amino acid sequence of any one of SEQ ID NO:80 and 152, and has a VH domain comprising an amino acid sequence of any one of SEQ ID NO:81, 153 and 176. In another embodiment, the scFv is humanized and has a VL domain comprising an amino acid sequence of any one of SEQ ID NOS:82, 83 and 154, and has a VH domain comprising an amino acid sequence of any one of SEQ ID NOS:84-86, 155 and 156.

In one embodiment, of the immunoglobulin binding polypeptides of the present disclosure, the immunoglobulin binding polypeptide is a small modular immunopharmaceutical (SMIP) protein. In certain embodiments, the SMIP protein is non-human, chimeric, humanized or human. In certain embodiments, the non-human or chimeric SMIP protein has a VL domain comprising an amino acid sequence of any one of SEQ ID NO:80 and 152, and a VH domain comprising an amino acid sequence of any one of SEQ ID NO: 81, 153 and 176. In certain other embodiments, the humanized SMIP protein has a VL domain comprising an amino acid sequence of any one of SEQ ID NOS: 82, 83 and 154, and a VH domain comprising an amino acid sequence of any one of SEQ ID NOS:84-86, 155 and 156. In further embodiments, the immunoglobulin binding polypeptides of this disclosure comprise a hinge domain having an amino acid sequence of any one of SEQ ID NOS:349-366 and 420-475. In another embodiment, the immunoglobulin binding polypeptides of this disclosure comprise an immunoglobulin constant sub-region domain comprising an immunoglobulin CH2CH3 domain of IgG1, IgG2, IgG3, IgG4, IgA1, IgA2 or IgD. In a further embodiment, the immunoglobulin constant sub-region domain comprises human IgG1 CH2CH3. In one embodiment, the human IgG1 CH2 comprises the amino acid sequence of SEQ ID NO:241 and the human IgG1 CH3 comprises the amino acid sequence of SEQ ID NO:319.

In certain embodiments, the SMIP protein comprises a sequence that is at least 90% identical to the amino acid sequence of any one of the amino acid sequences selected from SEQ ID NOS:94-114 and 160-168.

In certain embodiments of the immunoglobulin binding polypeptides of the present disclosure, the immunoglobulin binding polypeptide is contained in a first single chain polypeptide comprising a first heterodimerization domain that is capable of associating with a second single chain polypeptide comprising a second heterodimerization domain that is not the same as the first heterodimerization domain, wherein the associated first and second single chain polypeptides form a polypeptide heterodimer. In certain embodiments, the polypeptide heterodimer comprises: a first single chain polypeptide comprising an amino acid sequence of SEQ ID NO:170, and a second single chain polypeptide comprising an amino acid sequence of SEQ ID NO:35; a first single chain polypeptide comprising an amino acid sequence of SEQ ID NO:172, and a second single chain polypeptide comprising an amino acid sequence of SEQ ID NO:27; a first single chain polypeptide comprising an amino acid sequence of SEQ ID NO:174, and a second single chain polypeptide comprising an amino acid sequence of SEQ ID NO:29; a first single chain polypeptide comprising an amino acid sequence of SEQ ID NO:174, and a second single chain polypeptide comprising an amino acid sequence of SEQ ID NO:32; a first single chain polypeptide comprising an amino acid sequence of SEQ ID NO:116, and a second single chain polypeptide comprising an amino acid sequence of SEQ ID NO:35; a first single chain polypeptide comprising an amino acid sequence of SEQ ID NO:118, and a second single chain polypeptide comprising an amino acid sequence of SEQ ID NO:27; a first single chain polypeptide comprising an amino acid sequence of SEQ ID NO:120, and a second single chain polypeptide comprising an amino acid sequence of SEQ ID NO:29; or a first single chain polypeptide comprising an amino acid sequence of SEQ ID NO:120, and a second single chain polypeptide comprising an amino acid sequence of SEQ ID NO:32.

In one embodiment of the immunoglobulin binding polypeptides of this disclosure, the immunoglobulin binding polypeptide is contained in a single-chain multi-specific binding protein comprising an immunoglobulin constant sub-region domain disposed between a first binding domain and a second binding domain, wherein the first binding domain is a human RON binding domain as described herein and the second binding domain is a human RON binding domain as described herein or is specific for a target molecule other than human RON. In certain embodiments, the immunoglobulin constant sub-region is IgG1 CH2CH3. In a further embodiment, the immunoglobulin constant sub-region is disposed between a first linker peptide and a second linker peptide. In a further embodiment, the first and second linker peptides are independently selected from the linkers provided in SEQ ID NOS:610-777. In yet a further embodiment, the first linker peptide comprises an immunoglobulin hinge region and the second linker peptide comprises a type II C-lectin stalk region.

In one embodiment, the immunoglobulin binding polypeptide of comprises the following structure: N-BD1-X-L2-BD2-C wherein: BD1 comprises an scFv specific for human RON; —X— is -L1-CH2CH3-, wherein L1 is an immunoglobulin IgG1 hinge having the amino acid sequence comprising any one of SEQ ID NOs:349-366, 420-475 and wherein —CH2CH3- is a human IgG1 CH2CH3 region or a variant thereof lacking one or more effector functions; L2 is a linker peptide having an amino acid sequence comprising any one of SEQ ID NOS:610-777; and BD2 is a binding domain specific for human RON or a target molecule other than human RON.

One aspect of the present disclosure provides a composition comprising one or more immunoglobulin binding polypeptides as described herein and a pharmaceutically acceptable excipient.

Another aspect of the present disclosure provides an expression vector capable of expressing the immunoglobulin binding polypeptides as described herein. A further aspect of the present disclosure provides a host cell comprising the expression vectors capable of expressing the immunoglobulin binding polypeptides as described herein.

Another aspect of the present disclosure provides a method for treating cancer comprising administering to a subject in need thereof a therapeutically effective amount of a composition comprising one or more immunoglobulin binding polypeptides as described herein and a pharmaceutically acceptable excipient. In this regard, the cancer is selected from the group consisting of pancreatic cancer, lung cancer, colon cancer and breast cancer, or other cancer as described herein.

Another aspect of this disclosure provides a method for treating an inflammatory disorder comprising administering to a subject in need thereof a therapeutically effective amount of a composition comprising one or more immunoglobulin binding polypeptides as described herein and a pharmaceutically acceptable excipient.

### BRIEF DESCRIPTION OF THE DRAWINGS

FIG. 1. RON-e01 and RON-f01 murine antibodies specifically bind human RON and cross-react with Macaca mulatta RON. NIH/3T3 cells transfected with empty vector (dashed), human RON (dotted) or Macaca mulatta RON (solid) were stained with secondary antibody alone (A), 1 mg/ml murine IgG (B), 1 mg/ml DX07 anti-RON antibody (C), RON-e01 anti-RON hybridoma supernatant (D) or RON-f01 anti-RON hybridoma supernatant (E).

FIG. 2. RON-e02 and RON-f02 murine SMIPs bind native Macaca mulatta RON on the surface of 4 MBr-5 cells. 4 MBr-5 cells were stained with secondary alone (dashed), the M0077 anti-CD79b SMIP (dotted), or anti-RON SMIP (solid).

FIG. 3. RON-e and RON-f murine SMIPs and Interceptors bind native human RON on the surface of BxPC-3 cells. BxPC-3 cells were stained with various concentrations of RON-e (A) or RON-f (B) molecules. See Tables 3 and 4 for description of SMIPS and Interceptors and associated SEQ ID NOs.

FIG. 4. RON-e01 and RON-f01 murine antibodies bind different epitopes within the extracellular domain of RON. RON-e01 antibody from hybridoma clone supernatants (1-5) does not bind recombinant RON Sema-PSI protein, indicating that part or all of the epitope recognized by RON-e01 lies outside of the Sema and PSI domains. Recombinant RON Sema-PSI protein binding is observed in all RON-f01 hybridoma clone supernatants (A-M) that contain measurable concentrations of IgG. “Diluent only” samples represent background binding in each assay when only serum diluent was run as the sample. As a positive control for IgG measurement and recombinant RON Sema-PSI binding, 250 ng/ml of an anti-human RON antibody (R&D Systems #MAB691, Minneapolis, Minn.) was tested in both ELISAs.

FIG. 5. RON-e and RON-f molecules bind RON at different epitopes. RON-e01: murine antibody; RON-f02: anti-RON SMIP; DX07: anti-RON n-chain antibody (Santa Cruz Biotechnology, Santa Cruz, Calif.).

FIG. 6A. RON-e01 antibody and RON-e05 YAE interceptor can inhibit MSP-induced phosphorylation of RON, Akt and MAPK.

FIG. 6B. RON-f01 antibody, RON-f02 SMIP and RON-f03 2^{nd} generation interceptor can inhibit MSP-induced phosphorylation of RON, Akt and MAPK.

FIG. 7. RON-e and RON-f humanized SMIPs bind native human RON on the surface of MDA-MB-453 cells. MDA-MB-453 cells were stained with various concentrations of RON-e (A) or RON-f (B) molecules. The humanized SMIPs have comparable binding activity as their murine counterparts.

FIG. 8A. RON-f humanized SMIPs can inhibit MSP-induced phosphorylation of RON, Akt and MAPK in MDA-MB-453 cells. RON-f humanized SMIPs cause minimal phosphorylation of RON but not of Akt or MAPK when applied during the blocking step (1 hour) and followed by mock stimulation.

FIG. 8B. Humanization of the RON-f02 murine SMIP reduces receptor phosphorylation in response to SMIP application during the stimulation step (20 min). RON-f02 murine SMIP stimulates RON phosphorylation but not downstream Akt or MAPK phosphorylation. The humanized SMIPs, RON-f07h24 and RON-f08h25, effect reduced RON phosphorylation compared to the murine SMIP. Interestingly, the high level of downstream effector protein phosphorylation observed in response to MSP-induced RON activation is not observed following SMIP-induced phosphorylation of the RON receptor.

FIG. 9: Bispecific proteins pairing a humanized RON-f binding domain with an anti-CD3 binding domain specifically direct cytotoxic T cell killing of target cells expressing the RON antigen. MDA-MB-453 (A) or Daudi (B) target cells were loaded with Chromium-51 and incubated with increasing concentrations of bispecific proteins in the presence of a 10:1 ratio of purified human T cells to target cells. Following a 4 hour incubation at 37° C., target cell lysis was assessed by the release of Chromium-51 into the assay supernatant. MDA-MB-453 cells, a human metastatic breast carcinoma line, express RON but not CD19 while Daudi cells, a human Burkitt's Lymphoma line, express CD19 but not RON. Both target cell lines are killed only when incubated together with T cells and a bispecific protein that binds an antigen expressed by the target cell. When the bispecific protein does not bind the target cell (i.e., an anti-RON×anti-CD3 bispecific with Daudi cells), no target cell cytotoxicity is observed. Data represent the mean of duplicates +/− standard error of the mean (SEM).

FIGS. 10A and 10B show binding of bispecific anti-RON and anti-CD3 constructs (polypeptide heterodimer S0268 and Scorpion protein S0266) to MDA-MB-453 cells (A) and to isolated T cells (B).

FIGS. 11A and 11B shows T-cell directed cytoxicity induced by bispecific polypeptide heterodimers TSC054, TSC078, TSC079, and S0268 in a chromium (⁵¹Cr) release assay with (A) Daudi (RON⁻, CD19⁺) cells or (B) BxPC-3 (RON⁺, CD19⁻) cells.

### DETAILED DESCRIPTION

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts the term's definition in the application, the definition that appears in this application controls.

This disclosure relates generally to the field of binding molecules and therapeutic applications thereof and more specifically to immunoglobulin binding polypeptides composed of a binding domain that binds to the macrophage stimulating 1 receptor (MST1R, also referred to herein as recepteur d'origine Nantaise or RON) and one or more other domains, such as one or more antibody constant region domains. As detailed further herein, the binding proteins may be any of a number of different formats, such as antibodies and antigen-binding fragments thereof, SMIP™, PIMS, Xceptor, SCORPION™, and Interceptor fusion protein formats.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” means±20% of the indicated range, value, or structure, unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. In addition, it should be understood that the individual fusion proteins derived from the various combinations of the components (e.g., domains) and substituents described herein, are disclosed by the present application to the same extent as if each fusion protein was set forth individually. Thus, selection of particular components of individual fusion proteins is within the scope of the present disclosure.

As used herein, a polypeptide or protein “consists essentially of” several domains (e.g., a binding domain that specifically binds a target, a hinge, a dimerization or heterodimerization domain, and an Fc region constant domain portion) if the other portions of the polypeptide or protein (e.g., amino acids at the amino- or carboxyl-terminus or between two domains), in combination, contribute to at most 20% (e.g., at most 15%, 10%, 8%, 6%, 5%, 4%, 3%, 2% or 1%) of the length of the polypeptide or protein and do not substantially affect (i.e., do not reduce the activity by more than 50%, such as more than 40%, 30%, 25%, 20%, 15%, 10%, or 5%) the activities of various domains (e.g., the target binding affinity of the binding domain, the activities of the Fc region portion, and the capability of the heterodimerization domain in facilitating heterodimerization). In certain embodiments, a polypeptide or protein (e.g., a fusion polypeptide or a single chain fusion polypeptide) consists essentially of a binding domain that specifically binds a target, a heterodimerization domain, a hinge, and an Fc region portion and may comprise junction amino acids at the amino- and/or carboxyl-terminus of the protein or between two different domains (e.g., between the binding domain and the heterodimerization domain, between the heterodimerization domain and the hinge, and/or between the hinge and the Fc region portion).

A “binding domain” or “binding region,” as used herein, refers to a protein, polypeptide, oligopeptide, or peptide that possesses the ability to specifically recognize and bind to a target (e.g., RON). A binding domain includes any naturally occurring, synthetic, semi-synthetic, or recombinantly produced binding partner for a biological molecule or another target of interest. Exemplary binding domains include single chain antibody variable regions (e.g., domain antibodies, sFv, scFv, Fab, Fab′, F(ab′)2, Fv), receptor ectodomains (e.g., RON), or ligands (e.g., cytokines, chemokines). A variety of assays are known for identifying binding domains of the present disclosure that specifically bind a particular target, including Western blot, ELISA, and Biacore analysis.

A binding domain (or a polypeptide comprising a binding domain) “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or polypeptides comprising binding domains) may be classified as “high affinity” binding domains and “low affinity” binding domains. “High affinity” binding domains (or polypeptides comprising binding domains) refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M. “Low affinity” binding domains (or polypeptides comprising binding domains) refer to those binding domains with a Kₐ of up to 10⁷ M⁻¹, up to 10⁶ M⁻¹, up to 10⁵ M. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

An “immunoglobulin binding polypeptide” or “immunoglobulin binding protein” as used herein, refers to a polypeptide that comprises at least one immunoglobulin region, such as a VL, VH, CL, CH1, CH2, CH3, and CH4 domain. The immunoglobulin region may be a wild type immunoglobulin region or an altered immunoglobulin region. Exemplary immunoglobulin binding polypeptides include single chain variable antibody fragment (scFv) (see, e.g., Huston et al., Proc. Natl. Acad. Sci. USA 85: 5879-83, 1988), small modular immunopharmaceutical (SMIP™) proteins (see, U.S. Patent Publication Nos. 2003/0133939, 2003/0118592, and 2005/0136049), PIMS proteins (see, PCT Application Publication No. WO 2009/023386), and multi-functional binding proteins (such as SCORPION™ and Xceptor fusion proteins) (see, for instance, PCT Application Publication No. WO 2007/146968, U.S. Patent Application Publication No. 2006/0051844, and U.S. Pat. No. 7,166,707).

The immunoglobulin binding polypeptides of the invention comprise at least one RON binding domain. Multiple immunoglobulin binding polypeptide constructs are disclosed herein including, for instance, an antibody construct, a SMIP™ protein construct, a SCORPION/Xceptor construct and a heterodimer construct. Unless specifically stated otherwise, the terms “immunoglobulin binding polypeptide,” “binding polypeptide,” “binding domain polypeptide,” “fusion protein,” “fusion polypeptide,” “immunoglobulin-derived fusion protein,” and “RON binding polypeptide” should be considered to be interchangeable.

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

For example, the terms “VL” and “VH” refer to the variable binding region from an antibody light and heavy chain, respectively. The variable binding regions are made up of discrete, well-defined sub-regions known as “complementarity determining regions” (CDRs) and “framework regions” (FRs). The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 of the heavy chain linked to the light chain via an inter-chain disulfide bond.

As used herein, “an Fc region constant domain portion” or “Fc region portion” refers to the heavy chain constant region segment of the Fc fragment (the “fragment crystallizable” region or Fc region) from an antibody, which can include one or more constant domains, such as CH2, CH3, CH4, or any combination thereof. In certain embodiments, an Fc region portion includes the CH2 and CH3 domains of an IgG, IgA, or IgD antibody and any combination thereof, or the CH3 and CH4 domains of an IgM or IgE antibody and any combination thereof. In one embodiment, the CH2CH3 or the CH3CH4 structures are from the same antibody isotype, such as IgG, IgA, IgD, IgE, or IgM. By way of background, the Fc region is responsible for the effector functions of an immunoglobulin, such as ADCC (antibody-dependent cell-mediated cytotoxicity), ADCP (antibody-dependent cellular phagocytosis), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature, 337:525 (1989)). In certain embodiments, an Fc region portion found in polypeptide heterodimers of the present disclosure will be capable of mediating one or more of these effector functions.

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2.

According to crystallographic studies, an IgG hinge domain can be functionally and structurally subdivided into three regions: the upper, the core or middle, and the lower hinge regions (Shin et al., Immunological Reviews 130:87 (1992)). Exemplary upper hinge regions include EPKSCDKTHT (SEQ ID NO:194) as found in IgG1, ERKCCVE (SEQ ID NO:195) as found in IgG2, ELKTPLGDTT HT (SEQ ID NO:196) or EPKSCDTPPP (SEQ ID NO:197) as found in IgG3, and ESKYGPP (SEQ ID NO:198) as found in IgG4. Exemplary middle or core hinge regions include CPPCP (SEQ ID NO:199) as found in IgG1 and IgG2, CPRCP (SEQ ID NO:200) as found in IgG3, and CPSCP (SEQ ID NO:201) as found in IgG4. While IgG1, IgG2, and IgG4 antibodies each appear to have a single upper and middle hinge, IgG3 has four in tandem—one being ELKTPLGDTTHTCPRCP (SEQ ID NO:202) and three being EPKSCDTPPP CPRCP (SEQ ID NO:203).

IgA and IgD antibodies appear to lack an IgG-like core region, and IgD appears to have two upper hinge regions in tandem (see SEQ ID NOS:204 and 205). Exemplary wild type upper hinge regions found in IgA1 and IgA2 antibodies are set forth in SEQ ID NOS:206 and 207.

IgE and IgM antibodies, in contrast, lack a typical hinge region and instead have a CH2 domain with hinge-like properties. Exemplary wild-type CH2 upper hinge-like sequences of IgE and IgM are set forth in SEQ ID NO:208 (VCSRDFTPPTVKILQSSSDGGGHFPPTIQLLCLVSGYTPGTINITWLEDG QVMDVDLSTASTTQEGELASTQSELTLSQKHWLSDRTYTCQVTYQGHTFE DSTKKCA) and SEQ ID NO:209 (VIAELPPKVSVFVPPRDGFFGNPRKSKLIC QATGFSPRQIQVSWLREGKQVGSGVTTDQVQAEAKESGPTTYKVTSTLTI KESDWLGQSMFTCRVDHRGLTFQQNASSMCVP), respectively.

As used herein, a “hinge region” or a “hinge” refers to (a) an immunoglobulin hinge region (made up of, for example, upper and core regions) or a functional variant thereof, including wild type and altered immunoglobulin hinges, (b) a lectin interdomain region or a functional variant thereof, (c) a cluster of differentiation (CD) molecule stalk region or a functional variant thereof, or (d) a portion of a cell surface receptor (interdomain region) that connects immunoglobulin V-like or immunoglobulin C-like domains.

As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and in certain particular embodiments, comprises a human IgG hinge region. Exemplary human wild type immunoglobulin hinge regions are set forth in SEQ ID NOS:206 (IgA1 hinge), 207 (IgA2 hinge), 210 (IgD hinge), 211 (IgG1 hinge), 212 (IgG2 hinge), 213 (IgG3 hinge) and 214 (IgG4 hinge).

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as set forth in SEQ ID NOS:199-201. In certain embodiments, one or more cysteine residues in a wild type or altered immunoglobulin hinge region may be substituted by one or more other amino acid residues (e.g., serine, alanine). In further embodiments, an altered immunoglobulin hinge region may alternatively or additionally have a proline residue substituted by another amino acid residue (e.g., serine, alanine). Exemplary altered wild type immunoglobulin hinge regions include those as set forth in SEQ ID NOS:215-227.

In certain embodiments, there may be one or more (e.g., about 2-8) amino acid residues between the hinge and the Fc region portion due to construct design of fusion polypeptides (e.g., amino acid residues resulting from the use of a restriction enzyme site during the construction of a nucleic acid molecule encoding a fusion polypeptides). As described herein, such amino acid residues may be referred to as “junction amino acids” or “junction amino acid residues.” Exemplary junction amino acids are shown in the hinge variant sequences provided in SEQ ID NOS:14-17 (e.g., in SEQ ID NO:14, the C-terminal SG residues are considered junction amino acids; in SEQ ID NO:15, the N-terminal SS residues are considered junctional residues; in SEQ ID NO:16, the N-terminal SS and the C-terminal SG residues are considered junction amino acids; in SEQ ID NO:17, the N-terminal RT and the C-terminal SG are junction amino acids).

In certain embodiments, junction amino acids are present between an Fc region portion that comprises CH2 and CH3 domains and a heterodimerization domain (CH1 or CL). These junction amino acids are also referred to as a “linker between CH3 and CH1 or CL” if they are present between the C-terminus of CH3 and the N-terminus of CH1 or CL. Such a linker may be, for instance, about 2-1012 amino acids in length. In certain embodiments, the Fc region portion comprises human IgG1 CH2 and CH3 domains in which the C-terminal lysine residue of human IgG1 CH3 is deleted. Exemplary linkers between CH3 and CH1 include those set forth in SEQ ID NO:799-801. Exemplary linkers between CH3 and Cκ include those set forth in SEQ ID NOS:802-804 (in which the carboxyl terminal arginine in the linkers may alternatively be regarded as the first arginine of Cκ). In certain embodiments, the presence of such linkers or linker pairs (e.g., SEQ ID NO:799 as a CH3-CH1 linker in one single chain polypeptide of a heterodimer and SEQ ID NO:802 as a CH3-Cκ linker in the other single chain polypeptide of the heterodimer; SEQ ID NO:800 as a CH3-CH1 linker and SEQ ID NO:803 as a CH3-Cκ linker; and SEQ ID NO:801 as a CH3-CH1 linker and SEQ ID NO:804 as a CH3-Cκ linker) improves the production of heterodimer as compared to the presence of a reference linker, such as the reference KSR sequence as set forth in SEQ ID NO:798 in both single chain polypeptides of a heterodimer.

A “peptide linker” or “variable domain linker” refers to an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a variable domain linker is comprised of about five to about 35 amino acids and in certain embodiments, comprises about 15 to about 25 amino acids.

A “wild type immunoglobulin region” or “wild type immunoglobulin domain” refers to a naturally occurring immunoglobulin region or domain (e.g., a naturally occurring VL, VH, hinge, CL, CH1, CH2, CH3, or CH4) from various immunoglobulin classes or subclasses (including, for example, IgG1, IgG2, IgG3, IgG4, IgA1, IgA2, IgD, IgE, and IgM) and from various species (including, for example, human, sheep, mouse, rat, and other mammals). Exemplary wild type human CH1 regions are set forth in SEQ ID NOS:20, 228-235, wild type human Cκ region in SEQ ID NO:236, wild type human CA regions in SEQ ID NO:237-240, wild type human CH2 domains in SEQ ID NOS:241-249, wild type human CH3 domains in SEQ ID NOS:250-258, and wild type human CH4 domains in SEQ ID NO:259-260.

An “altered immunoglobulin region” or “altered immunoglobulin domain” refers to an immunoglobulin region with a sequence identity to a wild type immunoglobulin region or domain (e.g., a wild type VL, VH, hinge, CL, CH1, CH2, CH3, or CH4) of at least about 75% (e.g., about 80%, 82%, 84%, 86%, 88%, 90%, 91%, 92%, 93%, 94%, 95%, 96%, 97%, 98%, 99%, or 99.5%). For example, an “altered immunoglobulin CH1 region” or “altered CH1 region” refers to a CH1 region with a sequence identity to a wild type immunoglobulin CH1 region (e.g., a human CH1) of at least about 75% (e.g., about 80%, 82%, 84%, 86%, 88%, 90%, 91%, 92%, 93%, 94%, 95%, 96%, 97%, 98%, 99%, or 99.5%). Similarly, an “altered immunoglobulin CH2 domain” or “altered CH2 domain” refers to a CH2 domain with a sequence identity to a wild type immunoglobulin CH1 region (e.g., a human CH2) of at least about 75% (e.g., about 80%, 82%, 84%, 86%, 88%, 90%, 91%, 92%, 93%, 94%, 95%, 96%, 97%, 98%, 99%, or 99.5%).

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values are generated by the NCBI BLAST2.0 software as defined by Altschul et al. (1997) “Gapped BLAST and PSI-BLAST: a new generation of protein database search programs,” Nucleic Acids Res. 25:3389-3402, with the parameters set to default values.

In certain embodiments, an altered immunoglobulin domain only contains conservative amino acid substitutions of a wild type immunoglobulin domain. In certain other embodiments, an altered immunoglobulin domain only contains non-conservative amino acid substitutions of a wild type immunoglobulin domain. In yet other embodiments, an altered immunoglobulin domain contains both conservative and non-conservative amino acid substitutions.

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp. 71-77; Lewin, Genes IV, Oxford University Press, NY and Cell Press, Cambridge, Mass. (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation. Generally, a “derivative” differs from an “analogue” in that a parent polypeptide may be the starting material to generate a “derivative,” whereas the parent polypeptide may not necessarily be used as the starting material to generate an “analogue.” A derivative may have different chemical, biological or physical properties of the parent polypeptide. For example, a derivative may be more hydrophilic or it may have altered reactivity (e.g., a CDR having an amino acid change that alters its affinity for a target) as compared to the parent polypeptide.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

A “receptor” is a protein molecule present in the plasma membrane or in the cytoplasm of a cell to which a signal molecule (i.e., a ligand, such as a hormone, a neurotransmitter, a toxin, a cytokine) may attach. The binding of the single molecule to the receptor results in a conformational change of the receptor, which ordinarily initiates a cellular response. However, some ligands merely block receptors without inducing any response (e.g., antagonists). Some receptor proteins are peripheral membrane proteins, many hormone and neurotransmitter receptors are transmembrane proteins that embedded in the phospholipid bilayer of cell membranes, and another major class of receptors are intracellular proteins such as those for steroid and intracrine peptide hormone receptors.

The term “biological sample” includes a blood sample, biopsy specimen, tissue explant, organ culture, biological fluid (e.g., serum, urine, CSF) or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid sequences, somatic cell hybrid cell lines, immortalized or immortalizable cell lines, differentiated or differentiatable cell lines, transformed cell lines, or the like. In further embodiments of this disclosure, a subject or biological source may be suspected of having or being at risk for having a disease, disorder or condition, including a malignant disease, disorder or condition or a B cell disorder. In certain embodiments, a subject or biological source may be suspected of having or being at risk for having a hyperproliferative, inflammatory, or autoimmune disease, and in certain other embodiments of this disclosure the subject or biological source may be known to be free of a risk or presence of such disease, disorder, or condition.

“Treatment,” “treating” or “ameliorating” refers to either a therapeutic treatment or prophylactic/preventative treatment. A treatment is therapeutic if at least one symptom of disease in an individual receiving treatment improves or a treatment may delay worsening of a progressive disease in an individual, or prevent onset of additional associated diseases.

A “therapeutically effective amount (or dose)” or “effective amount (or dose)” of a specific binding molecule or compound refers to that amount of the compound sufficient to result in amelioration of one or more symptoms of the disease being treated in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formuation or concurrently in separate formulations).

The term “pharmaceutically acceptable” refers to molecular entities and compositions that do not produce allergic or other serious adverse reactions when administered using routes well known in the art.

A “patient in need” refers to a patient at risk of, or suffering from, a disease, disorder or condition that is amenable to treatment or amelioration with an immunoglobulin binding polypeptide or a composition thereof provided herein.

The term “immunoglobulin-derived fusion protein,” as used herein, refers to a fusion protein that comprises at least one immunoglobulin region, such as a VL, VH, CL, CH1, CH2, CH3, and CH4 domain. The immunoglobulin region may be a wild type immunoglobulin region or an altered immunoglobulin region.

Additional definitions are provided throughout the present disclosure.

### Constructs Comprising Binding Domains

The present disclosure provides polypeptides comprising binding domains, in particular, binding domains that specifically bind RON. The polypeptides comprising binding domains of this disclosure may be fusion proteins comprising the binding domains as described herein and further comprising any of a variety of other components/domains such as Fc region domains, linkers, hinges, dimerization/heterodimerization domains, junctional amino acids, tags etc. These components of the immunoglobulin polypeptides are described in further detail below.

Additionally, the immunoglobulin binding polypeptides disclosed herein may be in the form of an antibody or a fusion protein of any of a variety of different formats (e.g., the fusion protein may be in the form of a SMIP™, a PIMS, a Scorpion™/Xceptor protein or an Interceptor protein).

Binding Domains

As indicated above, an immunoglobulin binding polypeptide of the present disclosure comprises a binding domain that specifically binds a target (e.g., RON). Binding of a target by the binding domain may block the interaction between the target (e.g., a receptor such as RON or a ligand) and another molecule, and thus interfere, reduce or eliminate certain functions of the target (e.g., signal transduction).

It should be noted that the primary target of the immunoglobulin binding polypeptides of this disclosure is the RON protein. However, in certain embodiments, the immunoglobulin binding polypeptides may comprise one or more additional binding domains that bind RON, or a target other than RON (e.g., heterologous target). These heterologous target molecules may comprise, for example, a particular cytokine or a molecule that targets the binding domain polypeptide to a particular cell type, a toxin, an additional cell receptor, an antibody, etc.

In certain embodiments, a binding domain, for instance, as part of an Interceptor molecule, may comprise a TCR binding domain for recruitment of T cells to target cells expressing RON (see e.g., Example 8). In certain embodiments, a polypeptide heterodimer as described herein may comprise a binding domain that specifically binds a TCR complex or a component thereof (e.g., TCRα, TCRβ, CD3γ, CD3δ, and CD3ε) and another binding domain that specifically binds to RON.

Thus, a binding domain may be any peptide that specifically binds a target of interest (e.g., RON). Sources of binding domains include antibody variable regions from various species (which can be formatted as antibodies, sFvs, scFvs, Fabs, or soluble V_{H} domain or domain antibodies), including human, rodent, avian, and ovine. Domain antibodies (dAbs) comprise a variable region of a heavy or light chain of an immunoglobulins (V_{H} and V_{L}, respectively) (Holt et al., (2003) Trends Biotechnol. 21:484-490). Additional sources of binding domains include variable regions of antibodies from other species, such as camelid (from camels, dromedaries, or llamas; Ghahroudi et al. (1997) FEBS Letters 414(3):521-526; Vincke et al. (2009) Journal of Biological Chemistry (2009) 284:3273-3284; Hamers-Casterman et al. (1993) Nature, 363:446 and Nguyen et al. (1998) J. Mol. Biol., 275:413), nurse sharks (Roux et al. (1998) Proc. Nat'l. Acad. Sci. (USA) 95:11804), spotted ratfish (Nguyen et al. (2002) Immunogenetics, 54:39), or lamprey (Herrin et al., (2008) Proc. Nat'l. Acad. Sci. (USA) 105:2040-2045 and Alder et al. (2008) Nature Immunology 9:319-327). These antibodies can apparently form antigen-binding regions using only heavy chain variable region, i.e., these functional antibodies are homodimers of heavy chains only (referred to as “heavy chain antibodies”) (Jespers et al. (2004) Nature Biotechnology 22:1161-1165; Cortez-Retamozo et al. (2004) Cancer Research 64:2853-2857; Baral et al. (2006) Nature Medicine 12:580-584, and Barthelemy et al. (2008) Journal of Biological Chemistry 283:3639-3654).

An alternative source of binding domains of this disclosure includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), ankyrin repeat proteins (Binz et al. (2003) Journal of Molecular Biology 332:489-503 and Binz et al. (2004) Nature Biotechnology 22(5):575-582), fibronectin binding domains (Richards et al. (2003) Journal of Molecular Biology 326:1475-1488; Parker et al. (2005) Protein Engineering Design and Selection 18(9):435-444 and Hackel et al. (2008) Journal of Molecular Biology 381:1238-1252), cysteine-knot miniproteins (Vita et al. (1995) Proc. Nat'l. Acad. Sci. (USA) 92:6404-6408; Martin et al. (2002) Nature Biotechnology 21:71-76 and Huang et al. (2005) Structure 13:755-768), tetratricopeptide repeat domains (Main et al. (2003) Structure 11:497-508 and Cortajarena et al. (2008) ACS Chemical Biology 3:161-166), leucine-rich repeat domains (Stumpp et al. (2003) Journal of Molecular Biology 332:471-487), lipocalin domains (see, e.g., WO 2006/095164, Beste et al. (1999) Proc. Nat'l. Acad. Sci. (USA) 96:1898-1903 and Schonfeld et al. (2009) Proc. Nat'l. Acad. Sci. (USA) 106:8198-8203), V-like domains (see, e.g., U.S. Patent Application Publication No. 2007/0065431), C-type lectin domains (Zelensky and Gready (2005) FEBS J. 272:6179; Beavil et al. (1992) Proc. Nat'l. Acad. Sci. (USA) 89:753-757 and Sato et al. (2003) Proc. Nat'l. Acad. Sci. (USA) 100:7779-7784), mAb² or Fcab™ (see, e.g., PCT Patent Application Publication Nos. WO 2007/098934; WO 2006/072620), or the like (Nord et al. (1995) Protein Engineering 8(6):601-608; Nord et al. (1997) Nature Biotechnology 15:772-777; Nord et al. (2001) European Journal of Biochemistry 268(15):4269-4277 and Binz et al. (2005) Nature Biotechnology 23:1257-1268).

Binding domains of this disclosure can be generated as described herein or by a variety of methods known in the art (see, e.g., U.S. Pat. Nos. 6,291,161 and 6,291,158). For example, binding domains of this disclosure may be identified by screening a Fab phage library for Fab fragments that specifically bind to a target of interest (see Hoet et al. (2005) Nature Biotechnol. 23:344). Additionally, traditional strategies for hybridoma development using a target of interest as an immunogen in convenient systems (e.g., mice, HUMAb MOUSE®, TC MOUSE™, KM-MOUSE®, llamas, chicken, rats, hamsters, rabbits, etc.) can be used to develop binding domains of this disclosure.

In some embodiments, a binding domain is a single chain Fv fragment (scFv) that comprises V_{H} and V_{L} regions specific for a target of interest. In certain embodiments, the V_{H} and V_{L} domains are human. Exemplary V_{L} and V_{H} regions include the V_{L} and V_{H} regions from the 4C04 and 11H09 antibodies as described herein. The light chain amino acid sequence of the 4C04 is set forth in SEQ ID NO:152, and its CDR1, CDR2, and CDR3 as set forth in SEQ ID NOS:141-143, respectively. The heavy chain amino acid sequence of the 4C04 is set forth in SEQ ID NO:153, and its CDR1, CDR2, and CDR3 are set forth in SEQ ID NOS:144-146, respectively. The light chain amino acid sequence of the 11H09 scFv is set forth in SEQ ID NO:80, and its CDR1, CDR2, and CDR3 are set forth in SEQ ID NOS:67-69, respectively. The heavy chain amino acid sequence of the 11H09 scFv is set forth in SEQ ID NO:81, and its CDR1, CDR2, and CDR3 are set forth in SEQ ID NOS:70-72, respectively.

In certain embodiments, a binding domain comprises or is a sequence that is at least about 90%, at least about 91%, at least about 92%, at least about 93%, at least about 94%, at least about 95%, at least about 96%, at least about 97%, at least about 98%, at least about 99%, at least about 99.5%, or 100% identical to an amino acid sequence of a light chain variable region (V_{L}) (e.g., SEQ ID NOS: 80 and 152) or to a heavy chain variable region (V_{H}) (e.g., SEQ ID NOS:81 and 153), or both. In certain embodiments, each CDR comprises no more than one, two, or three substitutions, insertions or deletions, as compared to that from a monoclonal antibody or fragment or derivative thereof that specifically binds to a target of interest (e.g., RON). In further embodiments, a binding domain comprises a CDR1, CDR2 and CDR3 (e.g., CDR1, CDR2 and CDR3 from the 4C04 and 11H09 antibodies as described herein) wherein one, two, or three of the CDRs comprise a fragment of a CDR as disclosed herein, such as a fragment of a CDR having 3, 4, 5, 6, 7, 8, or 9 amino acids of a CDR described herein.

In certain embodiments, a binding domain comprises or is a sequence that is a humanized version of a light chain variable region (V_{L}) (e.g., SEQ ID NOS: 80 and 152) or a heavy chain variable region (V_{H}) (e.g., SEQ ID NOS:81 and 153), or both. Exemplary humanized light chain variable regions (V_{L}) are provided in SEQ ID NOS:82, 83 and 154. Exemplary humanized heavy chain variable regions (V_{H}) are provided in SEQ ID NOS:84-86 and 155-156.

In certain embodiments, a binding domain V_{H} region of the present disclosure can be derived from or based on a V_{H} of a known monoclonal antibody (e.g., DX07 anti-RON antibody) and contains about one or more (e.g., about 2, 3, 4, 5, 6, 7, 8, 9, 10) insertions, about one or more (e.g., about 2, 3, 4, 5, 6, 7, 8, 9, 10) deletions, about one or more (e.g., about 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. The insertion(s), deletion(s) or substitution(s) may be anywhere in the VH region, including at the amino- or carboxyl-terminus or both ends of this region, provided that each CDR comprises zero changes or at most one, two, or three changes and provided a binding domain containing the modified VH region can still specifically bind its target with an affinity similar to the wild type binding domain.

In further embodiments, a VL region in a binding domain of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. The insertion(s), deletion(s) or substitution(s) may be anywhere in the VL region, including at the amino- or carboxyl-terminus or both ends of this region, provided that each CDR comprises zero changes or at most one, two, or three changes and provided a binding domain containing the modified V_{L} region can still specifically bind its target with an affinity similar to the wild type binding domain.

The VH and VL domains may be arranged in either orientation (i.e., from amino-terminus to carboxy terminus, VH-VL or VL-VH) and may optionally be joined by a variable domain linker, e.g., an amino acid sequence (e.g., having a length of about five to about 35 amino acids) capable of providing a spacer function such that the two sub-binding domains can interact to form a functional binding domain. In certain embodiments, an amino acid sequence that joins the VH and VL domains (also referred to herein as a “variable domain linker”) includes those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. In certain embodiments, the linker is GGGGSGGGGS GGGGS (SEQ ID NO:179) or GGGGSGGGGS GGGGSGGGGS (SEQ ID NO:180). In certainembodiments, these (GlyₙSer)-based linkers are used to link the VH and VL domains in a binding domain, but are not used to link a binding domain to any other domain, e.g., a heterodimerization domain or to an Fc region portion.

Exemplary binding domains specific for RON include a 4C04 scFv as set forth in SEQ ID NO:157, or humanized versions thereof as provided in SEQ ID NOS:158 and 159, and a 11H09 scFv as set forth in SEQ ID NO:87 or humanized versions thereof as provided in SEQ ID NO:88-93.

The light chain amino acid sequence of the 4C04 scFv is set forth in SEQ ID NO:152, and its CDR1, CDR2, and CDR3 are set forth in SEQ ID NOS:141-143, respectively. The heavy chain amino acid sequence of the 4C04 scFv is set forth in SEQ ID NO:153, and its CDR1, CDR2, and CDR3 are set forth in SEQ ID NOS:144-146, respectively.

The light chain amino acid sequence of the 11H09 scFv is set forth in SEQ ID NO:80, and its CDR1, CDR2, and CDR3 are set forth in SEQ ID NOS:67-69, respectively. The heavy chain amino acid sequence of the 11H09 scFv is set forth in SEQ ID NO:81, and its CDR1, CDR2, and CDR3 are set forth in SEQ ID NOS:70-72, respectively.

In certain embodiments, the RON binding domain comprises the RON ligand macrophage stimulating protein (MSP), or a RON-binding portion thereof. Sequences of the MSP protein are known in the art and available from public databases such as GENBANK. Illustrative amino acid sequences of MSP may be found in GENBANK Accession No. AAA59872 gi398038 (SEQ ID NO:785) and NCBI Reference Sequence NP_{—}066278 as set forth in SEQ ID NO:809. (see also J. Biol. Chem. 268 (21), 15461-15468 (1993)).

A target molecule, which is specifically bound by a binding domain contained in a binding polypeptide or polypeptide heterodimer thereof of the present disclosure, may be found on or in association with a cell of interest (“target cell”). Exemplary target cells include a cancer cells, a cell associated with an autoimmune disease or disorder or with an inflammatory disease or disorder, and an infectious cell (e.g., an infectious bacterium). A cell of an infectious organism, such as a mammalian parasite, is also contemplated as a target cell. A target molecule may also not be associated with a cell. Exemplary target molecules not associated with a cell include soluble proteins, secreted proteins, deposited proteins, and extracellular structural (matrix) proteins.

In certain embodiments, binding domains of the immunoglobulin binding proteins of the present disclosure recognize a target selected from a tumor cell, a monocyte/macrophage cell target, and an epithelial cell. In further embodiments, the binding domains of binding polypeptides of the present disclosure bind a receptor protein, such as peripheral membrane receptor proteins or transmembrane receptor proteins.

In certain embodiments, the immunoglobulin binding proteins of the present disclosure specifically bind RON.

Immunoglobulin Binding Polypeptides with Dimerization/Heterodimerization Domains

In certain embodiments, an immunoglobulin binding polypeptide of the invention may comprise a dimerization or heterodimerization domain. A “polypeptide heterodimer” or “heterodimer,” as used herein, refers to a dimer formed from two different single chain polypeptides.

Dimerization/heterodimerization domains may be used where it is desired to form homo or heterodimers from two single chain polypeptides, where one or both single chain polypeptides comprise a binding domain. It should be noted that in certain embodiments, one single chain polypeptide member of certain heterodimers described herein may not contain a binding domain. See, e.g., RON-f03-f06 Interceptor molecules as summarized in Table 4. These single chain polypeptide members lacking a binding domain may contain any of the components of immunoglobulin binding polypeptides as described herein (e.g., Fc regions, hinges, linkers, dimerization/heterodimerization domains, junctional amino acids, etc).

In certain embodiments, the binding polypeptides comprise a “dimerization domain,” which refers to an amino acid sequence that is capable of promoting the association of at least two single chain polypeptides or proteins via non-covalent or covalent interactions, such as by hydrogen bonding, electrostatic interactions, salt bridges, Van der Waal's forces, disulfide bonds, hydrophobic interactions, or the like, or any combination thereof. Exemplary dimerization domains include immunoglobulin heavy chain constant regions or sub-regions. It should be understood that a dimerization domain can promote the formation of dimers or higher order multimer complexes (such as trimers, tetramers, pentamers, hexamers, septamers, octamers, etc.).

Where heterodimerization is desired, the heterodimerization domains of a polypeptide heterodimer are different from each other and thus may be differentially modified to facilitate heterodimerization of both chains and to minimize homodimerization of either chain. Heterodimerization domains provided herein allow for efficient heterodimerization between different polypeptides and facilitate purification of the resulting polypeptide heterodimers.

As provided herein, heterodimerization domains useful for promoting heterodimerization of two different single chain polypeptides (e.g., one short and one long) according to the present disclosure include immunoglobulin CH1 and CL domains, for instance, human CH1 and CL domains. In certain embodiments, an immunoglobulin heterodimerization domain is a wild type CH1 region, such as a wild type IgG1, IgG2, IgG3, IgG4, IgA1, IgA2 IgD, IgE, or IgM CH1 region. In further embodiments, an immunoglobulin heterodimerization domain is a wild type human IgG1, IgG2, IgG3, IgG4, IgA1, IgA2, IgD, IgE, or IgM CH1 region as set forth in SEQ ID NOS:181-189, respectively. In certain embodiments, an immunoglobulin heterodimerization domain is a wild type human IgG1 CH1 region as set forth in SEQ ID NO:20, which may, in certain embodiments, be used in a construct herein without the terminal “RT” residues.

In further embodiments, an immunoglobulin heterodimerization domain is an altered immunoglobulin CH1 region, such as an altered IgG1, IgG2, IgG3, IgG4, IgA1, IgA2 IgD, IgE, or IgM CH1 region. In certain embodiments, an immunoglobulin heterodimerization domain is an altered human IgG1, IgG2, IgG3, IgG4, IgA1, IgA2, IgD, IgE, or IgM CH1 region. In still further embodiments, a cysteine residue of a wild type CH1 region (e.g., a human CH1) involved in forming a disulfide bond with a wild type immunoglobulin CL domain (e.g., a human CL) is deleted or substituted in the altered immunoglobulin CH1 region such that a disulfide bond is not formed between the altered CH1 region and the wild type CL domain.

In certain embodiments, an immunoglobulin heterodimerization domain is a wild type CL domain, such as a wild type Cκ domain or a wild type CA domain. In particular embodiments, an immunoglobulin heterodimerization domain is a wild type human Cκ or human CA domain as set forth in SEQ ID NOS:190 and 191, respectively. In further embodiments, an immunoglobulin heterodimerization domain is an altered immunoglobulin CL domain, such as an altered Cκ or CA domain, for instance, an altered human Cκ or human CA domain.

In certain embodiments, a cysteine residue of a wild type CL domain (e.g., a human CL) involved in forming a disulfide bond with a wild type immunoglobulin CH1 region (e.g., a human CH1) is deleted or substituted in the altered immunoglobulin CL domain. Such altered CL domains may further comprise an amino acid deletion at their amino termini. An exemplary Cκ domain is set forth in SEQ ID NO:21, in which the first arginine and the last cysteine of the wild type human Ck domain are both deleted. An exemplary CA domain is set forth in SEQ ID NO:192, in which the first arginine of a wild type human CA domain is deleted and the cysteine involved in forming a disulfide bond with a cysteine in a CH1 region is substituted by a serine.

In further embodiments, an immunoglobulin heterodimerization domain is an altered Cκ domain that contains one or more amino acid substitutions, as compared to a wild type Cκ domain, at positions that may be involved in forming the interchain-hydrogen bond network at a Cκ-Cκ interface. For example, in certain embodiments, an immunoglobulin heterodimerization domain is an altered human Cκ domain having one or more amino acids at positions N29, N30, Q52, V55, T56, S68 or T70 that are substituted with a different amino acid. The numbering of the amino acids is based on their positions in the altered human Cκ sequence as set forth in SEQ ID NO:21. In certain embodiments, an immunoglobulin heterodimerization domain is an altered human Cκ domain having one, two, three or four amino acid substitutions at positions N29, N30, V55, or T70. The amino acid used as a substitute at the above-noted positions may be an alanine, or an amino acid residue with a bulk side chain moiety such as arginine, tryptophan, tyrosine, glutamate, glutamine, or lysine. Exemplary altered human Cκ domains are set forth in SEQ ID NOS: 261-297. Examples of altered human Ck domains are provided in SEQ ID NOS:22 and 23 in which amino acid residues 30, 55 and 70 have been modified. These two Ck variants are referred to as Ck (YAE) and Ck (EAE), respectively, referring to the three replacement residues. Certain altered human Cκ domains can facilitate heterodimerization with a CH1 region, but minimize homodimerization with another Cκ domain. Representative altered human Cκ domains are set forth in SEQ ID NOS:298 (N29W V55A T70A), 299 (N29Y V55A T70A), 300 (T70E N29A N30A V55A), 301 (N30R V55A T70A), 302 (N30K V55A T70A), 303 (N30E V55A T70A), 304 (V55R N29A N30A), 305 (N29W N30Y V55A T70E), 306 (N29Y N30Y V55A T70E), 23 (N30E V55A T70E), and 22 (N30Y V55A T70E).

In further embodiments, other altered human Cκ domains include N30D V55A T70E (DAE); N30M V55A T70E (MAE); N30S V55A T70E (SAE); and N30F V55A T70E (FAE).

In further embodiments, specific altered CH1 domains may be appropriately paired with particular altered human Cκ domains to destabilize homodimerization. In this regard, illustrative altered domain pairs include Cκ L29E+CH1 V68K and Cκ L29K+CH1 V68E.

In certain embodiments, in addition to or alternative to the mutations in Ck domains described herein, both the immunoglobulin heterodimerization domains (i.e., immunoglobulin CH1 and CL domains) of a polypeptide heterodimer have mutations so that the resulting immunoglobulin heterodimerization domains form salt bridges (i.e., ionic interactions) between the amino acid residues at the mutated sites. For example, the immunoglobulin heterodimerization domains of a polypeptide heterodimer may be a mutated CH1 domain in combination with a mutated Ck domain. In the mutated CH1 domain, valine at position 68 (V68) of the wild type human CH1 domain is substituted by an amino acid residue having a negative charge (e.g., asprartate or glutamate), whereas leucine at position 29 (L29) of a mutated human Ck domain in which the first arginine and the last cysteine have been deleted is substituted by an amino acid residue having a positive charge (e.g., lysine, arginine or histidine). The charge-charge interaction between the amino acid residue having a negative charge of the resulting mutated CH1 domain and the amino acid residue having a positive charge of the resulting mutated Ck domain forms a salt bridge, which stabilizes the heterodimeric interface between the mutated CH1 and Ck domains. Alternatively, V68 of the wild type CH1 may be substituted by an amino acid residue having a positive charge, whereas L29 of a mutated human Ck domain in which the first arginine and the last cysteine have been deleted may be substituted by an amino acid residue having a negative charge. Exemplary mutated CH1 domains in which V68 is substituted by an amino acid with either a negative or positive charge include V68K and V68E substituted CH1 domains. Exemplary mutated Cκ domains in which L29 is substituted by an amino acid with either a negative or positive charge include L29E and L29K substituted Cκ domains. In certain embodiments, the terminal cysteine residue present in wild type Cκ is deleted.

Positions other than V68 of human CH1 domain and L29 of human Ck domain may be substituted with amino acids having opposite charges to produce ionic interactions between the amino acids in addition or alternative to the mutations in V68 of CH1 domain and L29 of Ck domain. Such positions can be identified by any suitable method, including random mutagenesis, analysis of the crystal structure of the CH1-Ck pair to identify amino acid residues at the CH1-Ck interface, and further identifying suitable positions among the amino acid residues at the CH1-Ck interface using a set of criteria (e.g., propensity to engage in ionic interactions, proximity to a potential partner residue, etc.).

In certain embodiments, where polypeptide heterodimers are desired, the single chain polypeptides used may contain only one pair of heterodimerization domains. For example, a first chain of a polypeptide heterodimer may comprise a CH1 region as a heterodimerization domain, while a second chain may comprise a CL domain (e.g., a Cκ or Cλ) as a heterodimerization domain. Alternatively, a first chain may comprise a CL region (e.g., a Cκ or Cλ) as a heterodimerization domain, while a second chain may comprise a CH1 region as a heterodimerization domain. As set forth herein, the heterodimerization domains of the first and second chains are capable of associating to form a polypeptide heterodimer of this disclosure.

In certain other embodiments, immunoglobulin binding polypeptides may have two pairs of heterodimerization domains. For example, a first chain of a polypeptide heterodimer may comprise two CH1 regions, while a second chain may have two CL domains that associate with the two CH1 regions in the first chain. Alternatively, a first chain may comprise two CL domains, while a second chain may have two CH1 regions that associate with the two CL domains in the first chain. In certain embodiments, a first chain polypeptide comprises a CH1 region and a CL domain, while a second chain polypeptide comprises a CL domain and a CH1 region that associate with the CH1 region and the CL domain, respectively, of the first chain polypeptide.

In the embodiments where a polypeptide heterodimer comprises only one heterodimerization pair (i.e., one heterodimerization domain in each chain), the heterodimerization domain of each chain may be located amino terminal to the Fc region portion of that chain. Alternatively, the heterodimerization domain in each chain may be located carboxyl terminal to the Fc region portion of that chain.

In the embodiments where a polypeptide heterodimer comprises two heterodimerization pairs (i.e., two heterodimerization domains in each chain), both heterodimerization domains in each chain may be located amino terminal to the Fc region portion of that chain. Alternatively, both heterodimerization domains in each chain may be located carboxyl terminal to the Fc region portion of that chain. In further embodiments, one heterodimerization domain in each chain may be located amino terminal to the Fc region portion of that chain, while the other heterodimerization domain of each chain may be located carboxyl terminal to the Fc region portion of that chain. In other words, in those embodiments, the Fc region portion is interposed between the two heterodimerization domains of each chain.

Fc Region Portion

As indicated herein, the binding constructs of the present disclosure, whether they comprise a binding domain or not, may comprise an Fc region constant domain portion (also referred to as an Fc region portion). The inclusion of an Fc region portion slows clearance of the binding proteins from circulation after administration to a subject. By mutations or other alterations, the Fc region portion further enables relatively easy modulation of effector functions of the binding polypeptide, or dimers or heterodimers thereof, (e.g., ADCC, ADCP, CDC, complement fixation and binding to Fc receptors), which can either be increased or decreased depending on the disease being treated, as known in the art and described herein. In certain embodiments, an Fc region portion of binding polypeptides of the present disclosure will be capable of mediating one or more of these effector functions.

An Fc region portion present in single chain polypeptides may comprise a CH2 domain, a CH3 domain, a CH4 domain or any combination thereof. For example, an Fc region portion may comprise a CH2 domain, a CH3 domain, both CH2 and CH3 domains, both CH3 and CH4 domains, two CH3 domains, a CH4 domain, or two CH4 domains.

A CH2 domain that may form an Fc region portion of a single chain polypeptide of the present disclosure may be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:241, 246-248 and 242-244, respectively. In certain embodiments, the CH2 domain is a wild type human IgG1 CH2 domain as set forth in SEQ ID NO:241.

In certain embodiments, a CH2 domain is an altered immunoglobulin CH2 region (e.g., an altered human IgG1 CH2 domain) that comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO:307.

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region may comprise one or more (e.g., two, three, four or five) amino acid deletions at positions 234-238, for instance, a deletion at one of position 236 or position 237 while the other position is substituted. The above-noted mutation(s) decrease or eliminate the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region may comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, an mutated CH2 region (e.g., an altered human IgG1 CH2 domain) in a fusion protein of the present disclosure may contain one or more (e.g., 2, 3, 4, 5, or 6) additional amino acid substitutions (e.g., substituted with alanine) at one or more positions involved in complement fixation (e.g., at positions I253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322 (SEQ ID NO:308).

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. In one embodiment, the amino acids at the above-noted positions are substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO:309), human IgG2 CH2 region with alanine substitutions at V234, G236, and N297 (SEQ ID NO:310), human IgG4 CH2 region with alanine substitutions at F234, L235, G237 and N297 and a deletion of G236 (SEQ ID NO:311), human IgG4 CH2 region with alanine substitutions at F234 and N297 (SEQ ID NO:312), human IgG4 CH2 region with alanine substitutions at L235 and N297 (SEQ ID NO:313), human IgG4 CH2 region with alanine substitutions at G236 and N297 (SEQ ID NO:314), and human IgG4 CH2 region with alanine substitutions at G237 and N297 (SEQ ID NO:315).

In certain embodiments, in addition to the amino acid substitutions described above, an altered CH2 region (e.g., an altered human IgG1 CH2 domain) may contain one or more additional amino acid substitutions at one or more positions other than the above-noted positions. Such amino acid substitutions may be conservative or non-conservative amino acid substitutions. For example, in certain embodiments, P233 may be changed to E233 in an altered IgG2 CH2 region (see, e.g., SEQ ID NO:310). In addition or alternatively, in certain embodiments, the altered CH2 region may contain one or more amino acid insertions, deletions, or both. The insertion(s), deletion(s) or substitution(s) may anywhere in an immunoglobulin CH2 region, such as at the N- or C-terminus of a wild type immunoglobulin CH2 region resulting from linking the CH2 region with another region (e.g., a binding domain or a heterodimerization domain) via a hinge.

In certain embodiments, an altered CH2 region in a polypeptide heterodimer of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a polypeptide heterodimer of the present disclosure may be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure may be derived from a CH2 region of human IgG1, IgG2 or IgG4, or mouse IgG2a (e.g., IGHG2c), whose sequences are set forth in SEQ ID NOS:241, 246, 248 and 316.

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:317), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:318), and optionally an N297 mutation (e.g., to alanine).

In certain embodiments, an altered CH2 domain is an altered human IgG1 CH2 domain with mutations known in the art that enhance immunological activities such as ADCC, ADCP, CDC, complement fixation, Fc receptor binding, or any combination thereof.

The CH3 domain that may form an Fc region portion of a binding polypeptide of the present disclosure may be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:319-328, respectively. In certain embodiments, the CH3 domain is a wild type human IgG1 CH3 domain as set forth in SEQ ID NO:319. In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain may be a human IgG1 CH3 domain with one or two mutations at positions H433 and N434 (positions are numbered according to EU numbering). The mutations in such positions may be involved in complement fixation. In certain other embodiments, an altered CH3 domain may be a human IgG1 CH3 domain but with one or two amino acid substitutions at position F405 or Y407. The amino acids at such positions are involved in interacting with another CH3 domain. In certain embodiments, an altered CH3 domain may be an altered human IgG1 CH3 domain with its last lysine deleted. The sequence of this altered CH3 domain is set forth in SEQ ID NO:329.

In certain embodiments, particularly where a polypeptide heterodimer is desired, the polypeptides of the heterodimer comprise a CH3 pair that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations may be introduced into each of the two CH3 domains so that the steric complementarity required for CH3/CH3 association obligates these two CH3 domains to pair with each other. For example, a CH3 domain in one single chain polypeptide of a polypeptide heterodimer may contain a T366W mutation (a “knob” mutation, which substitutes a small amino acid with a larger one), and a CH3 domain in the other single chain polypeptide of the polypeptide heterodimer may contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that may form an Fc region portion of a single chain polypeptide, which may or may not contain a binding domain, may be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:330 and 331, respectively. In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an Fc region constant domain portion comprises a combination of CH2, CH3 or CH4 domains (i.e., more than one constant sub-domain selected from CH2, CH3 and CH4). For example, the Fc region portion may comprise CH2 and CH3 domains or CH3 and CH4 domains. In certain other embodiments, the Fc region portion may comprise two CH3 domains and no CH2 or CH4 domains (i.e., only two or more CH3). The multiple constant sub-domains that form an Fc region portion may be based on or derived from the same immunoglobulin molecule, or the same class or subclass immunoglobulin molecules. In certain embodiments, the Fc region portion is an IgG CH2CH3 (e.g., IgG1 CH2CH3, IgG2 CH2CH3, and IgG4 CH2CH3) and in certain embodiments is human (e.g., human IgG1, IgG2, and IgG4) CH2CH3. For example, in certain embodiments, the Fc region portion comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted.

Alternatively, the multiple constant sub-domains may be based on or derived from different immunoglobulin molecules, or different classes or subclasses immunoglobulin molecules. For example, in certain embodiments, an Fc region portion comprises both human IgM CH3 domain and human IgG1 CH3 domain. The multiple constant sub-domains that form an Fc region portion may be directly linked together or may be linked to each other via one or more (e.g., 2-8) amino acids.

Exemplary Fc region portions are set forth in SEQ ID NOS:18-19, 332-341.

With regard to heterodimers as disclosed herein, in certain embodiments, the Fc region portions of both single chain polypeptides of a polypeptide heterodimer are identical to each other. In certain other embodiments, the Fc region portion of one single chain polypeptide of a polypeptide heterodimer is different from the Fc region portion of the other single chain polypeptide of the heterodimer. For example, one Fc region portion may contain a CH3 domain with a “knob” mutation, whereas the other Fc region portion may contain a CH3 domain with a “hole” mutation.

Hinges

A hinge region contained in any of the immunoglobulin binding polypeptides described herein, e.g., single chain polypeptides, with or without binding domains, according to the present disclosure may be located (a) immediately amino terminal to an Fc region portion (e.g., depending on the isotype, amino terminal to a CH2 domain wherein the Fc region portion is a CH2CH3, or amino terminal to a CH3 domain wherein the Fc region portion is a CH3CH4), (b) interposed between and connecting a binding domain (e.g., scFv) and a heterodimerization domain, (c) interposed between and connecting a heterodimerization domain and an Fc region portion (e.g., wherein the Fc region portion is a CH2CH3 or a CH3CH4, depending on the isotype or isotypes), (d) interposed between and connecting an Fc region portion and a binding domain, (e) at the amino terminus of the single chain polypeptide, or (f) at the carboxyl terminus of the single chain polypeptide.

In certain embodiments, a hinge is a wild type human immunoglobulin hinge region (e.g., human immunoglobulin hinge regions as set forth in SEQ ID NOS:342-348). In certain other embodiments, one or more amino acid residues may be added at the amino- or carboxyl-terminus of a wild type immunoglobulin hinge region as part of a fusion protein construct design. For example, additional junction amino acid residues at the hinge amino-terminus can be “RT,” “RSS,” “SS”, “TG,” or “T”, or at the hinge carboxyl-terminus can be “SG”, or a hinge deletion can be combined with an addition, such as ΔP with “SG” added at the carboxyl terminus. Illustrative variant hinges are provided in SEQ ID NOS:14-17.

In certain embodiments, a hinge is an altered immunoglobulin hinge in which one or more cysteine residues in a wild type immunoglobulin hinge region is substituted with one or more other amino acid residues (e.g., serine or alanine). For example, a hinge may be an altered immunoglobulin hinge based on or derived from a wild type human IgG1 hinge as set forth in SEQ ID NO:349, which from amino terminus to carboxyl terminus comprises the upper hinge region (EPKSCDKTHT, SEQ ID NO:194) and the core hinge region (CPPCP, SEQ ID NO:199). Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine). An altered immunoglobulin hinge may additionally have a proline substituted with another amino acid (e.g., serine or alanine). For example, the above-described altered human IgG1 hinge may additionally have a proline located carboxyl terminal to the three cysteines of wild type human IgG1 hinge region substituted by another amino acid residue (e.g., serine, alanine). In one embodiment, the prolines of the core hinge region are not substituted. Exemplary altered immunoglobulin hinges are set forth in SEQ ID NOS: 350-377. In one embodiment, an altered IgG1 hinge is an altered human IgG1 hinge in which the first cysteine is substituted by serine. The sequence of this exemplary altered IgG1 hinge is set forth in SEQ ID NO:354, and is referred to as the “human IgG1 SCC-P hinge” or “SCC-P hinge.” In certain embodiments, one or more amino acid residues (e.g., “RT,” “RSS,” or “T”) may be added at the amino- or carboxyl-terminus of a mutated immunoglobulin hinge region as part of a fusion protein construct design.

In certain embodiments, a hinge polypeptide comprises or is a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to a wild type immunoglobulin hinge region, such as a wild type human IgG1 hinge, a wild type human IgG2 hinge, or a wild type human IgG4 hinge.

In further embodiments, a hinge may be a hinge that is not based on or derived from an immunoglobulin hinge (i.e., not a wild type immunoglobulin hinge or an altered immunoglobulin hinge). In one embodiment, these types of non-immunoglobulin based hinges are used on or near the carboxyl end (e.g., located carboxyl terminal to Fc region portions) of the polypeptides described herein. Examples for such hinges include peptides from the interdomain or stalk region of type II C-lectins or CD molecules, such as the stalk regions of CD69, CD72, CD94, NKG2A and NKG2D as set forth in SEQ ID NOS:378-383. Additional exemplary hinges include those as set forth in SEQ ID NOS:384-419.

Alternative hinges that can be used herein are from portions of cell surface receptors (interdomain regions) that connect immunoglobulin V-like or immunoglobulin C-like domains. Regions between Ig V-like domains where the cell surface receptor contains multiple Ig V-like domains in tandem and between Ig C-like domains where the cell surface receptor contains multiple tandem Ig C-like regions are also contemplated as hinges useful in single chain polypeptides of polypeptide heterodimers. In certain embodiments, hinge sequences comprising cell surface receptor interdomain regions may further contain a naturally occurring or added motif, such as an IgG core hinge sequence that confers one or more disulfide bonds to stabilize the polypeptide heterodimer formation. Examples of hinges include interdomain regions between the Ig V-like and Ig C-like regions of CD2, CD4, CD22, CD33, CD48, CD58, CD66, CD80, CD86, CD150, CD166, and CD244.

In certain embodiments, hinge sequences have about 5 to 150 amino acids, about 5 to 10 amino acids, about 10 to 20 amino acids, about 20 to 30 amino acids, about 30 to 40 amino acids, about 40 to 50 amino acids, about 50 to 60 amino acids, about 5 to 60 amino acids, about 5 to 40 amino acids, for instance, about 8 to 20 amino acids or about 12 to 15 amino acids. Hinges may be primarily flexible, but may also provide more rigid characteristics or may contain primarily α-helical structure with minimal β-sheet structure. The lengths or the sequences of the hinges may affect the binding affinities of the binding domains to which the hinges are directly or indirectly (via another region or domain, such as a heterodimerization domain) connected as well as one or more activities of the Fc region portions to which the hinges are directly or indirectly connected.

In certain embodiments, hinge sequences are stable in plasma and serum and are resistant to proteolytic cleavage. The first lysine in the IgG1 upper hinge region may be mutated to minimize proteolytic cleavage. For instance, the lysine may be substituted with methionine, threonine, alanine or glycine, or is deleted (see, e.g., SEQ ID NOS:420-475, which may include junction amino acids at the amino terminus, for instance, RT).

In some embodiments, hinge sequences may contain a naturally occurring or added motif such as an immunoglobulin hinge core structure CPPC (SEQ ID NO:476) that confers the capacity to form a disulfide bond or multiple disulfide bonds to stabilize the carboxyl-terminus of a molecule. In other embodiments, hinge sequences may contain one or more glycosylation sites.

Exemplary hinges, including altered immunoglobulin hinges, are set forth in SEQ ID NOS:389-475 and 477-606. Additional illustrative hinges, including variant hinges, are set forth in SEQ ID NOs:790-797 and 805-506.

In certain embodiments, the immunoglobulin binding polypeptides comprise more than one hinge. For example, a single chain polypeptide having two binding domains, one of which at the amino terminus and the other at the carboxyl terminus, may have two hinges. One hinge may be directly or indirectly (e.g., via a heterodimerization domain) connected to the binding domain at or near the amino terminus, and the other hinge may be connected (e.g., directly connected) to the other binding domain at or near the carboxyl terminus. In certain embodiments, even if a single chain polypeptide has only one binding domain, it may have more than one hinge, for example, at its amino or carboxyl terminus. In certain embodiments, such as where heterodimeration is desired, such a hinge may interact with a corresponding hinge in a second chain of a heterodimer, such as forming one or more interchain disulfide bonds, to facilitate or enhance heterodimerization of the two chains. A hinge (H-I) of a SCP-I of a polypeptide heterodimer “corresponds to” a hinge (H-II) of a SCP-II of the heterodimer when H-I and H-II are located on the same end of the Fc region portion of their respective single chain polypeptide. For example, a polypeptide heterodimer may comprise the following two single chain polypeptides: A first chain polypeptide from amino to carboxyl terminus comprises a first binding domain, CH1, hinge, CH2, and CH3, and a second chain polypeptide from amino to carboxyl terminus comprises a CK, first hinge, CH2, CH3, second hinge, and a second binding domain. The hinge in the first chain would be regarded as “corresponding” to the first hinge of the second chain because both are amino terminal to the Fc region portions to which they are connected.

In certain embodiments, particularly where an immunoglobulin binding polypeptide comprises a binding domain at or near its carboxyl terminus, a hinge may be present to link the binding domain with another portion of the polypeptide (e.g., an Fc region portion or a heterodimerization domain). In certain embodiments, such a hinge is a non-immunoglobulin hinge (i.e., a hinge not based on or derived from a wild type immunoglobulin hinge) and may be a stalk region of a type II C-lectin or CD molecule, an interdomain region that connect IgV-like or IgC-like domains of a cell surface receptor, or a derivative or functional variant thereof. Exemplary carboxyl terminal hinges, sometimes referred to as “back-end” hinges, includes those set forth in SEQ ID NOS: 384, 389-419, 593-596.

Other Components or Modifications

In certain embodiments, the immunoglobulin binding polypeptides of the invention may contain one or more additional domains or regions. Such additional regions may be a leader sequence (also referred to as “signal peptide”) at the amino-terminus for secretion of an expressed polypeptide. Exemplary leader peptides of this disclosure include natural leader sequences or others, such as those as set forth in SEQ ID NOS:193 and 13. In one embodiment, the polypeptides of the present invention make use of mature proteins that do not include the leader peptide (signal peptide). Accordingly, while certain sequences provided herein for binding domain proteins (such as for RON) include the leader peptide, the skilled person would readily understand how to determine the mature protein sequence from sequences including a signal peptide. In certain embodiments, it may be useful to include the leader sequence.

Additional regions may also be sequences at the carboxyl-terminus for identifying or purifying single chain polypeptides (e.g., epitope tags for detection or purification, such as a histidine tag, biotin, a FLAG® epitope, or any combination thereof).

Further optional regions may be additional amino acid residues (referred to as “junction amino acids” or “junction amino acid residues”) having a length of 1 to about 8 amino acids (e.g., about 2 to 5 amino acids), which may be resulted from use of specific expression systems or construct design for the polypeptides of the present disclosure. Such additional amino acid residues (for instance, about one, two, three, four or five additional amino acids) may be present at the amino or carboxyl terminus or between various regions or domains, such as between a binding domain and a heterodimerization domain, between a heterodimerization domain and a hinge, between a hinge and an Fc region portion, between domains of an Fc region portion (e.g., between CH2 and CH3 domains or between two CH3 domains), between a binding domain and a hinge, or between a variable domain and a linker. Exemplary junction amino acids amino-terminal to a hinge include RDQ (SEQ ID NO:607), RT, SS, SASS (SEQ ID NO:608) and SSS (SEQ ID NO:609). Exemplary junction amino acids carboxyl-terminal to a hinge include amino acids SG. Additional exemplary junction amino acids include SR.

The polypeptides of the present disclosure may also comprise linkers between the various domains as described herein. Exemplary linkers may include any of the linkers as provided in SEQ ID NOS:610-777. Illustrative linkers useful in linking the carboxyl terminus of a CH3 domain with an amino terminus of a CH1 or Cκ domain are provided in 798-805.

In certain embodiments, an immunoglobulin Fc region (e.g., CH2, CH3, and/or CH4 regions) may have an altered glycosylation pattern relative to an immunoglobulin reference sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831), such as N297 of the CH2 domain (EU numbering). Alternatively, the host cells producing the immunoglobulin binding polypeptides may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the Potelligent technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of fusion polypeptide of this disclosure. For example, a variety of glycosidase and/or mannosidase inhibitors provide one or more of desired effects of increasing ADCC activity, increasing Fc receptor binding, and altering glycosylation pattern. In certain embodiments, cells expressing fusion polypeptides of the instant disclosure are grown in a culture medium comprising a carbohydrate modifier at a concentration that increases the ADCC of immunoglycoprotein molecules produced by said host cell, wherein said carbohydrate modifier is at a concentration of less than 800 μM. In one embodiment, the cells expressing these polypeptides are grown in a culture medium comprising castanospermine or kifunensine, for instance, castanospermine at a concentration of 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in U.S. Pat. No. 7,846,434 or PCT Publication No. WO 2008/052030.

### Immunoglobulin Binding Polypeptide Structural Arrangements/Formats:

Immunoglobulin Binding Polypeptides: Antibodies

The present disclosure provides binding domain proteins in the form of antibodies or antigen binding fragments thereof, such as F(ab), F(ab′)₂, Fv, sFv, and scFv. Monoclonal antibodies specific for RON or other target of interest may be prepared, for example, using the techniques well known in the art, such as the techniques of Kohler and Milstein, Eur. J. Immunol. 6:511-519, 1976, and improvements thereto; Wayner E A, Hoffstrom B G. 2007. Methods Enzymol 426: 117-153; and Lane R D. 1985. J Immunol Methods 81: 223-228.

These methods include the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. One selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction.

Immunoglobulin Binding Polypeptides: SMIP/PIMS Molecules

In certain embodiments, a immunoglobulin binding polypeptide may comprise a “small modular immunopharmaceutical” (SMIP™). In this regard, the term SMIP™ refers to a highly modular compound class having enhanced drug properties over monoclonal and recombinant antibodies. SMIPs comprise a single polypeptide chain including a target-specific binding domain, based, for example, upon an antibody variable domain, in combination with a variable Fc region that permits the specific recruitment of a desired class of effector cells (such as, e.g., macrophages and natural killer (NK) cells) and/or recruitment of complement-mediated killing. Depending upon the choice of target and hinge regions, SMIPs can signal or block signaling via cell surface receptors. Thus, generally, SMIP proteins are binding domain-immunoglobulin fusion proteins that typically comprise from their amino termini to carboxyl termini: a binding domain derived from an immunoglobulin (e.g., a scFv), a hinge region, and an effector domain (e.g., IgG CH2 and CH3 regions). As used herein, “small modular immunopharmaceutical” or “SMIP™ products”, are as described in US Patent Publication Nos. 2003/133939, 2003/0118592, and 2005/0136049, and International Patent Publications WO02/056910, WO2005/037989, and WO2005/017148. Two identical SMIPs may form a homodimer with each other.

In some embodiments, a fusion protein of the invention comprising a RON binding domain may comprise a SMIP™ in reverse orientation, also referred to as a PIMS™ molecule such as those described in US Patent Publication No. 2009/0148447 and International Patent Publication WO2009/023386.

Immunoglobulin Binding Polypeptides: Scorpion/Xceptor Molecules

In certain embodiments the RON binding domains of the invention may be present within an immunoglobulin binding polypeptide such as those described in PCT application Nos. WO2007/146968 and US2009/059446. In this embodiment, the immunoglobulin binding polypeptides, also referred to as Scorpion/Xceptor polypeptides and multi-specific fusion proteins herein, may comprise a RON binding domain and a domain that binds a molecule other than RON (“heterologous binding domain”). In certain embodiments, the heterologous binding domain specifically binds to a target molecule including, but not limited to, Her1, Her2, Her3, CD3, epidermal growth factor receptor (EGFR), c-Met, histidine-rich glycoprotein (HRG), IGF-1, IGF-2, IGF-R1, IGF-R2, CD72, EGF, ERBB3, HGF, CD44, CD151, CEACAM6, TROP2, DR5, cKIT, CD27, IL6, CD40, VEGF-R, PDGF-R, TGFB, CD44v6, CD151, Wnt, and growth hormone-releasing hormone (GHRH).

It is contemplated that a RON binding domain may be at the amino-terminus and the heterologous binding domain at the carboxyl-terminus of a multi-specific fusion protein. It is also contemplated that a heterologous binding domain may be at the amino-terminus and the RON binding domain may be at the carboxyl-terminus. As set forth herein, the binding domains of this disclosure may be fused to each end of an intervening domain (e.g., an immunoglobulin constant region or sub-region thereof). Furthermore, the two or more binding domains may be each joined to an intervening domain via a linker, as described herein.

As used herein, an “intervening domain” refers to an amino acid sequence that simply functions as a scaffold for one or more binding domains so that the fusion protein will exist primarily (e.g., about 50% or more of a population of fusion proteins) or substantially (e.g., about 90% or more of a population of fusion proteins) as a single chain polypeptide in a composition. For example, certain intervening domains can have a structural function (e.g., spacing, flexibility, rigidity) or biological function (e.g., an increased half-life in plasma, such as in human blood). Exemplary intervening domains that can increase half-life of the fusion proteins of this disclosure in plasma include albumin, transferrin, a scaffold domain that binds a serum protein, or the like, or fragments thereof.

In certain embodiments, the intervening domain contained in a multi-specific fusion protein of this disclosure is a dimerization domain as described elsewhere herein. In certain embodiments, two identical multi-specific fusion proteins may form a homodimer with each other.

Exemplary structures of polypeptides comprising a RON binding domain, referred to herein as Xceptor molecules, include N-BD1-X-BD2-C, N-BD2-X-BD1-C, wherein N and C represent the amino-terminus and carboxyl-terminus, respectively; BD1 is a RON binding domain, such as an immunoglobulin-like or immunoglobulin variable region binding domain, or an ectodomain; X is an intervening domain, and BD2 is a binding domain that is a heterologous binding domain, i.e., a binding domain that binds a protein other than RON, such as, but not limited to, Her1, Her2, Her3, CD3, epidermal growth factor receptor (EGFR), c-Met, histidine-rich glycoprotein (HRG), IGF-1, IGF-2, IGF-R1, IGF-R2, CD72, EGF, ERBB3, HGF, CD44, CD151, CEACAM6, TROP2, DR5, cKIT, CD27, IL6, IL6-R, hyperIL6, CD40, VEGF-R, PDGF-R, TGFB, CD44v6, CD151, Wnt, and growth hormone-releasing hormone (GHRH). In certain embodiments, both BD1 and BD2 are immunoglobulin-like or immunoglobulin variable region binding domains, and the polypeptides may also be referred to as “Scorpion” proteins. In some constructs, X can comprise an immunoglobulin constant region or sub-region disposed between the first and second binding domains. In some embodiments, an immunoglobulin binding polypeptide has an intervening domain (X) comprising, from amino-terminus to carboxyl-terminus, a structure as follows: -L1-X-L2-, wherein L1 and L2 are each independently a linker comprising from about two to about 150 amino acids; and X is an immunoglobulin constant region or sub-region. In further embodiments, the immunoglobulin binding polypeptide will have an intervening domain that is albumin, transferrin, or another serum protein binding protein, wherein the fusion protein remains primarily or substantially as a single chain polypeptide in a composition.

In still further embodiments, an immunoglobulin binding polypeptide of this disclosure has the following structure: N-BD1-X-L2-BD2-C, wherein BD1 is a RON binding domain, such as a binding domain that is at least about 90% identical to a RON binding domain, such as those provided in SEQ ID NOS:87-93 and 157-159; —X— is -L1-CH2CH3-, wherein L1 is a first IgG1 hinge, optionally mutated by substituting the first or second cysteine and wherein —CH2CH3- is the CH2CH3 region of an IgG1 Fc domain; L2 is a linker selected from SEQ ID NOS:610-777; and BD2 is a heterologous binding domain that binds to a molecule other than RON.

In certain embodiments, the present disclosure provides a Scorpion/Xceptor that comprises multiple RON binding domains. In one embodiment, multiple RON binding domains may be linked in tandem and function as BD1 or BD2 as described in the structures herein above. In another embodiment, both binding domains of the Scorpion or Xceptor molecule may be RON binding domains (e.g., both BD1 and BD2 are RON binding domains.

Immunoglobulin Binding Polypeptides: Heterodimeric Molecules

The immunoglobulin binding polypeptides of the invention also include polypeptide heterodimers formed between two different single chain polypeptides via natural heterodimerization of an immunoglobulin CH1 region and an immunoglobulin light chain constant region (CL), such as those described further in the Examples herein and in U.S. provisional applications 61/290,840, 61/365,266, and 61/366,743; International application entitled “HETERODIMER BINDING PROTEINS AND USES THEREOF” in the name of inventors John W. Blankenship and Philip Tan, filed on Dec. 29, 2010; and International application entitled “POLYPEPTIDE HETERODIMERS AND USES THEREOF” in the name of inventors John W. Blankenship and Philip Tan, filed on Dec. 29, 2010.

A “polypeptide heterodimer,” “heterodimer,” or “Interceptor,” as used herein, refers to a dimer formed from two different single chain fusion polypeptides. In certain embodiments, a polypeptide heterodimer comprises at least one chain longer (long chain) than the other (short chain). This term does not include an antibody formed from four single chain polypeptides (i.e., two light chains and two heavy chains). A “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis).

A “single chain polypeptide” or a “single chain fusion polypeptide” is a single, linear and contiguous arrangement of covalently linked amino acids. It does not include two polypeptide chains that link together in a non-linear fashion, such as via an interchain disulfide bond (e.g., a half immunoglobulin molecule in which a light chain links with a heavy chain via a disulfide bond). In certain embodiments, a single chain polypeptide may have or form one or more intrachain disulfide bonds. A single chain polypeptide may or may not have a binding domain as described above. For example, in certain embodiments, two single chain polypeptides are constructed such that they form a heterodimer wherein one single chain polypeptide member of the heterodimer pair contains a binding domain and the other member of the pair does not. Thus, in this embodiment, the heterodimer formed functions as a binding molecule by function of the binding domain in one of the heterodimer member polypeptide chains.

An “immunoglobulin heterodimerization domain,” as used herein, refers to an immunoglobulin domain (“first immunoglobulin heterodimerization domain”) that preferentially interacts or associates with a different immunoglobulin domain (“second immunoglobulin heterodimerization domain”) wherein the interaction of the different heterodimerization domains substantially contributes to or efficiently promotes heterodimerization (i.e., the formation of a dimer between two different polypeptides, which is also referred to as a heterodimer). Representative immunoglobulin heterodimerization domains of the present disclosure include an immunoglobulin CH1 region, an immunoglobulin CL region (e.g., OK or CA isotypes), or derivatives thereof, as provided herein.

In certain embodiments, a polypeptide heterodimer as described herein comprises (i) a single chain polypeptide (“first single chain polypeptide”) having a first immunoglobulin heterodimerization domain and (ii) another single chain polypeptide (“second single chain polypeptide”) having a second heterodimerization domain that is not the same as the first heterodimerization domain, wherein the first and second heterodimerization domains substantially contribute to or efficiently promote formation of the polypeptide heterodimer. The interaction(s) between the first and second heterodimerization domains substantially contributes to or efficiently promotes the heterodimerization of the first and second single chain polypeptides if there is a statistically significant reduction in the dimerization between the first and second single chain polypeptides in the absence of the first heterodimerization domain and/or the second heterodimerization domain. In certain embodiments, when the first and second single chain polypeptides are co-expressed, at least about 60%, at least about 60% to about 70%, at least about 70% to about 80%, at least about 80% to about 90%, 91%, 92%, 93%, 94%, 95%, 96%, 97%, 98%, 99%, or 100%, and at least about 90% to about 92%, 93%, 94%, 95%, 96%, 97%, 98%, or 99% of the first and second single chain polypeptides form heterodimers with each other.

The heterodimerization technology described herein has one or more of the following advantages: (1) minimal immunogenicity of the polypeptide heterodimers because the dimers are formed via natural heterodimerization of an immunoglobulin CH1 region and an immunoglobulin CL region; (2) efficient production and purification of polypeptide heterodimers of the present disclosure is possible by co-expressing the two different single chain polypeptides, as shown in the examples; (3) the ability to mediate Fc effector functions (e.g., CDC, ADCC, ADCP), which can be modulated up or down by mutagenesis, and a longer serum half life because each chain of a polypeptide heterodimer according to the present disclosure has an Fc region portion (e.g., immunoglobulin CH2 and CH3 domains); and (4) polypeptide heterodimers of the present disclosure having a size that is typically smaller than an antibody molecule, which can allow for better tissue penetration, such as into a solid malignancy.

In one aspect, the present disclosure provides a heterodimer that comprises only a single binding domain, i.e., a RON binding domain. The heterodimer is comprised of a longer single chain polypeptide (which has a RON binding domain) and a shorter single chain polypeptide (which does not have any binding domain). In addition, both chains of the heterodimer further each comprise an Fc region portion (e.g., immunoglobulin CH2 and/or CH3 domains).

More particularly, the present disclosure provides single chain polypeptides and polypeptide heterodimers thereof that contain a single RON binding domain and have heterodimerization domain pairs of Cκ-CH1 or Cλ-CH1, or a combination of these pairs. In the simplest form, polypeptide heterodimers (also referred to as Interceptors) are made by co-expressing two unequal chains, one chain having a Cκ or Cλ domain and the other chain having a CH1 region. For example, the first chain polypeptide, designated the long chain, has a RON binding domain in the form of scFv and a CH1 heterodimerization domain, whereas the other chain, designated the short chain, lacks a binding domain but has a Cκ heterodimerization domain. Polypeptide heterodimers (Interceptors) will generally bind monovalently to the RON target protein and are ideal for blocking receptor/ligand or receptor/receptor interactions and preventing cell activation through receptor cross-linking. Other various advantages over, for example, a Fab, include a longer serum half-life and ease of purification due to the presence of the Fc domains. The interceptors may have a RON binding domain at the amino terminus or at the carboxyl terminus.

In another aspect, the present disclosure provides a polypeptide heterodimer (“multi-specific heterodimer”) formed by the association of two different single chain polypeptides wherein there is more than one binding domain, in particular at least one RON binding domain and at least one binding domain that binds a target other than RON. In certain embodiments, a heterodimer may be bispecific or may be multispecific. In this aspect, the present disclosure provides a polypeptide heterodimer wherein the first single chain polypeptide (SCP-I) comprises, consists essentially of, or consists of from one to four binding domains that specifically bind from one to four targets, a hinge (H-I), an immunoglobulin heterodimerization domain (HD-I), and an Fc region portion (FRP-I), whereas the second single chain polypeptide (SCP-II) comprises, consists essentially of, or consists of from zero to four binding domains that specifically bind from zero to four targets, a hinge (H-II), an immunoglobulin heterodimerization domain (HD-II), and an Fc region portion (FRP-II), provided that the polypeptide heterodimer comprises at least two binding domains that specifically bind to at least two different targets. The H-I and H-II may have the same sequence, but may be different. The FRP-I and FRP-II may have the same sequence, but may be different. The individual components of the polypeptide heterodimers of the present disclosure are described in detail herein.

If a single chain polypeptide of a multi-specific heterodimer comprises a single binding domain, the binding domain may be located either amino or carboxyl terminal to the Fc region portion of the single chain polypeptide. For example, a single chain polypeptide comprising two binding domains may have one binding domain located amino terminal and the other carboxyl terminal to the Fc region portion of the single chain polypeptide, or both binding domains may be amino terminal or both carboxyl terminal to the Fc region portion. In another example, a single chain polypeptide may comprise three binding domains wherein (a) two binding domains are amino terminal on different single chain proteins and the third binding domain is carboxyl terminal to the Fc region portion on either SCP-I or SCP-II, (b) two binding domains are carboxyl terminal on different single chain proteins and the third binding domain is amino terminal to the Fc region portion on either SCP-I or SCP-II. In still a further example, a polypeptide heterodimer may comprise four binding domains, wherein two binding domains are located amino terminal to the Fc region portion on different single chain proteins and the other two binding domains are located carboxyl terminal to the Fc region portion on different chains. Alternatively, in any of these embodiments, two binding domains may be linked to each other in tandem and located on either SCP-I or SCP-II or both, depending on the number of binding domains present—the tandem stacking is used when five to eight binding domains combined are present in SCP-I and SCP-II.

Thus, in certain embodiments, a heterodimer comprises at least one RON binding domain and may comprise one or more additional binding domains that bind to a heterologous target protein such as, but not limited to, TCR, CD3, Her1, Her2, Her3, epidermal growth factor receptor (EGFR), c-Met, histidine-rich glycoprotein (HRG), IGF-1, IGF-2, IGF-R1, IGF-R2, CD72, EGF, ERBB3, HGF, CD44, CD151, CEACAM6, TROP2, DR5, cKIT, CD27, IL6, IL6-R, hyperIL6, CD40, VEGF-R, PDGF-R, TGFB, CD44v6, CD151, Wnt, and growth hormone-releasing hormone (GHRH). In one particular embodiment, the first single chain polypeptide comprises an antiRON binding domain and the second single chain polypeptide comprises a TCR binding domain, such as a CD3 binding domain. In an additional embodiment, the first single chain polypeptide comprises an anti-RON binding domain and the second single chain polypeptide comprises an anti-c-Met binding domain. In a further embodiment, the first single chain polypeptide comprises an anti-RON binding domain and the second single chain polypeptide comprises an anti-CD19 binding domain.

Binding of a target by a binding domain modulates the interaction between the target (e.g., an antigen, a receptor, or a ligand) and another molecule. In certain embodiments, the binding of a target (e.g., a receptor) by a binding domain stimulates certain functions of the target (e.g., signal transduction) or brings different targets closer together for a biological effect (e.g., directing T cells to a tumor which in turn activates the T cells). In certain other embodiments, the binding of a target by a binding domain blocks the interaction between the target and another molecule and thus interferes, reduces or eliminates certain functions of the target.

In a related aspect, the present disclosure provides a polypeptide heterodimer formed by the association of two different single chain polypeptides that comprise two or more binding domains, each of which binds RON. Such a polypeptide heterodimer may be similar to a multi-specific heterodimer described herein except that its binding domains bind only to RON as opposed to the binding domains of the multi-specific heterodimer that bind at least two different targets.

### Making Immunoglobulin Binding Polypeptides

To efficiently produce any of the binding polypeptides described herein, a leader peptide may be used to facilitate secretion of expressed polypeptides. Using any of the conventional leader peptides (signal sequences) is expected to direct nascently expressed polypeptides into a secretory pathway and to result in cleavage of the leader peptide from the mature polypeptide at or near the junction between the leader peptide and the polypeptide. A particular leader peptide will be chosen based on considerations known in the art, such as using sequences encoded by polynucleotides that allow the easy inclusion of restriction endonuclease cleavage sites at the beginning or end of the coding sequence for the leader peptide to facilitate molecular engineering, provided that such introduced sequences specify amino acids that either do not interfere unacceptably with any desired processing of the leader peptide from the nascently expressed protein or do not interfere unacceptably with any desired function of a polypeptide if the leader peptide is not cleaved during maturation of the polypeptides. Exemplary leader peptides of this disclosure include natural leader sequences (i.e., those expressed with the native protein) or use of heterologous leader sequences, such as H₃N-MDFQVQIFSFLLISASVIMSRG(X)ₙ-CO₂H, wherein X is any amino acid and n is zero to three (SEQ ID NOS:778-781) or H₃N-MEAPAQLLFLLLLWLPDTTG-CO₂H (SEQ ID NO:782).

As noted herein, variants and derivatives of binding domains, such as ectodomains, light and heavy variable regions, and CDRs described herein, are contemplated. In one example, insertion variants are provided wherein one or more amino acid residues supplement a specific binding agent amino acid sequence. Insertions may be located at either or both termini of the protein, or may be positioned within internal regions of the specific binding agent amino acid sequence. Variant products of this disclosure also include mature specific binding agent products, i.e., specific binding agent products wherein a leader or signal sequence is removed, and the resulting protein having additional amino terminal residues. The additional amino terminal residues may be derived from another protein, or may include one or more residues that are not identifiable as being derived from a specific protein. Polypeptides with an additional methionine residue at position −1 are contemplated, as are polypeptides of this disclosure with additional methionine and lysine residues at positions −2 and −1. Variants having additional Met, Met-Lys, or Lys residues (or one or more basic residues in general) are particularly useful for enhanced recombinant protein production in bacterial host cells.

As used herein, “amino acids” refer to a natural (those occurring in nature) amino acid, a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (His or H), and derivatives thereof; and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (Gly or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration.

Substitution variants include those polypeptides wherein one or more amino acid residues in an amino acid sequence are removed and replaced with alternative residues. In some embodiments, the substitutions are conservative in nature; however, this disclosure embraces substitutions that are also non-conservative. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 1 (see WO 97/09433, page 10, published Mar. 13, 1997), immediately below.

TABLE 1
Conservative Substitutions I

| Side Chain | Characteristic | Amino Acid |
|---|---|---|
| Aliphatic | Non-polar | G, A, P, I, L, V |
| | Polar - uncharged | S, T, M, N, Q |
| | Polar - charged | D, E, K, R |
| Aromatic | | H, F, W, Y |
| Other | | N, Q, D, E |

Alternatively, conservative amino acids can be grouped as described in Lehninger (Biochemistry, Second Edition; Worth Publishers, Inc. NY:N.Y. (1975), pp. 71-77) as set out in Table 2, immediately below.

TABLE 2
Conservative Substitutions II

| Side Chain | Characteristic | Amino Acid |
|---|---|---|
| Non-polar (hydrophobic) | Aliphatic: | A, L, I, V, P |
| | Aromatic | F, W |
| | Sulfur-containing | M |
| | Borderline | G |
| Uncharged-polar | Hydroxyl | S, T, Y |
| | Amides | N, Q |
| | Sulfhydryl | C |
| | Borderline | G |
| Positively Charged (Basic) | | K, R, H |
| Negatively Charged | | D, E |
| (Acidic) | | |

Variants or derivatives can also have additional amino acid residues which arise from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants which result from expression in other vector systems are also contemplated, including those wherein histidine tags are incorporated into the amino acid sequence, generally at the carboxyl and/or amino terminus of the sequence.

Deletion variants are also contemplated wherein one or more amino acid residues in a binding domain of this disclosure are removed. Deletions can be effected at one or both termini of the fusion protein, or from removal of one or more residues within the amino acid sequence.

In certain illustrative embodiments, immunoglobulin binding polypeptides of the invention are glycosylated, the pattern of glycosylation being dependent upon a variety of factors including the host cell in which the protein is expressed (if prepared in recombinant host cells) and the culture conditions.

This disclosure also provides derivatives of immunoglobulin binding polypeptides. In certain embodiments, the modifications are covalent in nature, and include for example, chemical bonding with polymers, lipids, other organic, and inorganic moieties. Derivatives of this disclosure may be prepared to increase circulating half-life of a specific binding domain polypeptide, or may be designed to improve targeting capacity for the polypeptide to desired cells, tissues, or organs.

This disclosure further embraces binding polypeptides that are covalently modified or derivatized to include one or more water-soluble polymer attachments such as polyethylene glycol, polyoxyethylene glycol, or polypropylene glycol, as described U.S. Pat. Nos. 4,640,835; 4,496,689; 4,301,144; 4,670,417; 4,791,192 and 4,179,337. Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)-derivatized proteins. Water-soluble polymers may be bonded at specific positions, for example at the amino terminus of the proteins and polypeptides according to this disclosure, or randomly attached to one or more side chains of the polypeptide. The use of PEG for improving therapeutic capacities is described in U.S. Pat. No. 6,133,426.

In one embodiment, the immunoglobulin binding polypeptide is a fusion protein that comprises an immunoglobulin or an Fc fusion protein. Such a fusion protein can have a long half-life, e.g., several hours, a day or more, or even a week or more, especially if the Fc domain is capable of interacting with FcRn, the neonatal Fc receptor. The binding site for FcRn in an Fc domain is also the site at which the bacterial proteins A and G bind. The tight binding between these proteins can be used as a means to purify antibodies or fusion proteins of this disclosure by, for example, employing protein A or protein G affinity chromatography during protein purification. In certain embodiments, the Fc domain of the fusion protein is optionally mutated to eliminate interaction with FcγRI-III while retaining FcRn interaction.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure polypeptide are ion-exchange chromatography; exclusion chromatography; polyacrylamide gel electrophoresis; and isoelectric focusing. Particularly efficient methods of purifying peptides are fast protein liquid chromatography and HPLC.

Certain aspects of the present disclosure concern the purification, and in particular embodiments, the substantial purification, of a polypeptide. The terms “purified polypeptide” and “purified fusion protein” are used interchangeably herein and refer to a composition, isolatable from other components and that is purified to any degree relative to its naturally obtainable state. A purified polypeptide therefore also refers to a polypeptide, free from the environment in which it may naturally occur.

Generally, “purified” will refer to a polypeptide composition that has been subjected to fractionation to remove various other components, and which composition substantially retains its expressed biological activity. Where the term “substantially purified” is used, this designation refers to a polypeptide composition in which the polypeptide forms the major component of the composition, such as constituting about 50%, about 60%, about 70%, about 80%, about 90%, about 95%, about 99% or more of the polypeptide, by weight, in the composition.

Various methods for quantifying the degree of purification are known to those of skill in the art in light of the present disclosure. These include, for example, determining the specific binding activity of an active fraction, or assessing the amount of polypeptide in a fraction by SDS/PAGE analysis. A preferred method for assessing the purity of a protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed fusion protein exhibits a detectable binding activity.

Various techniques suitable for use in protein purification are well known to those of skill in the art. These include, for example, precipitation with ammonium sulfate, PEG, antibodies and the like, or by heat denaturation, followed by centrifugation; chromatography steps such as ion exchange, gel filtration, reverse phase, hydroxylapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein.

There is no general requirement that the binding polypeptide always be provided in its most purified state. Indeed, it is contemplated that less substantially purified proteins will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in greater purification than the same technique utilizing a low pressure chromatography system. Methods exhibiting a lower degree of relative purification may have advantages in total recovery of protein product, or in maintaining binding activity of an expressed protein.

It is known that the migration of a polypeptide can vary, sometimes significantly, with different conditions of SDS/PAGE (Capaldi et al. (1977) Biochem. Biophys. Res. Comm. 76:425). It will therefore be appreciated that under differing electrophoresis conditions, the apparent molecular weights of purified or partially purified fusion protein expression products may vary.

### Polynucleotides, Expression Vectors, and Host Cells

This disclosure provides polynucleotides (isolated or purified or pure polynucleotides) encoding the immunoglobulin binding polypeptides as described herein, vectors (including cloning vectors and expression vectors) comprising such polynucleotides, and cells (e.g., host cells) transformed or transfected with a polynucleotide or vector according to this disclosure.

In certain embodiments, a polynucleotide (DNA or RNA) encoding a binding domain of this disclosure, or polypeptides containing one or more such binding domains is contemplated. Expression cassettes encoding fusion protein constructs are provided in the examples and the sequence listing appended hereto.

The present disclosure also relates to vectors that include a polynucleotide of this disclosure and, in particular, to recombinant expression constructs. In one embodiment, this disclosure contemplates a vector comprising a polynucleotide encoding a RON binding domain or other binding domain and polypeptides thereof, along with other polynucleotide sequences that cause or facilitate transcription, translation, and processing of such protein-encoding sequences.

Appropriate cloning and expression vectors for use with prokaryotic and eukaryotic hosts are described, for example, in Sambrook et al., Molecular Cloning: A Laboratory Manual, Second Edition, Cold Spring Harbor, N.Y., (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein.

As used herein, “vector” means a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences.

In certain embodiments, expression constructs are derived from plasmid vectors. Illustrative constructs include modified pNASS vector (Clontech, Palo Alto, Calif.), which has nucleic acid sequences encoding an ampicillin resistance gene, a polyadenylation signal and a T7 promoter site; pDEF38 and pNEF38 (CMC ICOS Biologics, Inc.), which have a CHEF1 promoter; and pD18 (Lonza), which has a CMV promoter. Other suitable mammalian expression vectors are well known (see, e.g., Ausubel et al., 1995; Sambrook et al., supra; see also, e.g., catalogs from Invitrogen, San Diego, Calif.; Novagen, Madison, Wis.; Pharmacia, Piscataway, N.J.). Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, the protein-encoding nucleic acids as provided herein may be included in any one of a variety of expression vector constructs as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (Current Protocols in Molecular Biology, Greene Publ. Assoc. Inc. & John Wiley & Sons, Inc., Boston, Mass., 1993); Sambrook et al. (Molecular Cloning, Second Ed., Cold Spring Harbor Laboratory, Plainview, N.Y., 1989); Maniatis et al. (Molecular Cloning, Cold Spring Harbor Laboratory, Plainview, N.Y., 1982); Glover (Ed.) (DNA Cloning Vol. I and II, IRL Press, Oxford, UK, 1985); Hames and Higgins (Eds.) (Nucleic Acid Hybridization, IRL Press, Oxford, UK, 1985); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least about 80%, 85%, 90%, 91%, 92%, 93%, 94%, 95%, 96%, 97%, 98%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y., 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

A further aspect of this disclosure provides a host cell transformed or transfected with, or otherwise containing, any of the polynucleotides or vector/expression constructs of this disclosure. The polynucleotides or cloning/expression constructs of this disclosure are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of this disclosure when harboring a polynucleotide, vector, or protein according to this disclosure include, in addition to a subject's own cells (e.g., a human patient's own cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. The selection of an appropriate host is within the scope of those skilled in the art from the teachings herein. Host cells that glycosylate the fusion proteins of this disclosure are contemplated.

The term “recombinant host cell” (or simply “host cell”) refers to a cell containing a recombinant expression vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Recombinant host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman (1981) Cell 23:175, and other cell lines capable of expressing a compatible vector, for example, the C127, 3T3, CHO, HeLa and BHK cell lines. Mammalian expression vectors will comprise an origin of replication, a suitable promoter and, optionally, enhancer, and also any necessary ribosome binding sites, polyadenylation site, splice donor and acceptor sites, transcriptional termination sequences, and 5′-flanking nontranscribed sequences, for example, as described herein regarding the preparation of multivalent binding protein expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including calcium phosphate transfection, DEAE-Dextran-mediated transfection, or electroporation (Davis et al. (1986) Basic Methods in Molecular Biology).

In one embodiment, a host cell is transduced by a recombinant viral construct directing the expression of a protein or polypeptide according to this disclosure. The transduced host cell produces viral particles containing expressed protein or polypeptide derived from portions of a host cell membrane incorporated by the viral particles during viral budding.

### Compositions and Method for Using Immunoglobulin Binding Polypeptides

The present disclosure further provides for compositions comprising any of the immunoglobulin binding polypeptides as described herein. The immunoglobulin binding polypeptides of the invention are RON binding polypeptides. The terms “immunoglobulin binding polypeptide,” “binding polypeptide,” “RON binding polypeptide,” “fusion protein,” and “fusion polypeptide” are used interchangeably herein unless specified to the contrary.

The present disclosure also provides pharmaceutical compositions and unit dose forms that comprise any format of the immunoglobulin binding polypeptides (e.g., anti-RON antibody, SMIP™, PIMS, Xceptor™, homodimeric and heterodimeric Interceptor) as well as methods for using the compositions comprising any format of the RON binding polypeptides described herein.

Compositions of immunoglobulin binding polypeptides of this disclosure generally comprise a binding polypeptide of any format described herein (e.g., anti-RON antibody, SMIP™, PIMS™, Xceptor™, homodimeric and heterodimeric Interceptor) in combination with a pharmaceutically acceptable excipient, including pharmaceutically acceptable carriers and diluents. Pharmaceutical acceptable excipients will be nontoxic to recipients at the dosages and concentrations employed. They are well known in the pharmaceutical art and described, for example, in Rowe et al., Handbook of Pharmaceutical Excipients: A Comprehensive Guide to Uses, Properties, and Safety, 5^{th} Ed., 2006.

Pharmaceutically acceptable carriers for therapeutic use are also well known in the pharmaceutical art, and are described, for example, in Remington's Pharmaceutical Sciences, Mack Publishing Co. (A. R. Gennaro (Ed.) 1985). Exemplary pharmaceutically acceptable carriers include sterile saline and phosphate buffered saline at physiological pH. Preservatives, stabilizers, dyes and the like may be provided in the pharmaceutical composition. In addition, antioxidants and suspending agents may also be used.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose, dextrins), chelating agents (e.g., EDTA), glutathione and other stabilizers and excipients. Neutral buffered saline or saline mixed with nonspecific serum albumin are exemplary diluents. In one embodiment, the product is formulated as a lyophilizate using appropriate excipient solutions (e.g., sucrose) as diluents.

The present disclosure also provides a method for treating a disease or disorder associated with, for example, excessive receptor-mediated signal transduction, comprising administering to a patient in need thereof a therapeutically effective amount of any of the RON binding proteins described herein.

Exemplary diseases or disorders associated with excess receptor-mediated signal transduction include cancer (e.g., solid malignancy and hematologic malignancy) and a variety of inflammatory disorders.

In one embodiment, the present disclosure provides a method for treating, reducing the severity of or preventing inflammation or an inflammatory disease (see e.g., Camp et al. Ann. Surg. Oncol. 12:273-281 (2005); Correll, P. H. et al., Genes Funct. 1997 February; 1(1):69-83). For example, one embodiment of the invention provides a method for the treatment of inflammation or an inflammatory disease including, but not limited to, Crohn's disease, colitis, dermatitis, psoriasis, diverticulitis, hepatitis, irritable bowel syndrom (IBS), rheumatoid arthritis, asthma, lupus erythematous, nephritis, Parkinson's disease, ulcerative colitis, multiple sclerosis (MS), Alzheimer's disease, arthritis, and various cardiovascular diseases such as atherosclerosis and vasculitis. In certain embodiments, the inflammatory disease is selected from the group consisting of rheumatoid arthritis, diabetes, gout, cryopyrin-associated periodic syndrome, and chronic obstructive pulmonary disorder comprising administering a therapeutically effective amount of the immunoglobulin binding polypeptide of the invention or composition of the invention to a patient. In this regard, one embodiment provides a method of treating, reducing the severity of or preventing inflammation or an inflammatory disease by administering to a patient in need thereof a therapeutically effective amount of a RON binding protein as disclosed herein.

Some studies have implicated RON in innate immunity and TNF-alpha related pathologies (Nikolaidis et al., 2010, Nov. 18, Innate Immun. (epub ahead of print); Wilson et al., 2008, J. Immunol. 181:2303). Further studies indicate that RON inhibits HIV-1 transcriptions in monocytes/macrophages (Lee et al., 2004, J. Immunol. 173:6864). Accordingly, in certain embodiments, the RON binding proteins of the present disclosure may be used in the treatment of sepsis, periotonitis, ulcerative colitis, AIDS, rheumatoid arthritis, and other TNF-alpha related pathologies.

In one aspect, the present disclosure provides a method for inhibiting growth, metastasis or metastatic growth of a malignancy (e.g., a solid malignancy or a hematologic malignancy), comprising administering to a patient in need thereof an effective amount RON binding polypeptide of any format described herein or a composition thereof.

A wide variety of cancers, including solid malignancy and hematologic malignancy, are amenable to the compositions and methods disclosed herein. Types of cancer that may be treated include, but are not limited to: adenocarcinoma of the breast, prostate, pancreas, colon and rectum; all forms of bronchogenic carcinoma of the lung (including squamous cell carcinoma, adenocarcinoma, small cell lung cancer and non-small cell lung cancer); myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; and glioblastoma multiforme. The types of cancers that may be treated also include, but are not limited to, angiokeratoma; angiolymphoid hyperplasia with eosinophilia; angioma sclerosing; angiomatosis; glomangioma; hemangioendothelioma; hemang ioma; hemang iopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Additional exemplary cancers that are also amenable to the compositions and methods disclosed herein are B-cell cancers, including B-cell lymphomas [such as various forms of Hodgkin's disease, non-Hodgkins lymphoma (NHL) or central nervous system lymphomas], leukemias [such as acute lymphoblastic leukemia (ALL), chronic lymphocytic leukemia (CLL), Hairy cell leukemia and chronic myoblastic leukemia] and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

Any format of the immunoglobulin binding polypeptides or compositions thereof of the present disclosure may be administered orally, topically, transdermally, parenterally, by inhalation spray, vaginally, rectally, or by intracranial injection, or any combination thereof. In one embodiment, the RON binding proteins or compositions thereof are administered parenterally. The term “parenteral,” as used herein, includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and/or surgical implantation at a particular site is contemplated as well. For instance, the invention includes methods of treating a patient comprising administering a therapeutically effective amount of the immunoglobulin binding polypeptide of the invention or composition of the invention to a patient by intravenous injection.

The therapeutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.01 mg/kg and 1000 mg/kg (e.g., about 0.1 to 1 mg/kg, about 1 to 10 mg/kg, about 10-50 mg/kg, about 50-100 mg/kg, about 100-500 mg/kg, or about 500-1000 mg/kg) body weight (which can be administered as a single dose, daily, weekly, monthly, or at any appropriate interval) of active ingredient may be administered depending on the potency of an immunoglobulin binding polypeptide of this disclosure.

Also contemplated is the administration of immunoglobulin binding polypeptides or compositions thereof in combination with a second agent. A second agent may be one accepted in the art as a standard treatment for a particular disease state or disorder, such as in cancer or in an inflammatory disorder. Exemplary second agents contemplated include polyclonal antibodies, monoclonal antibodies, immunoglobulin-derived fusion proteins, chemotherapeutics, ionizing radiation, steroids, NSAIDs, anti-infective agents, or other active and ancillary agents, or any combination thereof.

A variety of other therapeutic agents may find use for administration with the immunoglobulin binding polypeptides described herein. In one embodiment, the immunoglobulin binding polypeptide is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, immune selective anti-inlammatory derivatives (imSAIDS), methotrexate, sulfasalazine, leflunomide, anti-TNF medications, cyclophosphamide and mycophenolate.

Second agents useful in combination with the immunoglobulin binding protein or compositions thereof provided herein include anti-infective drugs, such as antibiotics antiviral and antifungal agents. Exemplary antibiotics include, for example, penicillin, cephalosporins, aminoglycosides, macrolides, quinolones and tetracyclines. Exemplary antiviral agents include, for example, reverse transcriptase inhibitors, protease inhibitors, antibodies, and interferons. Exemplary antifungal agents include, for example, polyene antifungals (e.g., natamycin and rimocidin), imidazole, triazole, or thiazole antifungals (e.g., miconazone, ketoconazole, fluconazole, itraconazole, and abaungin), allylamines (e.g., terbinafine, naftifine), and echinocandins (e.g., anidulafungin and casposungin).

In certain embodiments, an immunoglobulin binding polypeptide and a second agent act synergistically. In other words, these two compounds interact such that the combined effect of the compounds is greater than the sum of the individual effects of each compound when administered alone (see, e.g., Berenbaum, Pharmacol. Rev. 41:93, 1989).

In certain other embodiments, an immunoglobulin binding polypeptide and a second agent act additively. In other words, these two compounds interact such that the combined effect of the compounds is the same as the sum of the individual effects of each compound when administered alone.

Second agents useful in combination with immunoglobulin binding proteins or compositions thereof provided herein may be steroids, NSAIDs, mTOR inhibitors (e.g., rapamycin (sirolimus), temsirolimus, deforolimus, everolimus, zotarolimus, curcumin, farnesylthiosalicylic acid), calcineurin inhibitors (e.g., cyclosporine, tacrolimus), anti-metabolites (e.g., mycophenolic acid, mycophenolate mofetil), polyclonal antibodies (e.g., anti-thymocyte globulin), monoclonal antibodies (e.g., daclizumab, basiliximab, HERCEPTIN® (trastuzumab), ERBITUX® (Cetuximab)), and CTLA4-Ig fusion proteins (e.g., abatacept or belatacept).

Second agents useful for inhibiting growth of a solid malignancy, inhibiting metastasis or metastatic growth of a solid malignancy, or treating or ameliorating a hematologic malignancy include chemotherapeutic agents, ionizing radiation, and other anti-cancer drugs. Examples of chemotherapeutic agents contemplated as further therapeutic agents include alkylating agents, such as nitrogen mustards (e.g., mechlorethamine, cyclophosphamide, ifosfamide, melphalan, and chlorambucil); bifunctional chemotherapeutics (e.g., bendamustine); nitrosoureas (e.g., carmustine (BCNU), lomustine (CCNU), and semustine (methyl-CCNU)); proteasome inhibitors (e.g. VELCADE® (bortezomib)); tyrosine kinase inhibitors (e.g. TARCEVA® (erlotinib) and TYKERB® (lapatinib)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogues (e.g., methotrexate, trimetrexate, and pemetrexed (multi-targeted antifolate)); pyrimidine analogues (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogues (e.g, 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (MIH) and procarbazine.

In certain embodiments, second agents useful for inhibiting growth metastasis or metastatic growth of a malignancy include multi-specific binding polypeptides or binding polypeptide heterodimers according to the present disclosure that bind to cancer cell targets other than RON. In certain other embodiments, second agents useful for such treatments include polyclonal antibodies, monoclonal antibodies, and immunoglobulin-derived fusion proteins that bind to cancer cell targets.

Further therapeutic agents contemplated by this disclosure are referred to as immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases aside from rheumatoid arthritis.

Exemplary NSAIDs are chosen from the group consisting of ibuprofen, naproxen, naproxen sodium, Cox-2 inhibitors such as VIOXX® (rofecoxib) and CELEBREX® (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

It is contemplated the binding molecule composition and the second active agent may be given simultaneously in the same formulation. Alternatively, the second agents may be administered in a separate formulation but concurrently (i.e., given within less than one hour of each other).

In certain embodiments, the second active agent may be administered prior to administration of a RON binding polypeptide or a composition thereof. Prior administration refers to administration of the second active agent at least one hour prior to treatment with the RON binding protein or the composition thereof. It is further contemplated that the active agent may be administered subsequent to administration of the binding molecule composition. Subsequent administration is meant to describe administration at least one hour after the administration of the binding molecule or the composition thereof.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

As an additional aspect, the disclosure includes kits which comprise one or more compounds or compositions useful in the methods of this disclosure packaged in a manner which facilitates their use to practice methods of the disclosure. In a simplest embodiment, such a kit includes a compound or composition described herein as useful for practice of a method of the disclosure packaged in a container such as a sealed bottle or vessel, with a label affixed to the container or included in the package that describes use of the compound or composition to practice the method of the disclosure. Preferably, the compound or composition is packaged in a unit dosage form. The kit may further include a device suitable for administering the composition according to a preferred route of administration or for practicing a screening assay. The kit may include a label that describes use of the binding molecule composition(s) in a method of the disclosure.

### EXAMPLES

### Example 1

### Generation of RON Binding Molecules

Anti-RON antibodies were generated and various recombinant molecules containing anti-RON binding domains from these antibodies were constructed as described below.

RON-expressing cell lines were generated using full length RON/MST1R was obtained from OriGene Technologies (#SC309913, Rockville, Md.; GENBANK™ Accession Number NM_{—}002447 gi:153946392; SEQ ID NO:783, encoding the amino acid sequence provided in SEQ ID NO:784. Full length Macaca mulatta RON was synthesized by Blue Heron Biotechnology (Bothell, Wash.) based on Ensembl sequence ENSMMUT00000004738. Both human and macaque RON open reading frame sequences were subcloned into pcDNA™3.1/Hygro (+) (Invitrogen, Carlsbad, Calif.). NIH/3T3 cells (ATCC, Manassas, Va.) were transfected with Bcg I- (human) or Bgl II- (macaque) linearized full length RON in pcDNA™3.1/Hygro (+) using the polyethylenimine technique (Boussif et al. 1995, Proc. Natl. Acad. Sci. USA 92:7297-7301). From the transiently transfected pools, stable cell lines over-expressing human or macaque full length RON were cloned. As RON-negative cell line controls, NIH/3T3 cells were transfected with supercoiled pcDNA™3.1/Hygro/lacZ (Invitrogen) or pcDNA™3.1/Hygro (+) and cloned to generate stable cell lines as described above.

Novel antibodies against RON were generated using previously established protocols (Wayner and Hoffstrom 2007) and the RON-expressing cell lines described above as immunogen. For RON-e01 antibodies, following cell line boosts, mice received a boost of 50 μg recombinant RON Sema-PSI protein (R&D Systems #1947-MS, Minneapolis, Minn.). This protein includes the Sema and PSI domains of human RON (Glu 25-Leu 571) coupled to a carboxyl-terminal histidine tag and expressed in the NS0 mouse myeloma cell line. For RON-f01 antibodies, following the cell line boosts, the mouse received a boost of 20 μg recombinant RON protein. One additional boost and the pre-fusion boost were performed with 50 μl packed NIH/3T3 cells stably expressing macaque RON.

Hybridomas were generated by fusion of the B cells from the spleens of immunized animals with a clone of the mouse myeloma cell line P3-X63-Ag8.653 (Kearney et al. 1979) (designated P3-X63-Ag8.653.3.12.11) using standard methods (Lane 1985).

Hybridoma culture supernatants were screened for the ability to inhibit RON phosphorylation induced by macrophage stimulating protein (MSP, R&D Systems, Minneapolis, Minn.) in MDA-MB-453 cells. MDA-MB-453 cells were plated overnight at 5×10⁴ cells/well in a 96-well tissue culture coated microplate in DMEM+10% FBS. The following day, the media was aspirated and either replaced with serum-free DMEM for a 3-hour serum starvation 37° C. prior to incubation with hybridoma supernatant or replaced directly with hybridoma supernatant for a 1-hour blocking step at 37° C. Blocking treatments were aspirated and cells were stimulated for 10 min. at room temperature with 3 nM MSP in serum-free DMEM containing 100 μM Na₃VO₄. Immediately after MSP stimulation, cells were lysed on ice in 1× Sample Diluent Concentrate 2 (R&D Systems, Minneapolis, Minn.) supplemented with HALT™ Protease Inhibitor Cocktail (Thermo Fisher Scientific, Rockford, Ill.), HALT™ Phosphatase Inhibitor Cocktail (Thermo Fisher), and 1 mM Na₃VO₄. Cell lysates were analyzed on the DuoSet IC Human phospho-MSP R/Ron ELISA (R&D Systems, Minneapolis, Minn.).

Subsequently, supernatants from hybridoma pools identified in the RON phosphorylation assay were examined for the presence of anti-RON antibodies by flow cytometry on RON-negative NIH/3T3 cells versus NIH/3T3 cells over-expressing human or macaque RON.

Hybridomas of interest from pools passing both screens were weaned from HAT selection into hypoxanthine-thymidine (HT) and were cloned by limiting dilution in the presence of BM Condimed H1 (Roche Applied Science, Indianapolis, Ind.). Clones were re-tested for both binding and functional activity. RON-e01 (11H09 hybridoma) and RON-f01 (4C04 hybridoma) were selected at this stage for further testing. The VL and VH regions of both antibodies were identified by 5′-RACE (Rapid Amplification of cDNA Ends) and converted into SMIP and Interceptor formats.

Binding domains specific for RON include a 11H09 scFv as set forth in SEQ ID NOS:43 (polynucleotide) and 87 (amino acid) and a 4C04 scFv as set forth in SEQ ID NO: 127 (polynucleotide) and 157 (amino acid). Humanized versions of the 4C04 scFv RON binding domains are set forth in SEQ ID NOS: 128-129 (polynucleotide) and 158-159 (amino acid) and the humanized version of the 11H09 scFv RON binding domains are set forth in SEQ ID NOS: 44-49 (polynucleotide) and 88-93 (amino acid).

The light chain amino acid sequence of the 4C04 scFv is set forth in SEQ ID NO:152, and its CDR1, CDR2, and CDR3 are set forth in SEQ ID NOS:141-143, respectively. The heavy chain amino acid sequence of the 4C04 scFv is set forth in SEQ ID NO:153, and its CDR1, CDR2, and CDR3 are set forth in SEQ ID NOS:144-146, respectively. A variant of the heavy chain amino acid sequence of the 4C04 scFv is set forth in SEQ ID NO:176 where the terminal leucine has been changed to a serine residue. This variant heavy chain sequence is used in numerous of the binding domain constructs described herein, such as those disclosed in SEQ ID NOS:160-175.

The light chain amino acid sequence of the 11H09 scFv is set forth in SEQ ID NO:80, and its CDR1, CDR2, and CDR3 are set forth in SEQ ID NOS:67-69, respectively. The heavy chain amino acid sequence of the 11H09 scFv is set forth in SEQ ID NO:81, and its CDR1, CDR2, and CDR3 are set forth in SEQ ID NOS:70-72, respectively.

SMIP molecules comprising 4C04-derived RON binding domains are provided in SEQ ID NOS:130-132 (polynucleotides) and 160-168 (amino acid). SEQ ID NOS:160, 163, and 166 include the 20 amino acid Vk3 leader sequence; SEQ ID NOS:161, 162, 164, 165, 167 and 168 do not include a leader sequence; SEQ ID NOS: 162, 165, and 168 have the terminal lysine residue removed. SEQ ID NOS:131, 132 and 163-168 are humanized. The Vk3 leader sequence is set forth in SEQ ID NO:13, encoded by the polynucleotide sequence of SEQ ID NO:1.

SMIP molecules comprising 11H09-derived RON binding domains are provided in SEQ ID NOS:50-56 (polynucleotides) and 94-114. SEQ ID NOS:94, 97, 100, 103, 106, 109, and 112 contain the 20 amino acid Vk3 leader sequence of SEQ ID NO:13; SEQ ID NOS:95-96, 98-99, 101-102, 104-105, 107-108, 110-111 and 113-114 do not contain a leader sequence; SEQ ID NOS:96, 99, 102, 105, 108, 111, 114 have the terminal lysine residue removed. SEQ ID NOS:99-114 are humanized.

Table 3 below summarizes the 4C04 and 11H09 RON binding antibody and SMIP molecules generated and lists the corresponding SEQ ID NOs.

TABLE 3
Summary of 4C04 and 11H09 RON Binding Molecules

| Patent | | | Amino |
|---|---|---|---|
| Protein | | Polynucleotide | Acid SEQ |
| Name | Format Description | SEQ ID NOs | ID NOs |
| RON-e01 | 11H09 murine VL | 36 | 80 |
| | 11H09 murine VL CDR1 | | 67 |
| | 11H09 murine VL CDR2 | | 68 |
| | 11H09 murine VL CDR3 | | 69 |
| RON-e01 | 11H09 murine VH | 37 | 81 |
| | 11H09 murine VH CDR1 | | 70 |
| | 11H09 murine VH CDR2 | | 71 |
| | 11H09 murine VH CDR3 | | 72 |
| RON-e01h6 | Humanized 11H09 murine | 38 | 82 |
| | VL using K02098 human | | |
| | germline framework | | |
| RON-e01h7 | Humanized 11H09 murine | 39 | 83 |
| | VL using Y14865 human | | |
| | germline framework | | |
| RON-e01h8 | Humanized 11H09 murine | 40 | 84 |
| | VH using X62106 human | | |
| | germline framework | | |
| RON-e01h9 | Humanized 11H09 murine | 41 | 85 |
| | VH using M99637 human | | |
| | germline framework | | |
| RON-e01h0 | Humanized 11H09 murine | 42 | 86 |
| | VH using X92343 human | | |
| | germline framework | | |
| RON-e02 | VLVH ScFv | 43 | 87 |
| RON-e07h68 | Humanized VLVH scFV | 44 | 88 |
| RON-e08h78 | Humanized VLVH scFV | 45 | 89 |
| RON-e09h69 | Humanized VLVH scFV | 46 | 90 |
| RON-e10h79 | Humanized VLVH scFV | 47 | 91 |
| RON-e11h60 | Humanized VLVH scFV | 48 | 92 |
| RON-e12h70 | Humanized VLVH scFV | 49 | 93 |
| RON-e02 | 11H09 murine SMIP | 50 | 94 (w/ |
| | | | leader), 95 |
| | | | (w/out |
| | | | leader), 96 |
| | | | (w/out |
| | | | leader, no |
| | | | terminal |
| | | | lysine) |
| RON-e07h68 | Humanized 11H09 SMIP: | 51 | 97 (w/ |
| | e01h6 VL/e01h8 VH | | leader), 98 |
| | | | (w/out |
| | | | leader), 99 |
| | | | (w/out |
| | | | leader, no |
| | | | terminal |
| | | | lysine) |
| RON-e08h78 | Humanized 11H09 SMIP: | 52 | 100 (w/ |
| | e01h7 VL/e01h8 VH | | leader), |
| | | | 101 (w/out |
| | | | leader), |
| | | | 102 (w/out |
| | | | leader, no |
| | | | terminal |
| | | | lysine) |
| RON-e09h69 | Humanized 11H09 SMIP: | 53 | 103 (w/ |
| | e01h6 VL/e01h9 VH | | leader), |
| | | | 104 (w/out |
| | | | leader), |
| | | | 105 (w/out |
| | | | leader, no |
| | | | terminal |
| | | | lysine) |
| RON-e10h79 | Humanized 11H09 SMIP: | 54 | 106 (w/ |
| | e01h7 VL/e01h9 VH | | leader), |
| | | | 107 (w/out |
| | | | leader), |
| | | | 108 (w/out |
| | | | leader, no |
| | | | terminal |
| | | | lysine) |
| RON-e11h60 | Humanized 11H09 SMIP: | 55 | 109 (w/ |
| | e01h6 VL/e01h0 VH | | leader), |
| | | | 110 (w/out |
| | | | leader), |
| | | | 111 (w/out |
| | | | leader, no |
| | | | terminal |
| | | | lysine) |
| RON-e12h70 | Humanized 11H09 SMIP: | 56 | 112 (w/ |
| | e01h7 VL/e01h0 VH | | leader), |
| | | | 113 (w/out |
| | | | leader), |
| | | | 114 (w/out |
| | | | leader, no |
| | | | terminal |
| | | | lysine) |
| RON-f01 | 4C04 murine VL | 122 | 152 |
| | 4C04 murine VL CDR1 | | 141 |
| | 4C04 murine VL CDR2 | | 142 |
| | 4C04 murine VL CDR3 | | 143 |
| RON-f01 | 4C04 murine VH | 123 | 153 |
| | 4C04 murine VH variant | | 176 |
| | (terminal L−>S) | | |
| | 4C04 murine VH CDR1 | | 144 |
| | 4C04 murine VH CDR2 | | 145 |
| | 4C04 murine VH CDR3 | | 146 |
| RON-f01h2 | Humanized 4C04 murine VL | 124 | 154 |
| | using X59315 human | | |
| | germline framework | | |
| RON-f01h4 | Humanized 4C04 murine VH | 125 | 155 |
| | using Z12305 human | | |
| | germline framework | | |
| RON-f01h5 | Humanized 4C04 murine VH | 126 | 156 |
| | using Z14309 human | | |
| | germline framework | | |
| Ron-f02 | 4C04 murine VLVH scFV | 127 | 157 |
| RON-f07h24 | 4C04 Humanized VLVH | 128 | 158 |
| | scFV | | |
| RON-f08h25 | 4C04 Humanized VLVH | 129 | 159 |
| | scFV | | |
| RON-f02 | 4C04 murine SMIP: | 130 | 160 (w/ |
| | | | leader), |
| | | | 161 (w/out |
| | | | leader), |
| | | | 162 (w/out |
| | | | leader, no |
| | | | terminal |
| | | | lysine) |
| RON-f07h24 | Humanized 4C04 SMIP: | 131 | 163 (w/ |
| | f01h2 VL/f01h4 VH | | leader), |
| | | | 164 (w/out |
| | | | leader), |
| | | | 165 (w/out |
| | | | leader, no |
| | | | terminal |
| | | | lysine) |
| RON-f08h25 | Humanized 4C04 SMIP: | 132 | 166 (w/ |
| | f01h2 VL/f01H5 VH | | leader), |
| | | | 167 (w/out |
| | | | leader), |
| | | | 168 (w/out |
| | | | leader, no |
| | | | terminal |
| | | | lysine) |

The Interceptor pairs generated using the 4C04- and 11H09-derived RON binding domains are summarized in Table 4 below.

TABLE 4
Exemplary RON Interceptors

| | | Chain 1 ID and format (long | Chain 2 ID (short chain with no |
|---|---|---|---|
| Interceptor Characteristics | Interceptor ID | chain containing binding domain) | binding domain except where noted) |
| Chain 1-4C04 scFv BD | RON-f03 | VLVH—CH1—H—CH2—CH3-Ck | Ck-H—CH2—CH3—CH1 |
| Chain 2-Interceptor Pair 2 | | PN SEQ ID NO: 133 | PN SEQ ID NO: 11, 12 (w/and |
| | | AA SEQ ID NOS: 169 | w/out leader, respectively) |
| | | (w/leader), 170 (w/out leader) | AA SEQ ID NO: 34, 35 (w/and |
| | | | w/out leader, respectively) |
| Chain 1-4C04 scFv BD | RON-f04 | VLVH—H—CH2—CH3—CH1 | H—CH2—CH3-Ck(YAE) |
| Chain 2-Interceptor Pair 1C-1 | | PN SEQ ID NO: 134 | PN SEQ ID NO: 8 |
| | | AA SEQ ID NOS: 171 | AA SEQ ID NO: 26, 27 (w/and |
| | | (w/leader), 172 (w/out leader) | w/out leader, respectively) |
| Chain 1-4C04 scFv BD | RON-f05 | VLVH—CH1—H—CH2—CH3 | Ck(YAE)-H—CH2—CH3 |
| Chain 2-Interceptor Pair 1N-1 | | PN SEQ ID NO: 135 | PN SEQ ID NO: 9 |
| | | AA SEQ ID NOS: 173 | AA SEQ ID NO: 28, 29, 30 (w/ |
| | | (w/leader), 174 (w/out leader), | leader, w/out leader, and w/out |
| | | 175 (w/out leader/no terminal | leader w/out terminal Lys, |
| | | Lys) | respectively) |
| Chain 1-4C04 scFv BD | RON-f06 | VLVH—CH1—H—CH2—CH3 | Ck(EAE)-H—CH2—CH3 |
| Chain 2-Interceptor Pair 1N-2 | | PN SEQ ID NO: 135 | PN SEQ ID NO: 10 |
| | | AA SEQ ID NOS: 173 | AA SEQ ID NO: 31, 32, 33 (w/ |
| | | (w/leader), 174 (w/out leader), | leader, w/out leader, and w/out |
| | | 175 (w/out leader/no terminal | leader w/out terminal Lys, |
| | | Lys) | respectively) |
| Chain 1-11H09 scFv BD | RON-e03 | VLVH—CH1—H—CH2—CH3-Ck | Ck-H—CH2—CH1 |
| Chain 2- Interceptor Pair 2 | | PN SEQ ID NO: 57 | PN SEQ ID NO:11, 12 (w/and |
| | | AA SEQ ID NOS: 115 | w/out leader, respectively) |
| | | (w/leader), 116 (w/out leader) | AA SEQ ID NO: 34, 35 (w/and |
| | | | w/out leader, respectively) |
| Chain 1-11H09 scFv BD | RON-e04 | VLVH—H—CH2—CH3—CH1 | H—CH2—CH3-Ck(YAE) |
| Chain 2-Interceptor Pair 1C-1 | | PN SEQ ID NO: 58 | PN SEQ ID NO: 8 |
| | | AA SEQ ID NOS: 117 | AA SEQ ID NO:26, 27 (w/ and |
| | | (w/leader), 118 (w/out leader) | w/out leader, respectively) |
| Chain 1-11H09 scFv BD | RON-e05 | VLVH—CH1—H—CH2—CH3 | Ck(YAE)-H—CH2-CH3 |
| Chain 2-Interceptor Pair 1N-1 | | PN SEQ ID NO: 59 | PN SEQ ID NO: 9 |
| | | AA SEQ ID NOS: 119 | AA SEQ ID NO: 28, 29, 30 (w/ |
| | | (w/leader), 120 (w/out leader) | leader, w/out leader, and w/out |
| | | 121 (w/out leader/no terminal | leader w/out terminal Lys, |
| | | Lys) | respectively) |
| Chain 1-11H09 scFv BD | RON-e06 | VLVH—CH1—H—CH2—CH3 | Ck(EAE)-H—CH2—CH3 |
| Chain 2-Interceptor Pair 1N-2 | | PN SEQ ID NO: 59 | PN SEQ ID NO: 10 |
| | | AA SEQ ID NOS: 119 | AA SEQ ID NO: 31, 32, 33 (w/ |
| | | (w/leader), 120 (w/out leader) | leader, w/out leader, and w/out |
| | | 121 (w/out leader/no terminal | leader w/out terminal Lys, |
| | | Lys) | respectively) |
| Chain 1-hu4C04 scFv BD | RON-f10h24 | VLVH—H—CH2/CH3 null-CH1 | VLVH—H—CH2/CH3 null-CkYAE |
| Chain 2-huCris7 scFv BD* | | PN SEQ ID NO: 787 | PN SEQ ID NO: 807 |
| | | AA SEQ ID NO: 789 | AA SEQ ID NO: 808 |
| | | (sequences include leader) | |

*Anti-CD3 binding domain; see also published application WO2010/042904 for further description of the Cris7 monoclonal antibody and binding domains derived therefrom.

### Example 2

### RON-e01 and RON-f01 Murine Antibodies and Binding Molecules Derived Therefrom Specifically Bind Human RON and Cross-React with Macaca mulatta RON

The antibodies, SMIP, and Interceptor binding molecules generated as described in Example 1 were shown to bind human RON and to cross-react with Macaca mulatta (Mamu) RON.

NIH/3T3 cells transfected with human or macaque RON or empty vector, were dissociated with trypsin and stained at 1.6×10⁵ cells/sample on ice with hybridoma supernatants or purified antibodies diluted in Staining Buffer (2% FBS in Dulbecco's PBS). Unlabeled murine IgG (SouthernBiotech, Birmingham, Ala.) and the DX07 anti-RON β-chain antibody (Santa Cruz Biotechnology, Santa Cruz, Calif.) were employed as negative and positive controls respectively. Murine antibodies were detected with R-PE-conjugated goat anti-mouse IgG (SouthernBiotech). Samples were analyzed on a BD FACSCalibur flow cytometer fitted with an HTS using PlateManager and CellQuest Pro software (BD Biosciences, San Jose, Calif.). Data was plotted using FlowJo software (Tree Star, Ashland, Oreg.).

Macaca mulatta lung 4 MBr-5 cells (ATCC) were dissociated with Cell Dissociation Buffer Enzyme-Free PBS-based (Invitrogen) and stained at 1.1×10⁵ cells/sample with purified molecules diluted in Staining Buffer. SMIPs were detected with Alexa Fluor 488-conjugated goat anti-human IgG (Invitrogen), and dead cells were labeled with 20 μg/ml propidium iodide during the secondary antibody staining. Samples were analyzed on a BD FACSCalibur flow cytometer using CellQuest Pro software (BD Biosciences, San Jose, Calif.). Data (dead cells excluded) was plotted in FlowJo.

Human pancreatic adenocarcinoma BxPC-3 cells (ATCC) were dissociated with trypsin and human breast metastatic carcinoma MDA-MB-453 cells (ATCC) were harvested manually with a rubber cell scraper. Cells were stained at 3×10⁵ cells/sample on ice with purified molecules diluted in Staining Buffer. SMIPs and Interceptors were detected with Alexa Fluor 488-conjugated goat anti-human IgG (Invitrogen). Samples were analyzed on a BD FACSCalibur flow cytometer fitted with an HTS using PlateManager and CellQuest Pro software.

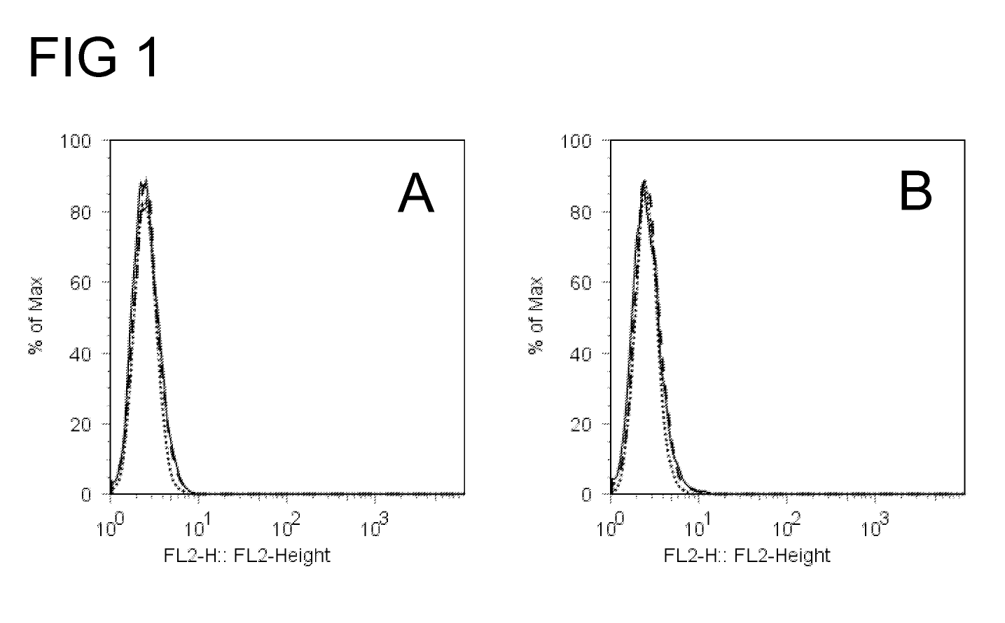

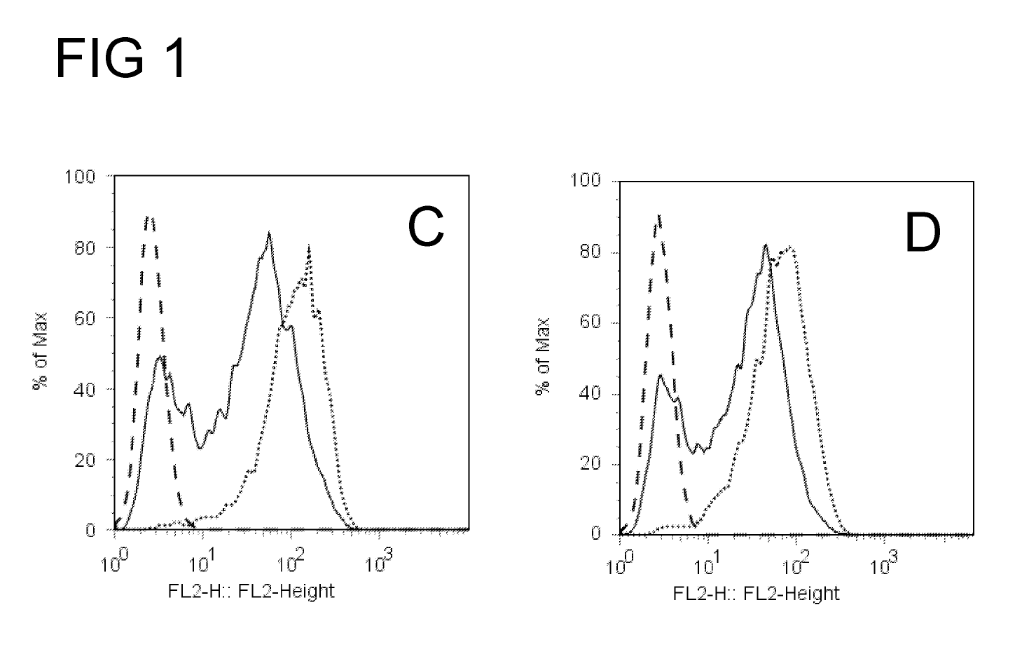

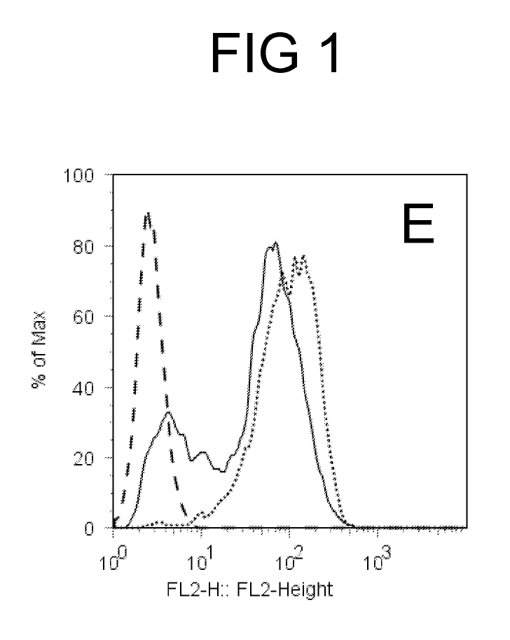

As shown in FIG. 1, RON-e01 and -f01 murine antibodies specifically bind human RON and cross-react with Macaca mulatta (Mamu) RON. NIH/3T3 cells transfected with empty vector (dashed), human RON (dotted) or Macaca mulatta RON (solid) were stained with secondary antibody alone (FIG. 1A), 1 mg/ml murine IgG (FIG. 1B), 1 mg/ml DX07 anti-RON antibody (FIG. 1C), RON-e01 anti-RON hybridoma supernatant (FIG. 1D) or RON-f01 anti-RON hybridoma supernatant (FIG. 1E).

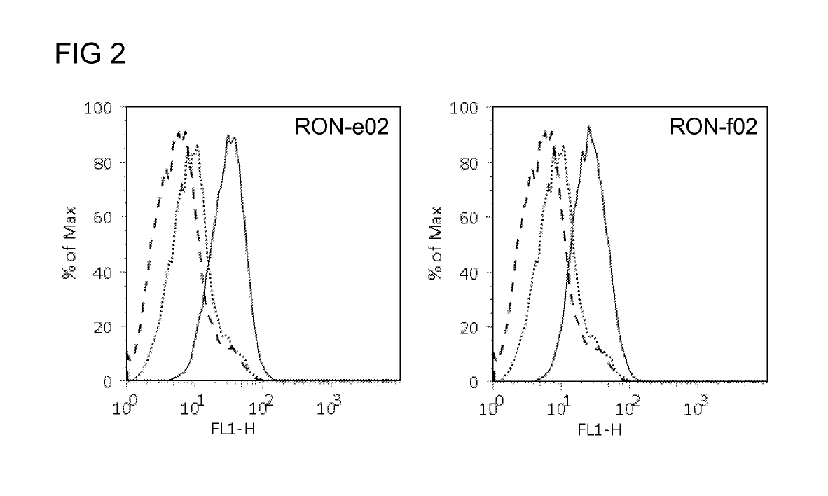

As demonstrated in FIG. 2, RON-e02 and -f02 SMIPs bind native Mamu RON on the surface of 4 MBr-5 cells. 4 MBr-5 cells were stained with secondary alone (dashed), the M0077 anti-CD79b SMIP (dotted), or anti-RON SMIP (solid).

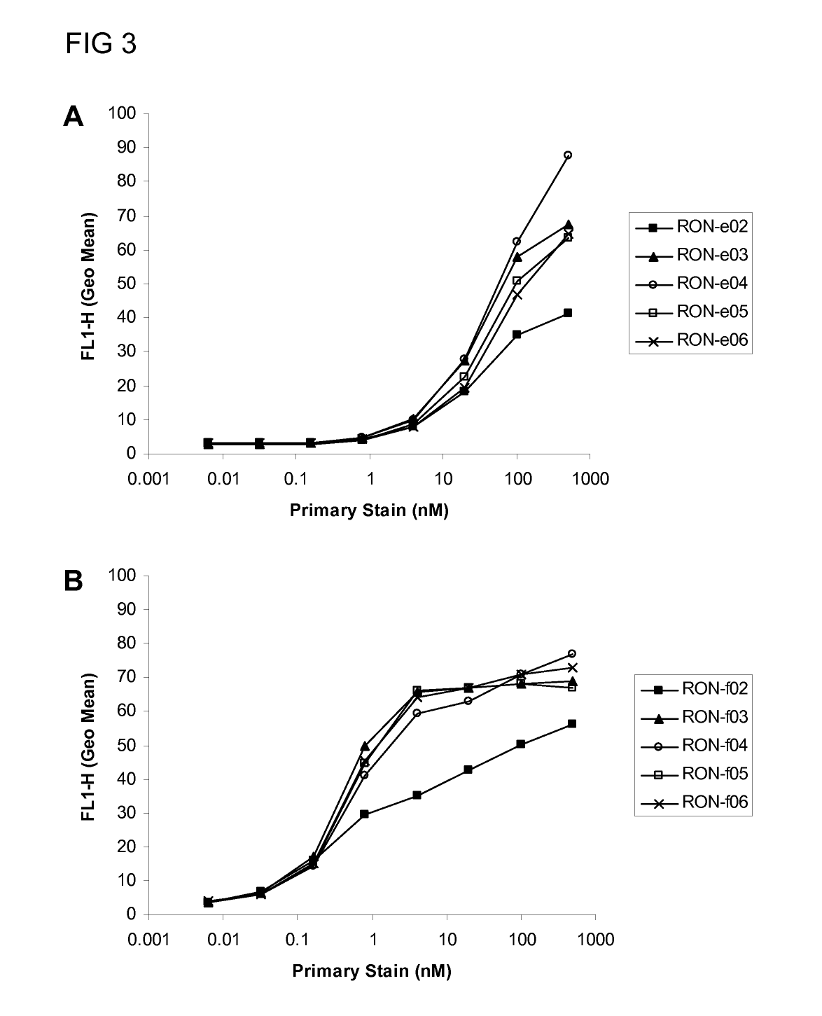

Furthermore, FIG. 3 shows that RON-e and RON-f SMIPs and Interceptor binding molecules bind native human RON on the surface of BxPC-3 cells. BxPC-3 cells were stained with various concentrations of RON-e (FIG. 3A) or RON-f (FIG. 3B) molecules. See Tables 3 and 4 for description of SMIPS and Interceptor constructs and associated SEQ ID NOs. RON Interceptors bind with a higher saturation level than their SMIP counterparts. This difference in saturation levels is likely to reflect a difference in RON receptor occupancy. While each Interceptor contains one binding domain and binds to a single RON molecule (a 1:1 binding ratio), each SMIP contains two binding domains and may occupy up to two RON molecules simultaneously (a 1:2 ratio).

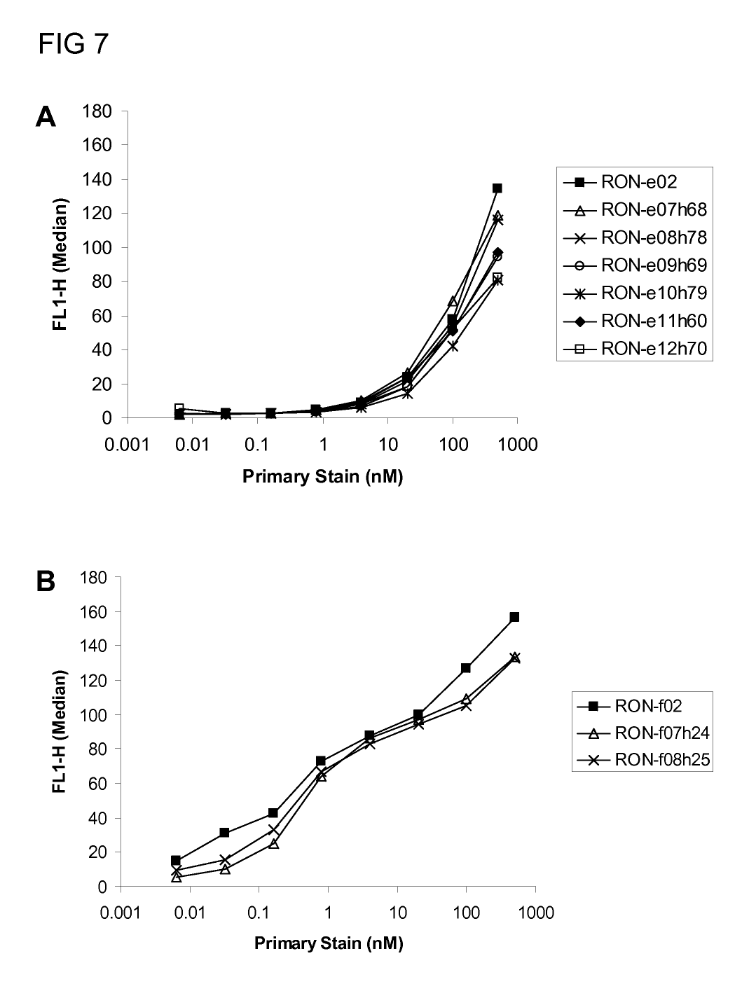

Additionally, RON-e (FIG. 7a) and RON-f (FIG. 7B) humanized SMIPs bind native human RON on the surface of MDA-MB-453 cells. Various concentrations of humanized RON-e SMIP constructs RON-e07h68, RON-e08h78, RON-e09h69, RON-e10h79, RON-e11h60, RON-e12h70 and RON-f SMIP SMIP constructs RON-f07h24 and RON-f08h25 were incubated with MDA-MB_{—}453 cells and compared with murine RON-e02 and RON-f02 controls, respectively. The humanized RON SMIPs have comparable binding activity as their murine counterparts.

These experiments demonstrate that the RON-e01 and RON-f01-based binding molecules specifically bind to human and macaque RON molecules in their native conformation.

### Example 3

### RON-e01 and RON-f01 Murine Antibodies Bind Different Epitopes Within the Extracellular Domain of RON

Anti-RON murine antibodies were tested for binding to the Sema-PSI domain of RON using ELISA.

To measure relative antibody concentration in hybridoma supernatant clones, 96-well EIA/RIA microplates (Corning Life Sciences, Lowell, Mass.) were coated with Goat F(ab′)₂ anti-mouse IgG (SouthernBiotech) and blocked with 10% FBS in DPBS prior to adding hybridoma supernatants diluted 1100 in serum diluent (DPBS/0.1% Tween 20/0.1% BSA). Murine antibodies captured by the coating antibody were detected with HRP-conjugated Goat anti-mouse IgM+IgG+IgA (SouthernBiotech), developed with TMB substrate (Thermo Fisher), and stopped with 1 N sulfuric acid. Plates were read at 450 nm on a VersaMax microplate reader (Molecular Devices, Sunnyvale, Calif.).

To determine binding of murine antibodies to the Sema-PSI domain of RON, 96-well EIA/RIA microplates were coated with 1 μg/ml recombinant RON Sema-PSI (R&D Systems #1947-MS, Minneapolis, Minn.). This protein includes the Sema and PSI domains of human RON (Glu 25-Leu 571; see SEQ ID NO:784) coupled to a carboxyl-terminal histidine tag and expressed in the NS0 mouse myeloma cell line. Plates were blocked with 10% FBS in DPBS prior to adding hybridoma supernatants diluted 15 in serum diluent. Murine antibodies bound to recombinant RON Sema-PSI were detected as described above.

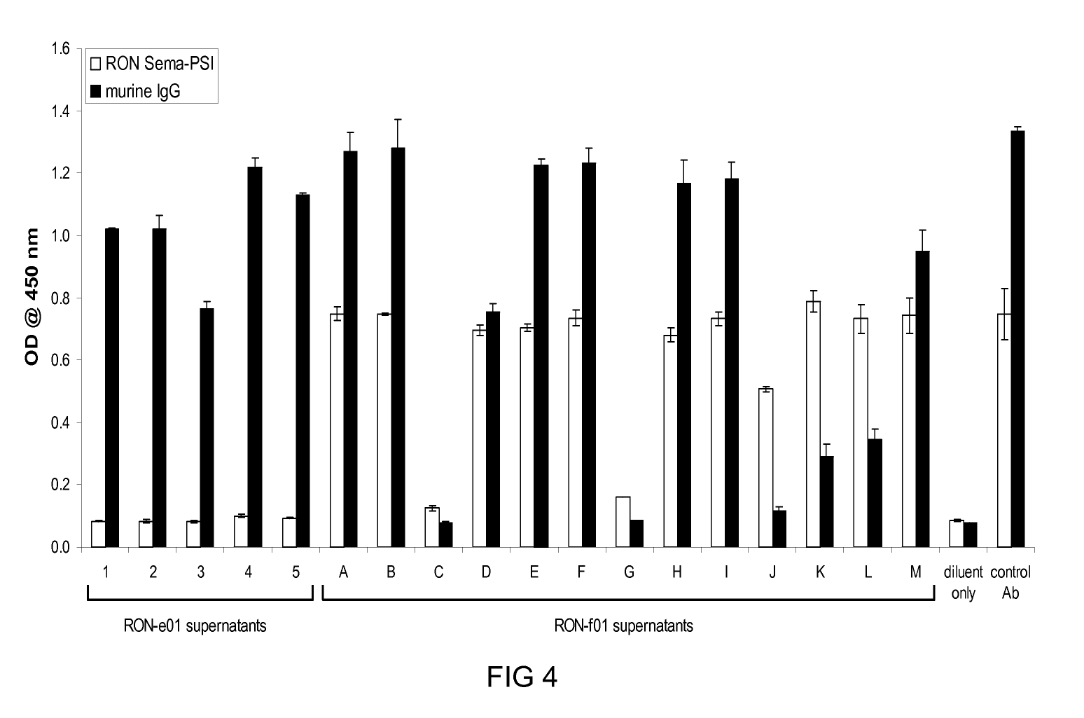

As shown in FIG. 4, RON-e01 antibody from hybridoma clone supernatants (1-5) containing measurable concentrations of IgG does not bind recombinant RON Sema-PSI protein, indicating that part or all of the epitope recognized by RON-e01 lies outside of the Sema and PSI domains. However, recombinant RON Sema-PSI protein binding is observed in all RON-f01 hybridoma clone supernatants (A-M) that contain measurable concentrations of IgG, suggesting that part or all of the epitope recognized by RON-f01 is contained within the RON Sema and PSI domains. “Diluent only” samples represent background binding in each assay when only serum diluent was run as the sample. As a positive control for IgG measurement and recombinant RON Sema-PSI binding, 250 ng/ml of an anti-human RON antibody (R&D Systems #MAB691, Minneapolis, Minn.) was tested in both ELISAs.

### Example 4

### RON-e and RON-f Binding Molecules do not Compete with Each Other for Cell Surface Binding

BxPC-3 cells dissociated with trypsin were stained on ice with molecules diluted in Staining Buffer (2% FBS in DPBS). 3×10⁵ cells were incubated on ice for 1 hour with 500 nM of competitor molecule, washed, and stained with 100 nM primary murine antibody or SMIP prior to detection with an Alexa Fluor 488-conjugated anti-mouse or anti-human IgG secondary respectively (RON-e01: murine antibody; RON-f02: anti-RON SMIP; DX07: anti-RON n-chain antibody (Santa Cruz Biotechnology, Santa Cruz, Calif.). Samples were analyzed on a BD FACSCalibur flow cytometer using CellQuest Pro software.

As shown in FIG. 5, RON-e and RON-f molecules do not compete with each other for cell surface binding, confirming the results of Example 3 showing that RON-e and RON-f molecules bind RON at different epitopes. DX07 and RON-f molecules interfere with each other's cell surface binding, suggesting that they may bind similar regions of RON or prevent binding through steric hindrance.

### Example 5

### RON-e and RON-f Binding Molecules can Inhibit MSP-Induced Phosphorylation of RON, Akt and MAPK

Western blot analysis of phosphoproteins was used to determine whether RON binding molecules could inhibit MSP-induced phosphorylation of RON, AKT and MAPK.

MDA-MB-453 cells were plated at 2.5×10⁶ cells/well in 6-well plates in DMEM+10% FBS overnight. The following day, media was aspirated and replaced with 10 or 200 nM blocking treatments prepared in serum-free RPMI for 1 hour at 37° C. Blocking treatments were aspirated and cells were stimulated with MSP (R&D Systems, Minneapolis, Minn.) for 30 min at 37° C. Both no ligand and 3 nM MSP treatments were prepared in serum-free RPMI media with 100 μM Na₃VO₄. Cells were washed once with ice-cold TBS (50 mM Tris-HCl pH 8, 150 mM NaCl) and lysed on ice in 150 μL RIPA Lysis Buffer (Thermo Fisher) supplemented with HALT™ Protease Inhibitor Cocktail, HALT™ Phosphatase Inhibitor Cocktail, 5 mM EDTA, 1 mM Na₃VO₄ and 0.9 mM phenylmethylsulfonyl fluoride. Lysates were clarified by centrifugation at 4° C. and processed for denaturing electrophoresis. 17.5 μl RIPA lysate was loaded per lane and separated on Tris-Glycine gels of 6% (RON) or 4-20% (Akt and MAPK). Gels were blotted onto nitrocellulose membranes. All anti-RON antibodies were obtained from Santa Cruz Biotechnology (Santa Cruz Biotechnology, Santa Cruz, Calif.), and all anti-Akt and MAPK antibodies were from Cell Signaling Technology (Danvers, Mass.). Secondary antibodies were purchased from LI-COR Biosciences (Lincoln, Nebr.).

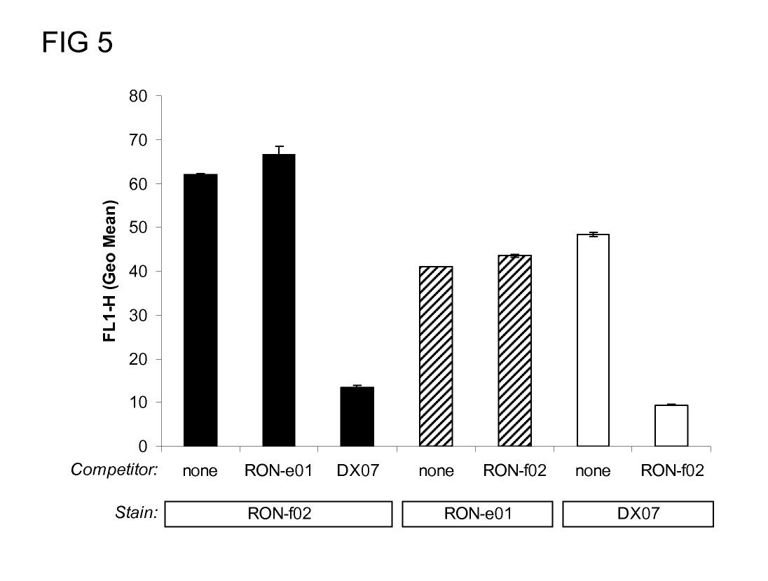

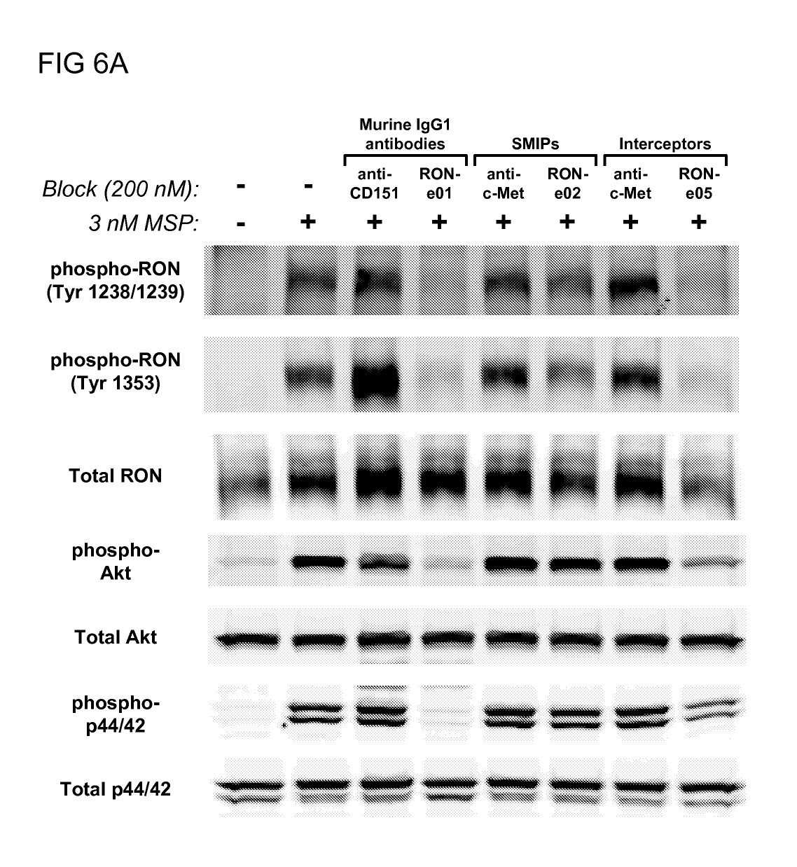

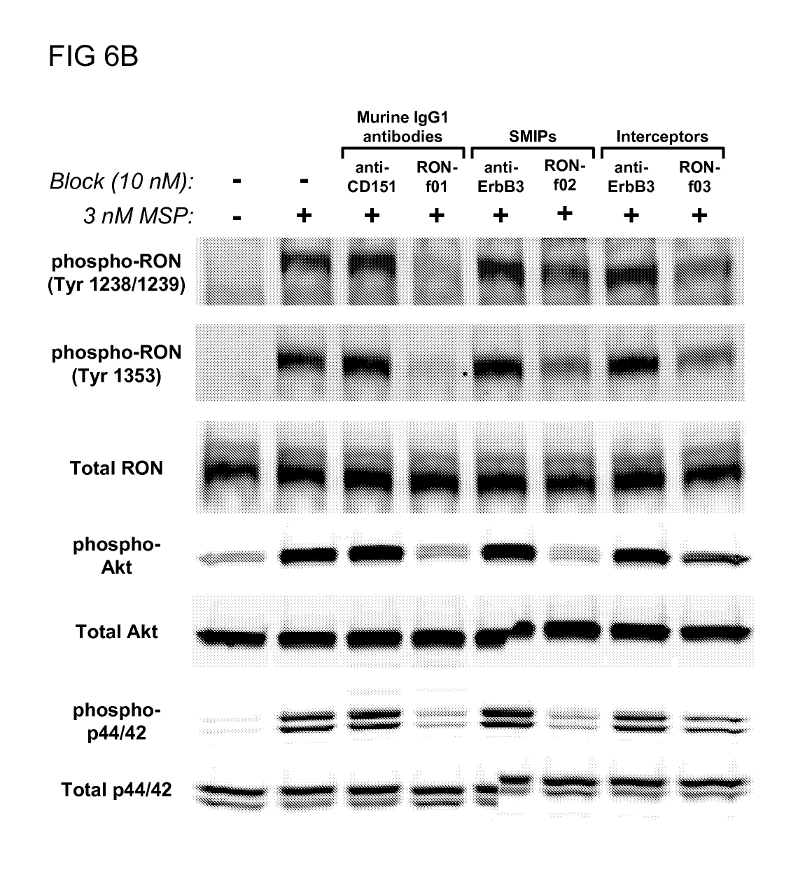

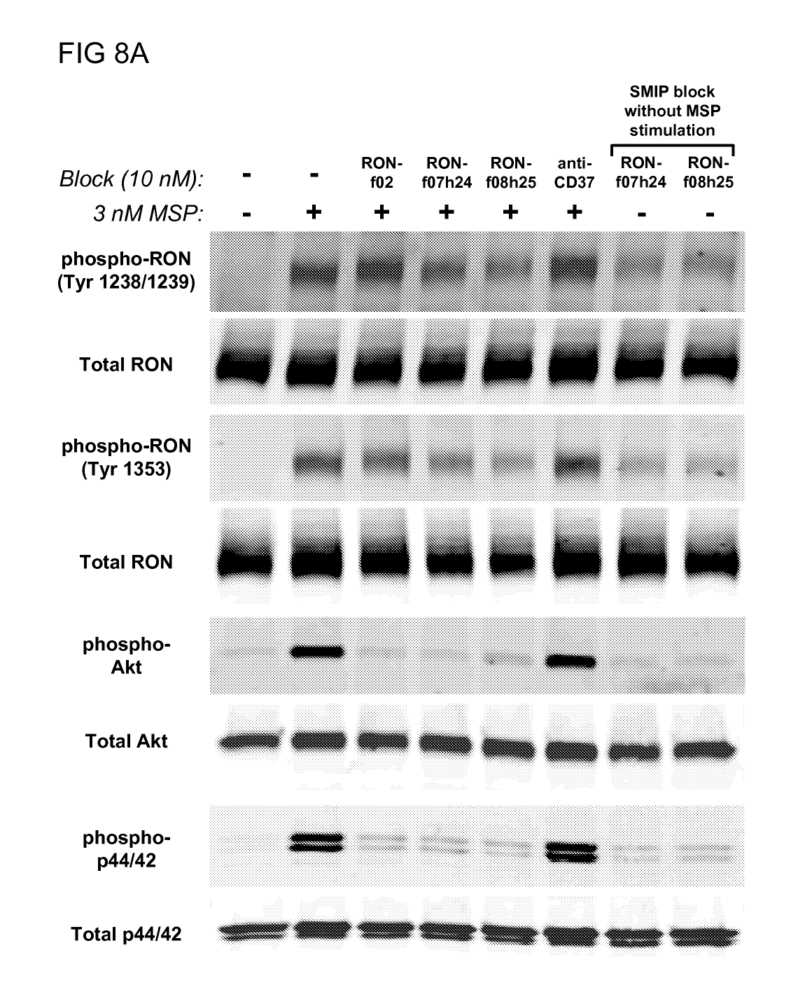

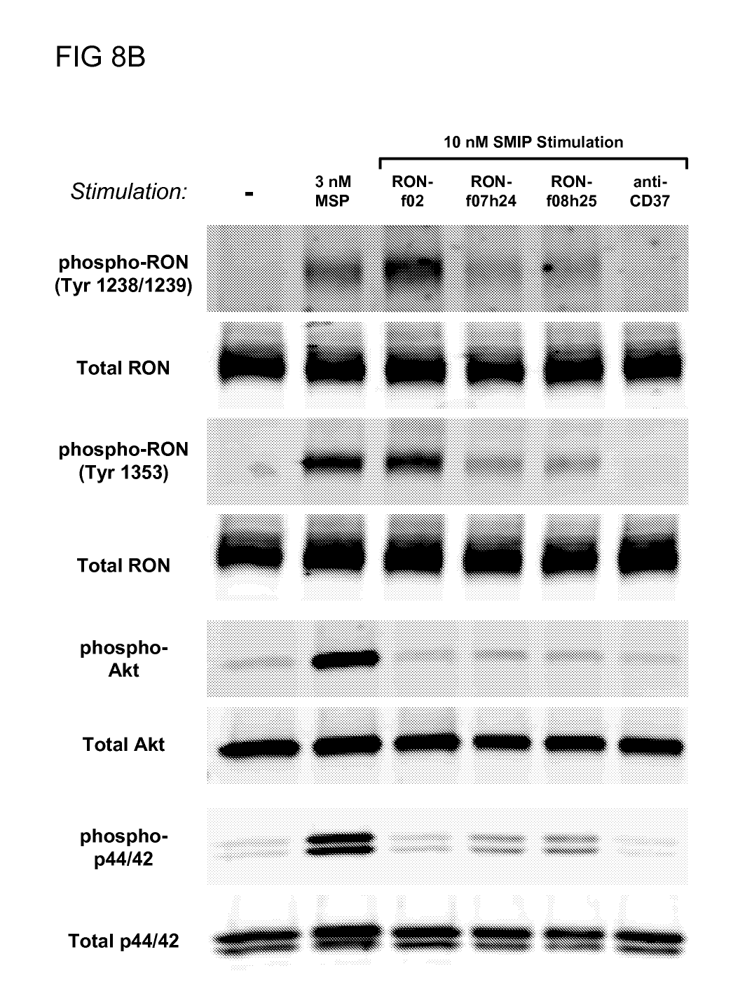

Tyrosine-phosphorylated RON was detected on duplicate blots using anti-phosphoRON antibodies against phospho-tyrosines 1238/1239 or 1353 and IRDye 800CW donkey anti-rabbit or anti-goat secondary antibodies, respectively. The anti-phospho-tyrosine 1238/1239 and/or 1353 blots were re-probed for total RON using the RON β C-20 antibody and an IRDye 680 (FIG. 6) or 800CW (FIG. 8) donkey anti-rabbit secondary. Phospho-Akt (Ser473) and phospho-p44/42 MAPK (Thr202/Tyr204) were detected on the same blot with IRDye 680 donkey anti-rabbit or IRDye 800CW donkey anti-mouse secondary antibodies, respectively. Either a duplicate blot (FIG. 6) or the anti-phospho-Akt/MAPK blots (FIG. 8) were probed for total Akt and MAPK using pan Akt 40D4 and p44/42 MAPK antibodies detected with IRDye 680 donkey anti-mouse or IRDye 800CW donkey anti-rabbit secondary antibodies, Fh respectively. Blots were analyzed using the ODYSSEY® Infrared Imaging System (LI-COR, Lincoln, Nebr.).

As shown in FIG. 6A, RON-e01 antibody and RON-e05 YAE Interceptor can inhibit MSP-induced phosphorylation of RON, Akt and MAPK while RON-e02 SMIP exhibits unremarkable blocking activity. Additionally, RON-f01 antibody, RON-f02 SMIP and the RON-f03 Interceptor can inhibit MSP-induced phosphorylation of RON, Akt and MAPK (FIG. 6B).

As shown in FIG. 8A, RON-f humanized SMIPs (RON-f07h24 and RON-f08h25) can inhibit MSP-induced phosphorylation of RON, Akt, and MAPK in MDA-MB-453 cells. RON-f humanized SMIPs cause minimal phosphorylation of RON, but not of Akt or MAPK when applied during the blocking step and followed by mock stimulation. FIG. 8B shows that humanization of the RON-f02 murine SMIP reduces receptor phosphorylation in response to SMIP application during the stimulation step. RON-f02 murine SMIP stimulates RON phosphorylation but not downstream Akt or MAPK phosphorylation. The humanized SMIPs (RON-f07h24 and RON-f08h25) caused reduced RON phosphorylation compared to the murine SMIP RON-f02. Interestingly, the high level of downstream effector protein phosphorylation observed in response to MSP-induced RON activation is not observed following SMIP-induced phosphorylation of the RON receptor.

Therefore, the RON binding molecules described herein may be used for inhibiting MSP-induced signaling pathways and thus are useful in a variety of therapeutic settings including for the therapy of various cancers, such as, but not limited to, pancreatic cancer.

### Example 6

### Binding Kinetics of RON-e and RON-f Binding Molecules

Binding kinetics of the RON-e and RON-f binding molecules were determined using Biacore analysis.

The RON Sema-PSI-AFH protein was produced in CHOK1SV cells (Lonza, Allendale, N.J.) stably transfected with a construct encompassing the Sema-PSI region of RON (a.a. 25-568) fused to a c-terminal tag including avidin, 3×FLAG®, and 6× histidine tags. The soluble RON protein included a thrombin cleavage site (LVPRG; SEQ ID NO:177) substituted for the native cleavage site (KRRRR; SEQ ID NO:178) at amino acids 305-309. The protein was purified from supernatant using anti-FLAG® M2 Affinity Agarose Gel (Sigma-Aldrich, St. Louis, Mo.), eluted with 3×FLAG® Peptide (Sigma-Aldrich) and further purified by Size Exclusion Chromatography (SEC).

The binding kinetics of RON-f murine antibody, SMIP and Interceptor to soluble RON Sema-PSI-AFH were determined using a Biacore T100 (GE Healthcare, Piscataway, N.J.). Anti-RON murine antibody was captured using immobilized anti-mouse Fc polyclonal antibody while the SMIP and Interceptor were captured by anti-human Fc monoclonal antibody. The capture antibodies, both from GE Healthcare, were covalently conjugated to a carboxylmethyl dextran surface (CM4) via amines using N-ethyl-N′-(3-dimethylaminopropyl)-carbodiimide hydrochloride and N-hydroxysuccinimide. The unoccupied sites of the activated surface were blocked by ethanolamine. The capturing antibodies showed no discernible dissociation from the captured anti-RON molecules during the course of the assay. During each cycle, a single concentration of soluble RON Sema-PSI-AFH was injected and then allowed to dissociate. At the end of each cycle, the surface was regenerated gently using 3M MgCl₂ which dissociates protein bound to the capture antibodies. Signal associated with binding to the reference cell was used to subtract for bulk refractive changes and blank (buffer-only) injections were used to correct for drift and system noise.

Kinetic parameters and affinities were determined using BIAevaluation software. The kₐ (M⁻¹s⁻¹) and k_{d} (s⁻¹) rates of the interaction were used to calculate the affinity constant, K_{D} (M), of the antibody/receptor interaction. The K_{D} is defined as the ratio of the k_{d} and kₐ constants (k_{d}/kₐ). The RON-f01 murine antibody was tested in a single experiment while the RON-f02 SMIP and RON-f03 Interceptor molecules were each tested in three independent experiments. Rate and affinity constants from a representative experiment are shown in Table 5. RON-f molecules were captured on a sensor chip with immobilized anti-Fc while soluble RON Sema-PSI-AFH protein was flowed over the surface at varying concentrations.

TABLE 5
Rate and affinity constants for RON-f molecules
determined by Biacore analysis.

| Sample | kₐ (M⁻¹ · s⁻¹) | k_{d} (s⁻¹) | K_{D} (pM) |
|---|---|---|---|
| RON-f01 | 6.32 × 10⁵ | 9.49 × 10⁻⁵ | 150 |
| RON-f02 | 8.66 × 10⁵ | 1.00 × 10⁻⁴ | 115 |
| RON-f03 | 4.34 × 10⁵ | 6.78 × 10⁻⁵ | 156 |

### Example 7

### RON-e and RON-f Binding Molecules Prevent Complete MSP-Induced Wound Healing of BxPC-3 Cells

The ability of RON binding molecules to prevent MSP-induced wound healing was tested using an in vitro functional assay.

BxPC-3 cells were plated at 5×10⁵ cells/well into collagen-coated 24-well plates (BD Biosciences, San Jose, Calif.) in 1 ml of RPMI+10% FBS and incubated for 18 hours at 37° C. The next day, media was aspirated from the cells and replaced with 1 ml of sterile DPBS. The cell monolayer was scratched vertically down the center of each well with a 1-ml pipet tip. After making the scratch, the DPBS and any dislodged cells were carefully aspirated from the well. Each well received 500 μl of serum-free RPMI or blocking reagent diluted to 100 μM in serum-free RPMI. Cells were incubated for 1 hour at 37° C. During the blocking step, the plates were imaged for the 0-hour time point using an IN Cell Analyzer 1000 (GE Healthcare, Piscataway, N.J.) with the bright field setting and a 4× objective. MSP ligand (R&D Systems, Minneapolis, Minn.) was diluted to 5 μg/ml in serum-free RPMI. Following the blocking incubation, 10 μl of serum-free RPMI (no ligand control) or diluted MSP was added to each well for a final concentration of 100 ng/ml MSP/well. The plates were incubated for 18 hours at 37° C. and imaged again on the IN Cell Analyzer using settings identical to the 0-hour time point. Wounds were scored for complete healing (as observed with MSP stimulation in the absence of blocking treatment) or incomplete healing (as observed in the absence of MSP stimulation). Each treatment was performed in duplicate. The results are summarized in Tables 6 and 7.

TABLE 6
RON-e and RON-f proteins prevent MSP-induced wound healing of BxPC-3 cells.

| | Controls and Irrelevant Proteins | | RON-e Proteins | | RON-f Proteins | |
|---|---|---|---|---|---|---|
| | Blocking | Wound | Blocking | Wound | Blocking | Wound |
| MSP | Treatment | Healing | Treatment | Healing | Treatment | Healing |
| − | — | Incomplete | — | — | — | — |
| + | — | Complete | — | — | — | — |
| + | anti-CD28 | Complete | RON-e01 | Incomplete | RON-f01 | Incomplete |
| | Antibody | | Antibody | | Antibody | |
| + | anti-CD28 | Complete | RON-e02 | Incomplete | RON-f02 | Incomplete |
| | SMIP | | SMIP | | SMIP | |
| + | anti-CD28 | Complete | RON-e03 | Incomplete | RON-f03 | Incomplete |
| | Interceptor | | Interceptor | | Interceptor | |

TABLE 7
Humanized RON-e and RON-f SMIPs prevent MSP-induced wound healing of BxPC-3 cells.

| Controls and Irrelevant SMIP | | | RON-e SMIPs | | | RON-f SMIPs | | |
|---|---|---|---|---|---|---|---|---|
| | Blocking | Wound | | Blocking | Wound | | Blocking | Wound |
| MSP | Treatment | Healing | MSP | Treatment | Healing | MSP | Treatment | Healing |
| − | — | Incomplete | + | RON-e02 | Incomplete | + | RON-f02 | Incomplete |
| + | — | Complete | + | RON-e07h68 | Incomplete | + | RON-f07h24 | Incomplete |
| + | anti-CD37 | Complete | + | RON-e08h78 | Incomplete | + | RON-f08h25 | Incomplete |
| | SMIP | | | | | | | |
| | | | + | RON-e09h69 | Incomplete | | | |
| | | | + | RON-e10h79 | Incomplete | | | |
| | | | + | RON-e11h60 | Incomplete | | | |
| | | | + | RON-e12h70 | Incomplete | | | |

As summarized in Tables 6 and 7, RON binding domain molecules including the anti-RON-e01 anti-RON-f01 antibodies, the RON-e02 and RON-f02 SMIPs, and the RON-e03 and RON-f03 Interceptors molecules, and the humanized RON-e and RON-f SMIPs all blocked MSP-induced wound healing BxPC-3 cells.

### Example 8

### Bispecific Humanized RON-f Binding Domain/Anti-CD3 Binding Domain Molecules Specifically Direct Cytotoxic T Cell Killing of Target Cells Expressing the RON Antigen

In this example, a directed T cell cytotoxicity assay was used to demonstrate that bispecific molecules having a RON binding domain and an anti-CD3 binding domain could direct cytotoxic T cell-mediated killing of target cells expressing RON. Two different anti-RON binding domain molecule formats were used. In particular, a RON binding SCORPION molecule and a RON binding Interceptor molecule were constructed. The f10h24 RON binding Interceptor molecule is described in Table 4 and the polynucleotide and amino acid sequences for this construct are set forth in SEQ ID NOs:787 and 789, respectively. The single chain anti-CD3 Interceptor pair polypeptide comprises from its amino to carboxyl terminus: CRIS7 (anti-CD3 monoclonal antibody) scFv, human IgG1 SCC-P hinge, human IgG1 CH2(ADCC/CDC null), human IgG1 CH3, and human Ck(YAE). The nucleotide and amino acid sequences of this construct are set forth in SEQ ID NOS:807 and 808, respectively. The SCORPION construct is comprised of the humanized 4C04 ScFv and a humanized Cris7 ScFv and contains an Fc domain having mutations that abrogate ADCC and CDC activity. The nucleotide and amino acid sequences of the SCORPION construct are set forth in SEQ ID NOs:786 and 788, respectively.

MDA-MB-453 (ATCC) and Daudi (ATCC) target cells were loaded with 0.05 mCi of Chromium-51 per million cells. The target cells were washed and re-suspended to a concentration of 2×10⁵ cells/mL in Assay Media [RPMI 1640, 10% FBS, 1 mM sodium pyruvate, 1×MEM non-essential amino acids (Invitrogen), 55 μM 2-mercaptoethanol]. T cells of healthy donors were isolated from peripheral blood mononuclear cells using the Pan T Cell Isolation Kit II (Miltenyi Biotec, Auburn, Calif.). Unstimulated T cells were washed and re-suspended in Assay Media at 1×10⁶ cells/mL. In 96-well U bottom plates, 50 μL target cells (10,000 cells/well) were combined with 50 μL 4× treatment (Assay Media alone, NP-40 detergent, or bispecific protein) and incubated at room temperature for 15 min. 100 μL Assay Media or T cells (100,000 cells/well) were added to wells as appropriate and the plates were incubated at 37° C., 5% CO₂ for 4 hours. Following the incubation, the cells were pelleted gently and 25 μL of cell-free supernatant was transferred to scintillator coated LUMAPLATE™-96 plates (PerkinElmer, Waltham, Mass.). The scintillation plates were dried overnight and counts per minute (cpm) for each sample were recorded on a TopCount NXT (PerkinElmer). Spontaneous release was measured in wells containing target cells, T cells, and no treatment. Total lysis was measured in wells containing target cells and 0.2% NP-40 detergent. Data was plotted as ° A) total lysis, determined according to the following equation:

$$\% Total Lysis = \frac{( {cpm}^{sample} - {cpm}^{spontaneous release} )}{( {cpm}^{total lysis} - {cpm}^{spontaneous release} )}$$

As shown in FIGS. 9A and 9B, both target cell lines were killed by T cells only when incubated together with T cells and a bispecific protein that binds an antigen expressed by the target cell. When the bispecific protein does not bind the target cell (i.e. an anti-RON×anti-CD3 bispecific with Daudi cells or anti-CD19 with MDA-MB-453 cells), no target cell cytotoxicity was observed. Thus, bispecific proteins pairing a humanized RON-f binding domain with an anti-CD3 binding domain specifically direct cytotoxic T cell killing of target cells expressing the RON antigen. These experiments demonstrate that RON binding molecules paired with an anti-CD3 binding domain molecule as described herein may be used in a therapeutic setting to recruit cytotoxic T cells to kill target cells expressing RON.

### Example 9

### Polypeptide Heterodimers with Anti-RON and Anti-c-MET Binding Domains

A bivalent polypeptide heterodimer with anti-RON binding domains (ORN151) and two bispecific polypeptide heterodimers comprising anti-RON and anti-cMet binding domains (ORN152 and ORN153) were made.

Bivalent polypeptide heterodimer ORN151 comprises single chain polypeptides ORN145 (4C04 CH2 CH3 CH1) and ORN148 (11H09CH2 CH3 Ck(YAE)). Single chain polypeptide ORN145 comprises from its amino to carboxyl terminus: 4C04 (anti-RON) scFv, human IgG1 SCC-P hinge, human IgG1 CH2, human IgG1 CH3 and human IgG1 CH1. The nucleotide and amino acid sequences of ORN145 are set forth in SEQ ID NOS:810 and 811, respectively. Single chain polypeptide ORN148 comprises from its amino to carboxyl terminus: 11H09 (anti-RON) scFv, human IgG1 SCC-P hinge, human CH2, human CH3, and human Ck(YAE). The nucleotide and amino acid sequences of ORN148 are set forth in SEQ ID NOS:812 and 813, respectively.

Bispecific (c-Met, RON) polypeptide heterodimer ORN152 comprises single chain polypeptides ORN116 (MET021 CH2 CH3 CH1) and ORN146 (4C04 CH2 CH3 Ck(YAE)). Single chain polypeptide ORN116 comprises from its amino to carboxyl terminus: MET021 (anti-c-Met) scFv, human IgG1 SCC-P hinge, human IgG1 CH2, human IgG1 CH3 and human IgG1 CH1. The nucleotide and amino acid sequences of ORN116 are set forth in SEQ ID NOS:814 and 815, respectively. Single chain polypeptide ORN146 comprises from its amino to carboxyl terminus: 4C04 (anti-RON) scFv, human IgG1 SCC-P hinge, human CH2, human CH3, and human Ck(YAE). The nucleotide and amino acid sequences of ORN146 are set forth in SEQ ID NOS:816 and 817, respectively.

Bispecific (c-Met, RON) polypeptide heterodimer ORN153 comprises single chain polypeptides ORN116 (MET021 CH2 CH3 CH1) and ORN148 (11H09CH2 CH3 Ck(YAE)).

Polypeptide heterodimers ORN151, ORN152 and ORN153 were expressed according to the method below. The following expression levels were obtained: 1.9 μg protein/mL of culture for ORN151, 3.1 μg/mL for ORN152, and 4.9 μg/mL for ORN153.

### Expression

The day before transfection, HEK293 cells were suspended at a cell concentration of 0.5×10⁶ cells/ml in Freestyle 293 expression medium (Gibco). For a large transfection, 250 ml of cells were used, but for a small transfection, 60 ml of cells were used. On the transfection day, 320 ul of 293fectin reagent (Invitrogen) was mixed with 8 ml of media. At the same time, 250 ug of DNA for each of the two chains were also mixed with 8 ml of media and incubated for 5 minutes. After 15 minutes of incubation, the DNA-293fectin mixture was added to the 250 ml of 293 cells and returned to the shaker at 37° C. and shaken at a speed of 120 RPM. For the smaller transfection using 60 ml of cells, a fourth of the DNA, 293fectin and media were used.

### Example 10

### Cell Binding of Bispecific Anti-RON/Anti-CD3 Polypeptide Heterodimers

The polypeptide heterodimer S0268, with anti-RON and anti-CD3 binding domains, was constructed. S0268 comprises single chain polypeptides ORN145 (4C04 CH2 CH3 CH1) and TSC019 (G19-4 CH2 CH3 Ck(YAE)). Single chain polypeptide TSC019 comprises from its amino to carboxyl terminus: G19-4 (anti-CD3) scFv, human IgG1 SCC-P hinge, human CH2, human CH3, and human Ck(YAE). The nucleotide and amino acid sequences of TSC019 are set forth in SEQ ID NOS:818 and 819, respectively. Nucleotide and amino acid sequences of the ORN145 single chain polypeptides are set forth in SEQ ID NOS:810 and 811, respectively.

To compare the effectiveness of bispecific polypeptide heterodimer molecules at targeting a tumor cell antigen and T-cells, the on-cell binding characteristics of S0268 with a different bispecific scaffold (SCORPION™ protein) containing the same binding domains, S0266, were compared. The nucleotide and amino acid sequences of SCORPION protein S0266 are set forth in SEQ ID NOS:820 and 821, respectively. Transient transfection in human 293 cells produced 6.9 μg protein/mL of culture for S0266; 2.3 μg/mL of culture for S0268; 3.0 μg/mL of culture for TSC020; and 3.2 μg/mL of culture for TSC021.

MDA-MB-453 (RON+) breast carcinoma cells were obtained from ATCC (Manassas, Va.), and cultured according to the provided protocol. T-cells were isolated from donor PBMCs using a Pan T-cell Isolation Kit II from Miltenyi Biotec (Bergisch Gladbach, Germany). Non T-cells were separated from PBMCs by being indirectly magnetically labeled with biotin-conjugated monoclonal antibodies and anti-biotin magnetic microbeads. These cells were then depleted by retaining them in a column surrounded by a magnetic field. The T-cells were not retained in the column and were collected in the flow through.

Binding was assessed by incubating 5×10⁵ T cells or target (MDA-MB-453) cells for 30 minutes at 4° C. with serially diluted bispecific molecules S0266 (αRON×αCD3 SCORPION™ protein) or S0268 (αRON×αCD3 polypeptide heterodimer) (for MDA-MB-453 cells and isolated T cells), in concentrations from 100 nM to 0.1 nM. The cells were washed three times and then incubated with goat anti-human IgG-FITC (1:200 dilution) for another 30 minutes at 4° C. The cells were then washed again three times, fixed in 1% paraformaldehyde and read on a FACS-Calibur instrument.

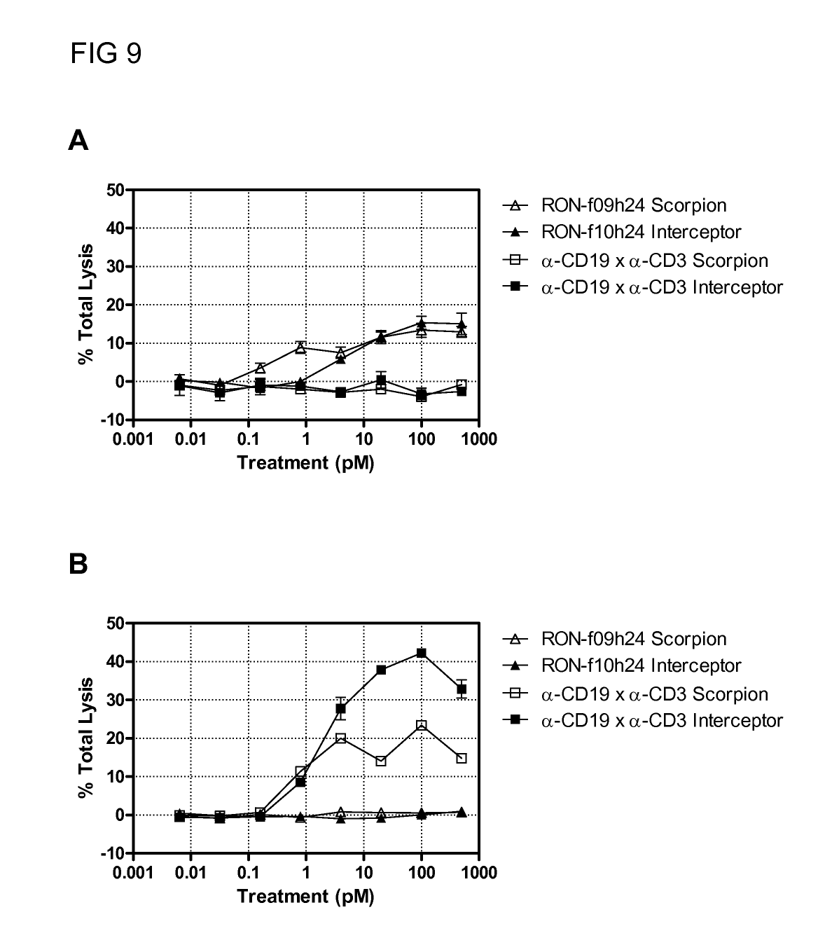

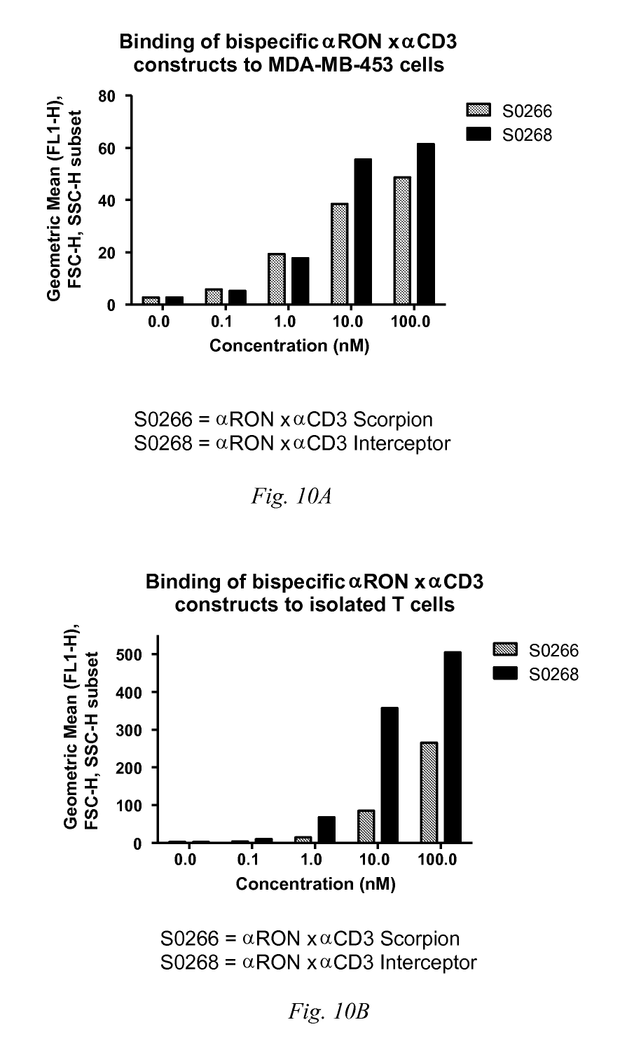

Analysis of the FSC high, SSC high subset in FlowJo v7.5 (Tree Star, Inc, Ashland, Oreg.) showed dose-dependent binding of bispecific molecules S0266 and S0268 to both MDA-MB-453 and isolated T-cells (FIGS. 10A and 10B). Unexpectedly, the S0268 polypeptide heterodimer bound with similar affinity to the comparable SCORPION™ molecule (S0266) on both MDA-MB-453 cells and T-cells, although it lacked the potential for any avidity. Higher saturation on both target cell types was also observed with the polypeptide heterodimer, which would be the case if the polypeptide heterodimer was binding at a higher stoichometry (1:1 binding of polypeptide heterodimer to surface antigen) than the equivalent SCORPION™ (potential 1:2 binding of the bivalent Scorpion to surface antigens).

### Example 11

### Redirected T-Cell Cytotoxicity by Polypeptide Heterodimers

To compare the effectiveness of different bispecific polypeptide heterodimer molecules at inducing target-dependent T-cell cytotoxicity, four different bispecific molecules were compared in a chromium (⁵¹Cr) release assay. Three different bispecific molecules (TSC054, TSC078, TSC079) with a common anti-CD19 binding domain (HD37) and three different anti-CD3 binding domains (G19-4 for TSC054, OKT3 for TSC078, HuM291 for TSC079) were tested alongside a fourth bispecific molecule (S0268, see Example 10) with an anti-RON binding domain (4C04) and an anti-CD3 binding domain (G19-4). Bivalent polypeptide heterodimer TSC054 comprises single chain polypeptides TSC049 (HD37 CH2(ADCC/CDC null) CH3 CH1) and TSC053 (G19-4 CH2(ADCC/CDC null) CH3 Ck(YAE)). Single chain polypeptide TSC049 comprises from its amino to carboxyl terminus: HD37 (anti-CD19) scFv, human IgG1 SCC-P hinge, human IgG1 CH2(ADCC/CDC null) (i.e., human IgG1 CH2 with L234A, L235A, G237A, E318A, K320A, and K322A substitutions), human IgG1 CH3, and human IgG1 CH1. The nucleotide and amino acid sequences of TSC049 are set forth in SEQ ID NOS:822 and 823, respectively. Single chain polypeptide TSC053 comprises from its amino to carboxyl terminus: G19-4 (anti-CD3) scFv, human IgG1 SCC-P hinge, human IgG1 CH2(ADCC/CDC null) (i.e., human IgG1 CH2 with L234A, L235A, G237A, E318A, K320A, and K322A substitutions), human IgG1 CH3, and human Ck(YAE). The nucleotide and amino acid sequences of TSC053 are set forth in SEQ ID NOS:824 and 825, respectively.

Bivalent polypeptide heterodimer TSC078 comprises single chain polypeptides TSC049 (HD37 CH2(ADCC/CDC null) CH3 CH1) and TSC076 (OKT3 CH2(ADCC/CDC null) CH3 Ck(YAE)). Single chain polypeptide TSC076 comprises from its amino to carboxyl terminus: OKT3 (anti-CD3) scFv, human IgG1 SCC-P hinge, human IgG1 CH2(ADCC/CDC null), human IgG1 CH3, and human Ck(YAE). The nucleotide and amino acid sequences of TSC076 are set forth in SEQ ID NOS:826 and 827, respectively.

Bivalent polypeptide heterodimer TSC079 comprises single chain polypeptides TSC049 (HD37 CH2(ADCC/CDC null) CH3 CH1) and TSC077 (Nuvion CH2(ADCC/CDC null) CH3 Ck(YAE)). Single chain polypeptide TSC077 comprises from its amino to carboxyl terminus: Nuvion (anti-CD3) scFv, human IgG1 SCC-P hinge, human IgG1 CH2(ADCC/CDC null), human IgG1 CH3, and human Ck(YAE). The nucleotide and amino acid sequences of TSC077 are set forth in SEQ ID NOS:828 and 829, respectively.

Transient transfection in human 293 cells produced about 2.33 μg/mL protein for TSC054, about 0.67 μg/mL protein for TSC078, and about 3.5 μg/mL protein for TSC079.

Daudi Burkitt's lymphoma cells (CD19+, RON−) and BxPC-3 cells (CD19−, RON+) were obtained from ATCC (Manassas, Va.) and cultured according to the provided protocol. Peripheral blood mononuclear cells (PBMC) were isolated from human blood using standard ficoll gradients. The isolated cells were washed in saline buffer. T cells were additionally isolated using a Pan T-cell Isolation Kit II from Miltenyi Biotec (Bergisch Gladbach, Germany) using the manufacturer's protocol (see also Example 5 for more information).

Cytotoxicity was assessed by a ⁵¹Cr release assay. Approximately 5×10⁶ Daudi or BxPC-3 cells were treated with 0.3 mCi of ⁵¹Cr and incubated for 75 minutes at 37° C. After 75 minutes, cells were washed 3 times with media (RPMI+10% FBS) and resuspended in 11.5 mL of media. From this suspension, 50 μL was dispensed per well into 96 well U-bottom plates (approximately 20,000 cells/well). Concentrations of bispecific molecules ranging from 10 nM to 0.1 μM were added to the target (Daudi, BxPC-3) cells, bringing the total volume to 100 μL/well. Target cells were incubated at room temperature for 15 minutes. Then 100 μL of isolated T-cells (approximately 200,000) were added to bring the T-cell to target cell ratio to 10:1. 50 μL of 0.8% NP-40 was added to a control well containing target cells, left for 15 minutes, then 100 μL of media was added to provide a total lysis control.

Plates were incubated for 4 hours, spun at 1500 rpm for 3 minutes, and 25 μL of supernatant was transferred from each well to the corresponding well of a 96-well Luma sample plate. Sample plates were allowed to air dry in a chemical safety hood for 18 hours, and then radioactivity was read on a Topcount scintillation counter using a standard protocol.

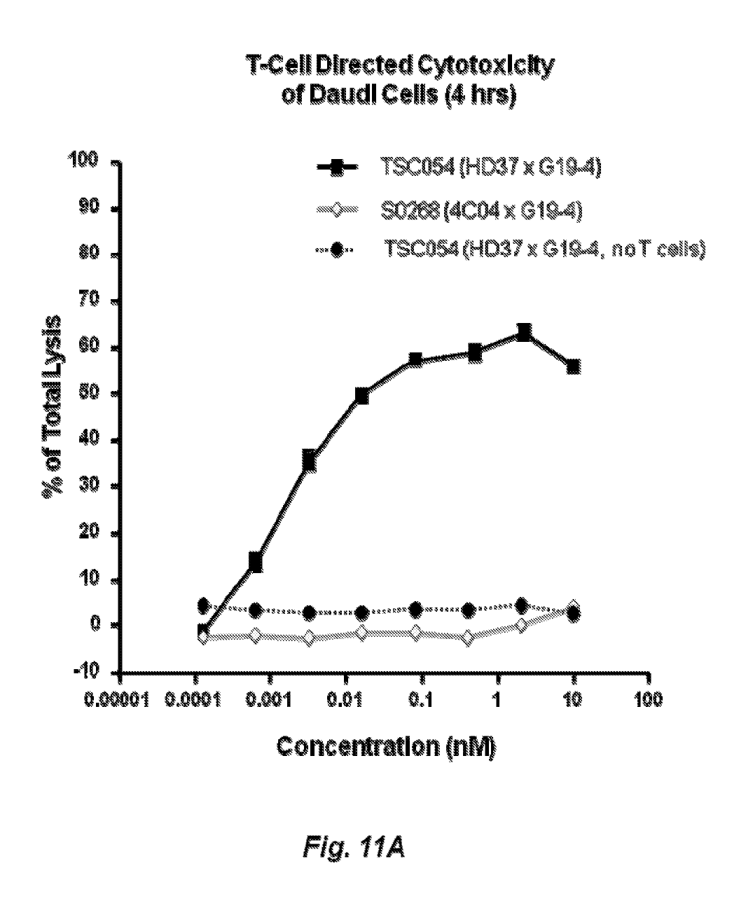

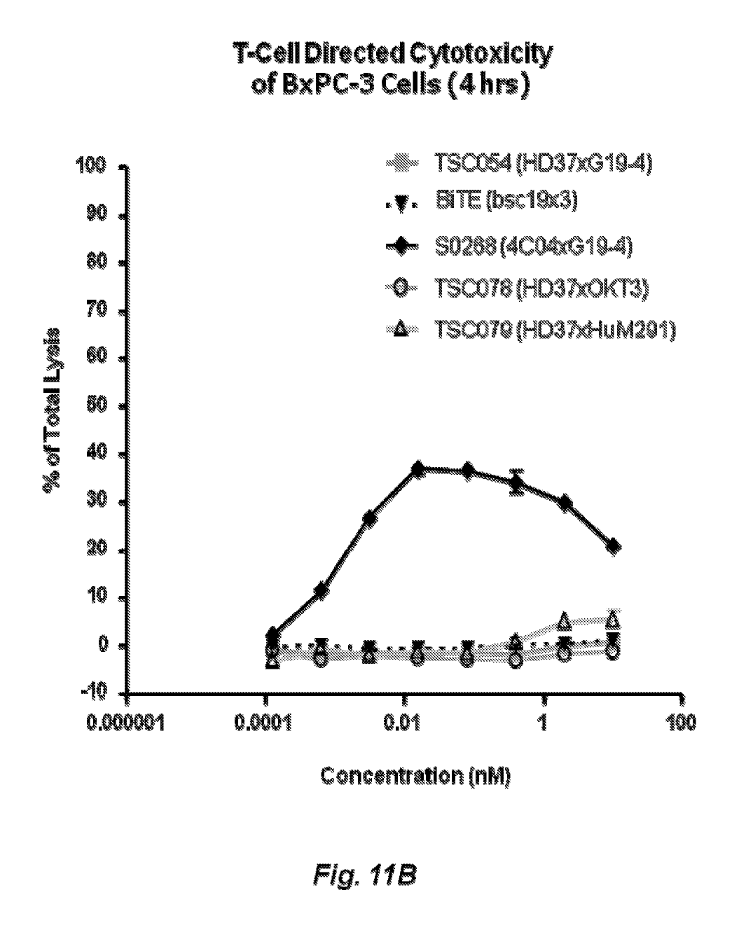

Analysis of cytotoxicity data showed a lack of off-target cytotoxicity on the Daudi (RON−) cells from the anti-RON directed bispecific molecule S0268 (FIG. 11A). Similarly, there was a lack of direct cytotoxicity observed from treating Daudi cells with TSC054 in the absence of T-cells (FIG. 11A). However, strong T-cell directed cytotoxicity was observed with the Daudi cells in the presence of T-cells and an anti-CD19 directed bispecific molecule (TSC054), reaching maximal lysis at a concentration between 10 and 100 μM (FIG. 11A). Similarly, using a second T-cell donor (FIG. 11B), no off-target cytotoxicity of the BxPC-3 (CD19−) cells was observed from the CD19-directed bispecifics TSC054, TSC078, or TSC079, or the CD19-directed BiTE bsc19x3. The anti-RON directed S0268 bispecific molecule induced cytotoxicity in BxPC-3 (RON+) cells, reaching a maximum between 10 and 100 μM (FIG. 11B).

### Example 12

### Bispecific Anti-RON/Anti-CD19 Polypeptide Heterodimer

A bivalent anti-RON/anti-CD19 polypeptide heterodimer, TSC099, was constructed. TSC099 comprises single chain polypeptides TSC049 (anti-CD19) (HD37 CH2(ADCC/CDC null) CH3 CH1) and TSC097 (4C04 CH2(ADCC/CDC null) CH3 Ck(YAE)). Single chain polypeptide TSC097 comprises from its amino to carboxyl terminus: 4C04 (anti-RON) scFv, human IgG1 SCC-P hinge, human IgG1 CH2(ADCC/CDC null), human IgG1 CH3, and human Ck(YAE). The nucleotide and amino acid sequences of TSC097 are set forth in SEQ ID NOS:830 and 831, respectively. Single chain polypeptide TSC049 comprises from its amino to carboxyl terminus: HD37 (anti-CD19) scFv, human IgG1 SCC-P hinge, human IgG1 CH2(ADCC/CDC null) (i.e., human IgG1 CH2 with L234A, L235A, G237A, E318A, K320A, and K322A substitutions), human IgG1 CH3, and human IgG1 CH1. The nucleotide and amino acid sequences of TSC049 are set forth in SEQ ID NOS:822 and 823, respectively.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-35. (canceled)

36. A multi-specific fusion protein comprising (i) a RON binding domain, (ii) a CD3 binding domain and (iii) an immunoglobulin constant region or sub-region thereof between the RON and CD3 binding domains.

37. The multi-specific fusion protein of claim 36, wherein the immunoglobulin constant region or sub-region thereof comprises an immunoglobulin Fc region or an immunoglobulin CH2CH3 domain.

38. The multi-specific fusion protein of claim 37, wherein the immunoglobulin CH2CH3 domain is from an IgG1, IgG2, IgG3, IgG4, IgA1, IgA2 or IgD.

39. The multi-specific fusion protein of claim 36, wherein the multi-specific fusion protein does not comprise an immunoglobulin CH1 domain.

40. The multi-specific fusion protein of claim 37, wherein the immunoglobulin Fc region or CH2CH3 domain comprises
(i) an amino acid substitution at the asparagine of position 297 and one amino acid substitution at position 234, 235, 236 or 237;
(ii) an amino acid substitution at the asparagine of position 297 and amino acid substitutions at two of positions 234-237;
(iii) an amino acid substitution at the asparagine of position 297 and amino acid substitutions at three of positions 234-237;
(iv) an amino acid substitution at the asparagine of position 297, amino acid substitutions at positions 234, 235 and 237, and an amino acid deletion at position 236;
(v) amino acid substitutions at three of positions 234-237 and amino acid substitutions at positions 318, 320 and 322; or
(vi) amino acid substitutions at three of positions 234-237, an amino acid deletion at position 236, and amino acid substitutions at positions 318, 320 and 322.

41. The multi-specific fusion protein of claim 36, wherein the multi-specific fusion protein comprises a hinge domain.

42. The multi-specific fusion protein of claim 41, wherein the multi-specific fusion protein comprises from N-terminus to C-terminus (a) a RON binding domain, (b) a hinge domain, (c) an immunoglobulin constant region or sub-region thereof, and (d) a CD3 binding domain or from N-terminus to C-terminus (a) a CD3 binding domain, (b) a hinge domain, (c) an immunoglobulin constant region or sub-region thereof, and (d) a RON binding domain.

43. The multi-specific fusion protein of claim 36, wherein the multi-specific fusion protein comprises a dimerization domain.

44. The multi-specific fusion protein of claim 36, wherein the RON binding domain comprises:
(a) a VL domain comprising
i. a CDR1 amino acid sequence of SEQ ID NO:67, a CDR2 amino acid sequence of SEQ ID NO:68, and a CDR3 amino acid sequence of SEQ ID NO:69; or
ii. a CDR1 amino acid sequence of SEQ ID NO:141, a CDR2 amino acid sequence of SEQ ID NO:142, and a CDR3 amino acid sequence of SEQ ID NO:143; or
(b) a VH domain comprising
i. a CDR1 amino acid sequence of SEQ ID NO:70, a CDR2 amino acid sequence of SEQ ID NO:71, and a CDR3 amino acid sequence of SEQ ID NO:72; or
ii. a CDR1 amino acid sequence of SEQ ID NO:144, a CDR2 amino acid sequence of SEQ ID NO:145, and a CDR3 amino acid sequence of SEQ ID NO:146; or
(c) a VL of (a) and a VH of (b).

45. The multi-specific fusion protein of claim 44, wherein the VL and VH domains are humanized.

46. The multi-specific fusion protein of claim 44, wherein the VL domain comprises an amino acid sequence of any one of SEQ ID NOS:80 and 152, and the VH domain comprises an amino acid sequence of any one of SEQ ID NOS:81, 153 and 176.

47. The multi-specific fusion protein of claim 44, wherein VL domain comprises an amino acid sequence of any one of SEQ ID NOS:82, 83 and 154, and the VH domain comprises an amino acid sequence of any one of SEQ ID NOS:84-86, 155 and 156.

48. The multi-specific fusion protein of claim 36, wherein the RON binding domain is an antibody or an antigen-binding fragment of an antibody.

49. The multi-specific fusion protein of claim 48, wherein the antibody or antigen-binding fragment of the antibody is non-human, chimeric, humanized or human.

50. The multi-specific fusion protein of claim 48, wherein the antibody or antigen-binding fragment of the antibody comprises a VL domain that is at least about 90% identical to any one of the amino acid sequences of SEQ ID NOS:80, 82, 83, 152 and 154 and comprises a VH domain that is at least about 90% identical to any one of the amino acid sequences of SEQ ID NOS:81, 84-86, 153, 155, 156 and 176.

51. The multi-specific fusion protein of claim 36, wherein the RON binding domain is selected from the group consisting of a Fab fragment, an F(ab′)2 fragment, an scFv, a dAb, and a fragment.

52. The multi-specific fusion protein of claim 36, wherein the immunoglobulin constant region or sub-region thereof is disposed between a first linker peptide and a second linker peptide.

53. The multi-specific fusion protein of claim 52, wherein the first and second linker peptides are independently selected from the linkers provided in SEQ ID NOS:610-777.

54. The multi-specific fusion protein of claim 52, wherein the first linker peptide comprises an immunoglobulin hinge region and the second linker peptide comprises a type II C lectin stalk region.

55. The multi-specific fusion protein of claim 36 comprising the following structure:
N-BD1-X-L2-BD2-C wherein:
—X— is -L1-CH2CH3-, wherein L1 is an immunoglobulin IgG1 hinge having an amino acid sequence comprising any one of SEQ ID NOs:349-366 and 420-475 and wherein CH2CH3- is a human IgG1 CH2CH3 region or a variant thereof lacking one or more effector functions;
L2 is a linker peptide having an amino acid sequence comprising any one of SEQ ID NOS:610-777; and either
BD1 is the RON binding domain and BD2 is the CD3 binding domain or BD1 is the CD3 binding domain and BD2 is the RON binding domain.

56. The multi-specific fusion protein of claim 36, wherein the fusion protein comprises SEQ ID NO:788 or 821.

57. A composition comprising the multi-specific fusion protein of claim 36 and a pharmaceutically acceptable carrier, diluent, or excipient.

58. A polynucleotide encoding the multi-specific fusion protein of claim 36.

59. An expression vector comprising the polynucleotide according to claim 58 operably linked to an expression control sequence.

60. A host cell comprising the expression vector according to claim 59.

61. A method for treating cancer or an inflammatory disorder comprising administering to a subject in need thereof a therapeutically effective amount of the composition of claim 57.

62. The method of claim 61, wherein the cancer is selected from the group consisting of pancreatic cancer, lung cancer, colon cancer and breast cancer.
